(19) United States
(12) Patent Application Publication
(10) Pub. No.: US 2022/0396836 A1
(43) Pub. Date: Dec. 15, 2022

(54) METHODS AND SYSTEMS FOR MENSTRUALOME ANALYSIS

(71) Applicant: NextGen Jane, Inc., Oakland, CA (US)

(72) Inventors: Stephen GIRE, Oakland, CA (US); Ridhi TARIYAL, Oakland, CA (US); Trevor RUGGIERO, Oakland, CA (US); Adrienne CLARK, Oakland, CA (US); Corbin MUNN, Oakland, CA (US); Jacob FREAKE, Oakland, CA (US)

(21) Appl. No.: 17/772,829

(22) PCT Filed: Oct. 30, 2020
(86) PCT No.: PCT/US2020/058406

§ 371 Date: Apr. 28, 2022

Related U.S. Application Data
(60) Provisional application No. 63/061,709, filed on Aug. 5, 2020.
(60) Provisional application No. 62/930,465, filed on Nov. 4, 2019.
(60) Provisional application No. 62/929,579, filed on Nov. 1, 2019.

(51) Int. Cl. C12Q 1/6883; A61B 5/15; G01N 33/76

(52) U.S. Cl. CPC C12Q 1/6883; A61B 5/150755; G01N 33/76; A61B 10/0045

(57) ABSTRACT

Samples, systems for collecting samples, and methods of preserving samples from menstrual fluid are provided.

### CROSS-REFERENCE

This application claims the benefit of U.S. Patent Application No. 62/929,579, filed Nov. 1, 2019, U.S. Patent Application No. 62/930,465, filed Nov. 4, 2019, and U.S. Patent Application No. 63/061,709, filed Aug. 5, 2020, which are hereby incorporated by reference in their entirety.

### BACKGROUND OF THE DISCLOSURE

Chronic pelvic pain (CPP), dysmenorrhea, and infertility are symptoms that drive women to seek medical care for diseases related to an aberrant menstrual cycle, for example undiagnosed endometriosis. For example, the prevalence of endometriosis is up to 70% among women presenting with CPP, and 30-50% among women presenting at IVF clinics for infertility. The presentation of these symptoms along with the type of endometriosis disease often determines treatment options. Treatment options include surgery and pain management through hormone therapy and/or GnRH analogs. However, reimbursement coverage for GnRH analogs vary greatly and may be gated by a surgical confirmation of disease, making a surgical diagnosis of endometriosis a necessary step in receiving proper care. This significantly drives costs not only to the health system but to individuals suffering from the disease, and it contributes to the overall lag in time-to-diagnosis. Even with surgical intervention, 50% of patients have recurrence; underlining the fact that endometriosis shows periodic states of activation, regardless of surgical or therapeutic intervention.

It takes, on average, ten years from the onset of symptoms of endometriosis to diagnosis, which allows adhesions and scar tissue to form in the reproductive system, both hampering function and often causing severe pain. In addition, the combination of painful periods and a lack of clear diagnosis can cause psychological distress and depression in affected women.

### SUMMARY OF THE DISCLOSURE

In some embodiments, disclosed herein are methods for preparation of a menstrualome fingerprint. In some embodiments, the method comprises: (a) obtaining a first sample and a second sample from a subject, wherein the first sample and the second sample comprise cervicovaginal or menstrual fluid collected onto a first and second absorbent sample collector; (b) eluting the first sample and the second sample separately from the first and second sample collector into an aqueous buffer; (c) separating a biological material from each of the first sample and the second sample; and (d) constructing a sample menstrualome fingerprint, wherein the sample menstrualome fingerprint comprises the differential of the level and/or presence of a plurality of menstrualome biomarkers in the biological material from the first sample and the second sample. In some embodiments, the biological material comprises one or more biological materials selected from the group consisting of a RNA, a DNA, a methylated nucleic acid, a miRNA, a protein, a protein-nucleic acid complex, a microorganism, and a mammalian cell type. In some embodiments, constructing the sample menstrualome fingerprint in (d) comprises assaying the extracted biological material from the first sample and the second sample to identify a plurality of biomarkers. In some embodiments, the plurality of menstrualome biomarkers comprise biomarkers that display differential presence or level in cervicovaginal or menstrual fluid between two or more health states. In some embodiments, the plurality of menstrualome biomarkers comprise biomarkers that display differential presence or level in cervicovaginal or menstrual fluid as compared to peripheral blood, cervicovaginal tissue, or a longitudinal menstrual sample. In some embodiments, the method further comprises (e) comparing the sample menstrualome fingerprint to a reference menstrualome fingerprint. In some embodiments, the reference menstrualome fingerprint comprises a threshold level or presence of the plurality of menstrualome biomarkers that are associated with a health state. In some embodiments, the first sample and second sample comprise biological material collected at a different time points from the subject. In some embodiments, the time points are separated by a time period between about 15 minutes and about 30 days, about 60 days, or about 90 days. In some embodiments, the time points comprise different days within a menstrual cycle of the subject. In some embodiments, the time points are within a single menstrual cycle. In some embodiments, the time points comprise days in separate menstrual cycles. In some embodiments, the time points are during one or more days of menstruation of the subject. In some embodiments, one time point is during menstruation of the subject and one time point is not during menstruation of the subject. In some embodiments, the sample collector is an intravaginal sample collector. In some embodiments, the sample collector preserves a biological material in an intact state. In some embodiments, the sample collector is capable of absorbing at least 3 ml of fluid. In some embodiments, the sample collector is placed into a buffer subsequent to collecting the sample. In some embodiments, the biological material is DNA and plurality of menstrualome biomarkers comprises methylation status of a plurality of loci. In some embodiments, the biological material is RNA and plurality of menstrualome biomarkers comprises expression level of a plurality of genes. In some embodiments, the biological material is RNA and a plurality of menstrualome biomarkers comprises the presence and/or level of a plurality of miRNAs. In some embodiments, the biological material is cells and plurality of menstrualome biomarkers measures the presence and/or amount of one or more cell types. In some embodiments, the biological material is DNA and plurality of menstrualome biomarkers measures the presence and/or level of one or more microorganisms. In some embodiments, the biological material is DNA and plurality of menstrualome biomarkers measures the diversity of microorganisms. In some embodiments, the two or more health states comprise before and after a medical treatment. In some embodiments, the health state comprises a health state before surgery. In some embodiments, the reference state comprises a health state after surgery. In some embodiments, the health state comprises a menstrual disorder. In some embodiments, the health state comprises endometriosis. In some embodiments, the health state comprises a healthy patient. In some embodiments, the health reference menstrualome fingerprint comprises a principle component analysis, a t-Distributed Stochastic Neighbor Embedding, a heat map, a diversity index, or a combination thereof.

In another aspect, disclosed herein are methods for preparation of a menstrualome fingerprint. In some embodiments, the method comprises: (a) obtaining a first sample and a second sample from a subject, wherein the first sample and the second sample comprise cervicovaginal or menstrual fluid collected onto a first and second absorbent sample collector; (b) eluting the first sample and the second sample separately from the first and second sample collector into an aqueous buffer; (c) separating a biological material from each of the first sample and the second sample; and (d) constructing a sample menstrualome fingerprint, wherein the sample menstrualome fingerprint comprises the differential of the level and/or presence of a plurality of menstrualome biomarkers in the biological material from the first sample and/or the second sample as compared to a reference menstrualome fingerprint. In some embodiments, the biological material comprises one or more biological materials selected from the group consisting of a RNA, a DNA, a methylated nucleic acid, a miRNA, a protein, a protein-nucleic acid complex, a microorganism, and a mammalian cell type. In some embodiments, constructing the sample menstrualome fingerprint in (d) comprises assaying the extracted biological material from the first sample and the second sample to identify a plurality of biomarkers. In some embodiments, the plurality of menstrualome biomarkers comprise biomarkers that display differential presence or level in cervicovaginal or menstrual fluid between two or more health states. In some embodiments, the plurality of menstrualome biomarkers comprise biomarkers that display differential presence or level in cervicovaginal or menstrual fluid as compared to peripheral blood, cervicovaginal tissue, or a longitudinal menstrual sample. In some embodiments, the reference menstrualome fingerprint comprises a threshold level or presence of the plurality of menstrualome biomarkers that are associated with a health state. In some embodiments, the first sample and second sample comprise biological material collected at a different time points from the subject. In some embodiments, the time points are separated by a time period between about 15 minutes and about 30 days, about 60 days, or about 90 days. In some embodiments, the time points comprise different days within a menstrual cycle of the subject. In some embodiments, the time points are within a single menstrual cycle. In some embodiments, the time points comprise days in separate menstrual cycles. In some embodiments, the time points are during one or more days of menstruation of the subject. In some embodiments, one time point is during menstruation of the subject and one time point is not during menstruation of the subject. In some embodiments, the sample collector is an intravaginal sample collector. In some embodiments, the sample collector preserves a biological material in an intact state. In some embodiments, the sample collector is capable of absorbing at least 3 ml of fluid. In some embodiments, the sample collector is placed into a buffer subsequent to collecting the sample. In some embodiments, the biological material is DNA and plurality of menstrualome biomarkers comprises methylation status of a plurality of loci. In some embodiments, the biological material is RNA and plurality of menstrualome biomarkers comprises expression level of a plurality of genes. In some embodiments, the biological material is RNA and plurality of menstrualome biomarkers comprises the presence and/or level of a plurality of miRNAs. In some embodiments, the biological material is cells and plurality of menstrualome biomarkers measures the presence and/or amount of one or more cell types. In some embodiments, the biological material is DNA and plurality of menstrualome biomarkers measures the presence and/or level of one or more microorganisms. In some embodiments, the biological material is DNA and plurality of menstrualome biomarkers measures the diversity of microorganisms. In some embodiments, the two or more health states comprise before and after a medical treatment. In some embodiments, the health state comprises a health state before surgery. In some embodiments, the reference state comprises a health state after surgery. In some embodiments, the health state comprises a menstrual disorder. In some embodiments, the health state comprises endometriosis. In some embodiments, the health state comprises a healthy patient. In some embodiments, the health reference menstrualome fingerprint comprises a principle component analysis, a t-Distributed Stochastic Neighbor Embedding, a heat map, a diversity index, or a combination thereof.

In another aspect, disclosed herein are methods for preparation of a menstrualome fingerprint. In some embodiments, the method comprises: (a) obtaining a first sample from a subject, wherein the first sample comprise cervicovaginal or menstrual fluid collected onto an absorbent sample collector; (b) eluting the first sample from the sample collector into an aqueous buffer; (c) separating a biological material from the first sample; (d) constructing a sample menstrualome fingerprint, wherein the sample menstrualome fingerprint comprises the level and/or presence of a plurality of menstrualome biomarkers in the biological material from the first sample; and (e) comparing the sample menstrualome fingerprint to a reference fingerprint. In some embodiments, the reference fingerprint comprises the level and/or presence of a plurality of menstrualome biomarkers in a reference group of subjects. In some embodiments, the reference fingerprint comprises the level and/or presence of a plurality of menstrualome biomarkers in the subject at a prior time point. In some embodiments, reference menstrualome fingerprint comprises a threshold level or presence of the plurality of menstrualome biomarkers that are associated with a health state. In some embodiments, the reference menstrualome fingerprint comprises a threshold level or presence of the plurality of menstrualome biomarkers that are associated with a health state. In some embodiments, the plurality of menstrualome biomarkers comprise biomarkers that display differential presence or level in cervicovaginal or menstrual fluid between two or more health states. In some embodiments, the two or more health states comprise before and after a medical treatment. In some embodiments, the health state comprises a health state before surgery. In some embodiments, the reference state comprises a health state after surgery. In some embodiments, the health state comprises a menstrual disorder. In some embodiments, the health state comprises endometriosis. In some embodiments, the health state comprises a healthy patient. In some embodiments, the biological material comprises one or more biological materials selected from the group consisting of a RNA, a DNA, a methylated nucleic acid, a miRNA, a protein, a protein-nucleic acid complex, a microorganism, and a mammalian cell type. In some embodiments, constructing the sample menstrualome fingerprint in (d) comprises assaying the extracted biological material from the first sample and the second sample to identify a plurality of biomarkers. In some embodiments, the plurality of menstrualome biomarkers comprise biomarkers that display differential presence or level in cervicovaginal or menstrual fluid as compared to peripheral blood, cervicovaginal tissue, or a longitudinal menstrual sample. In some embodiments, the first sample and reference sample comprise biological material collected at a different time points from the subject. In some embodiments, the sample collector is an intravaginal sample collector. In some embodiments, the sample collector preserves a biological material in an intact state. In some embodiments, the sample collector is capable of absorbing at least 3 ml of fluid. In some embodiments, the sample collector is placed into a buffer subsequent to collecting the sample. In some embodiments, the biological material is DNA and plurality of menstrualome biomarkers comprises methylation status of a plurality of loci. In some embodiments, the biological material is RNA and plurality of menstrualome biomarkers comprises expression level of a plurality of genes. In some embodiments, the biological material is RNA and plurality of menstrualome biomarkers comprises the presence and/or level of a plurality of miRNA. In some embodiments, the biological material is cells and plurality of menstrualome biomarkers measures the presence and/or amount of one or more cell types. In some embodiments, the biological material is DNA and plurality of menstrualome biomarkers measures the presence and/or level of one or more microorganisms. In some embodiments, the biological material is DNA and plurality of menstrualome biomarkers measures the diversity of microorganisms. In some embodiments, the health reference menstrualome fingerprint comprises a principle component analysis, a t-Distributed Stochastic Neighbor Embedding, a heat map, a diversity index, or a combination thereof.

In a further aspect, disclosed herein are methods for preparation of a menstrualome fingerprint. In some embodiments, the method comprises: (a) obtaining a first sample and a second sample from a subject having or suspected to have endometriosis, wherein the first sample and the second sample comprise cervicovaginal or menstrual fluid collected onto an absorbent sample collector; (b) eluting the first sample and the second ample separately from the first and second sample collector into an aqueous buffer; (c) separating a biological material from each of the first sample and the second sample; and (d) constructing a sample menstrualome fingerprint, wherein the sample menstrualome fingerprint comprises the differential of the level and/or presence of a plurality of menstrualome biomarkers in the biological material from the first sample and the second sample. In some embodiments, the biological material comprises one or more biological materials selected from the group consisting of a RNA, a DNA, a methylated nucleic acid, a miRNA, a protein, a protein-nucleic acid complex, a microorganism, and a mammalian cell type. In some embodiments, constructing the sample menstrualome fingerprint in (d) comprises assaying the extracted biological material from the first sample and the second sample to identify a plurality of biomarkers. In some embodiments, the biological material is a miRNA and the plurality of biomarkers comprises a miRNA selected from the group consisting of let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p. The method of claim 93, where the miRNA is selected from the group consisting of miR-1271-5p, miR-4485-3p, miR-125b-2-3p, and miR-410-3p. In some embodiments, the plurality of biomarkers comprises a methylation profile of one or more CpG sites selected from the CpG sites in Table 4. In some embodiments, the microorganism is a bacterium in a genus selected from the group consisting of Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella. In some embodiments, the mammalian cell type is selected from the group consisting of an endothelial cell, an epithelial cell, a leukocyte, a mesenchymal cell, and a combination thereof. In some embodiments, the method further comprises (e) comparing the sample menstrualome fingerprint to a reference menstrualome fingerprint. In some embodiments, the reference menstrualome fingerprint comprises a threshold level or presence of the plurality of menstrualome biomarkers that are associated with a health state. In some embodiments, the health state comprises a health state before surgery. In some embodiments, the reference state comprises a health state after surgery. In some embodiments, the first sample and second sample comprise biological material collected at a different time points from the subject. In some embodiments, the time points are separated by a time period between about 15 minutes and about 30 days. In some embodiments, the time points comprise different days within a menstrual cycle of the subject. In some embodiments, the time points are within a single menstrual cycle. In some embodiments, the time points comprise days in separate menstrual cycles. In some embodiments, the time points are during one or more days of menstruation of the subject. In some embodiments, one time point is during menstruation of the subject and one time point is not during menstruation of the subject. In some embodiments, the sample collector is an intravaginal sample collector. In some embodiments, the sample collector preserves a biological material in an intact state. In some embodiments, the sample collector is capable of absorbing at least 3 ml of fluid. In some embodiments, the sample collector is placed into a buffer subsequent to collecting the sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B illustrate RNA-Seq timecourse data. FIG. 1A shows a Principle component analysis comparing menstrual blood, whole blood, and cervicovaginal fluid. FIG. 1B shows a Principle component analysis comparing menstrual blood and whole blood. FIG. 1C shows a tSNE dimensionality analysis comparing menstrual blood, whole blood, and cervicovaginal fluid. FIG. 1D shows a tSNE dimensionality analysis comparing menstrual blood and whole blood.

FIG. 2A illustrates a timecourse of menstruation. FIG. 2B illustrates changes in gene expression over time for cell specific markers. MUC21 and ALOX12 represent cervicovaginal specific expression. SPRR2F represents ovarian and fallopian tube specific expression. PAEP represents endometrial specific expression. The vertical dashed line represents day 2 of the woman's cycle.

FIG. 3 shows that 11 Kegg pathways are shared between endometriosis and EMT.

FIGS. 4A-4E illustrate bacterial diversity in cervicovaginal fluid and menstrual fluid in “truly healthy,” “suspected unhealthy,” endometriosis, and PCOS patients. A total of 79 patients were analyzed (5 PCOS, 19 with endometriosis, 5 truly healthy, and 50 suspected unhealthy individuals). Box plots represent beta diversity while individual dots represent alpha diversity for a single sample. FIG. 4A illustrates bacterial diversity present in cervicovaginal fluid. FIG. 4B illustrates bacterial diversity present in menstrual fluid. FIG. 4C depicts the bacterial genus with a higher abundance in menstrual fluid than cervicovaginal fluid. FIG. 4D depicts a correlation between the number of overabundant species to the degree of healthiness in patient cohorts. FIG. 4E depicts a comparison of bacterial genus abundance in the healthy cohort in menstrual blood.

FIG. 5 shows a cross-sectional view of an embodiment of a system described herein.

FIGS. 6A-6D illustrate perspective views of an embodiment of the system. FIG. 6A illustrates a full perspective view of an embodiment of the system. FIG. 6B illustrates a perspective view of the upper portion and first end of the central portion of the embodiment of the system of FIG. 6A. FIG. 6C illustrates a perspective view of the bottom of the embodiment of the system of FIG. 6A. FIG. 6D illustrates an additional perspective view of an embodiment of the system of FIG. 6A.

FIGS. 7A-7C illustrate use of an embodiment of the system. FIG. 7A shows the central and lower portions of an embodiment of the system prior to coupling of the upper portion. FIG. 7B shows the embodiment of the system of FIG. 7A following activation of the upper portion. FIG. 7C shows the embodiment of the system of FIG. 7A following activation of the lower portion.

FIGS. 8A-8C illustrate cross-sectional views during use of an embodiment of the system. FIG. 8A shows a cross-sectional view of an embodiment of the system following insertion of a sample collector. FIG. 8B shows a cross-sectional view of the embodiment of the system of FIG. 8A following activation of the upper portion. FIG. 8C shows a cross-sectional view of the embodiment of the system of FIG. 8A following activation of the lower portion.

FIG. 9 is a heatmap schematic showing clustering of the cervicovaginal and menstrual fluid samples over a cycle.

FIG. 10A-10E depict the Kegg pathways regulated by the 5 clusters shown in FIG. 9.

FIG. 11A is a Principle component analysis of the differentially methylated positions menstrual blood and whole blood. FIG. 11B is a tSNE dimensionality analysis of the differentially methylated positions menstrual blood and whole blood.

FIG. 12A displays differentially methylated CpG positions when comparing whole blood and menstrual blood. FIG. 12B displays differentially methylated regions between whole blood and menstrual blood.

FIG. 13A is a Principle component analysis of menstrual blood and whole blood miRNA sequencing. FIG. 13B is a tSNE dimensionality analysis of menstrual blood and whole blood miRNA sequencing.

FIG. 13C depicts a volcano plot illustrating changes in gene expression between all controls and all endometriosis patients. FIG. 13D depicts a volcano plot illustrating changes in gene expression between health patients and endometriosis patients before surgery (left panel) and between healthy patients and endometriosis patients after surgery (right panel).

FIG. 14 depicts the KEGG pathways relevant to differentially regulated miRNAs.

FIG. 15A depicts the signature of differentially present bacterial genuses unique to pre-surgery endometriosis patients. FIG. 15B depicts the signature of differentially present bacterial genuses unique to post-surgery endometriosis patients.

FIG. 16A depicts tSNE clustering of methylation patterns of menstrual blood samples from different patients. FIG. 16B depicts methylation clusters as different cohorts. FIG. 16C depicts the abundance of Lactobacillus in menstrual blood samples per patient.

### DETAILED DESCRIPTION OF THE DISCLOSURE

Non-invasive methods for detection of menstrual disorders, such as early detection of endometriosis, and analysis of menstrual and non-menstrual vaginal fluid are provided herein. In some embodiments, the non-invasive method of detection of endometriosis alleviates the need for a surgical diagnosis, provides the ability to inform clinicians on patient management, and/or allows monitoring of the effectiveness of an intervention. Further provided herein are samples collected from menstrual fluid, systems for collecting samples, and methods for the detection of endometriosis from samples collected from menstrual fluid.

The terminology used herein is for the purpose of describing particular cases only and is not intended to be limiting. In some embodiments, the below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims is drafted to exclude any optional element. As such, this statement is intended to serve as antecedent basis for use of such exclusive terminology as “solely,” “only,” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In some embodiments, in determining whether a number is near to or approximately a specifically recited number, the near or approximating un-recited number is a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein. In some embodiments, the upper and lower limits of these smaller ranges is independently included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

As used herein, the terms “subject,” “individual,” and “patient” are used interchangeably. None of the terms are to be interpreted as requiring the supervision of a medical professional (e.g., a doctor, nurse, physician's assistant, orderly, or hospice worker). As used herein, the subject is any animal, including mammals (e.g., a human or non-human animal). In one embodiment of the methods and compositions provided herein, the mammal is a human. In some embodiments, the subject is a female.

The term “nucleic acid,” as used herein, can generally refer to a polymeric form of nucleotides of any length, either ribonucleotides and/or deoxyribonucleotides. Thus, these terms include, but are not limited to, single-, double-, or multi-stranded DNA or RNA, genomic DNA, complementary DNA (cDNA), mitochondrial DNA (mtDNA), mitochondrial RNA (mtRNA), guide RNA (gRNA), messenger RNA (mRNA), microRNA (miRNA), small interfering RNA (siRNA), transfer RNA (tRNA), ribosomal RNA (rRNA), cell-free DNA (cfDNA), cell-free RNA (cfRNA), DNA-RNA hybrids, or a polymer comprising purine and pyrimidine bases or other natural, chemically or biochemically modified, non-natural, or derivatized nucleotide bases.

As used herein, the term “menstrualome” generally refers to the entirety of: molecules found in the menstrual fluid, molecules isolated from cells found in the menstrual fluid, and cells found in the menstrual fluid, as well as the information that is determined from these molecules and cells. In some cases, molecules are nucleic acids such as DNA or RNA, proteins, metabolites, or a combination thereof. In some cases, cells are endometrial cells, non-endometrial cells such as immune cells and stem cells, bacterial cells, or a combination thereof. In some embodiments, the molecules or cells are from the individual or a vaginal microbiome of the individual. Information determined from molecules include, but are not limited to, for example, the sequence and/or methylation pattern of a DNA sequence, expression level, abundance, or presence of a molecule of interest. Information determined from cells includes but is not limited to, for example, presence or abundance of a cell of interest, including cell surface markers thereof.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Menstrual Fluid Samples

In some embodiments of the methods and systems provided herein, a biological fluid sample, such as a menstrual fluid sample or a sample of another fluid, is collected from a subject using a sample collector which collects fluid from the vaginal cavity. In some embodiments, a sample collector is placed in the vagina or outside the vagina for sample collection. In some embodiments, a sample collector collects a sample by pooling, holding, catching, directing, or absorbing the sample. In some embodiments, a sample collector is absorbent, semi-absorbent, or non-absorbent. In some embodiments, a sample collector is soluble in a buffer. In some embodiments, a sample collector is broken down, for example by exposing the sample collector to an acidic environment, a basic environment, or an enzyme. In some embodiments, sample collectors comprise a pad, a tampon, a vaginal cup, a cervical cap, a menstrual disk, a cervical disk, a sponge, or an interlabial pad. In some embodiments, more than one type of sample collector is used.

In some embodiments of the methods and systems provided herein, a sample collector is left in place for a pre-determined amount of time to collect a biological sample. In some embodiments, at least 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapse. In some embodiments, at most 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapse while the sample collection device is left in place. In some embodiments, about 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapse while the sample collector is left in place.

In some embodiments of the methods and systems provided herein, a sample is collected during the menstrual window (the period) of a subject. In some embodiments, a sample collector is disposable. In some embodiments, a disposable sample collector is discarded or broken down after use. In some embodiments, a disposable sample collector is dissolvable, biodegradable, recyclable, or compostable. In some embodiments, one disposable sample collector is used to collect one sample from one subject. In some embodiments, a sample collector is reusable. In some embodiments, a reusable sample collector is washable, sterilizable, or autoclavable. In some embodiments, reusable sample collector is resistant to degradation, tearing, pore formation, or dissolution. In some embodiments, a reusable sample collector comprises anti-microbial, antibacterial, antiviral, or antifungal properties. In some embodiments, a reusable sample collector is used one or more times to collect one or more samples. In some embodiments, a reusable sample collector is used about 1, 2, 3, 4, 5, 6, 7, 8, 9, 10, 15, 20, 25, 30, or more times to collect one or more biological samples. In some embodiments, a reusable sample collector is used to repeatedly collect biological samples from one subject. In some embodiments, a reusable sample collector is used to collect samples from a plurality of subjects.

In some embodiments of the methods and systems provided herein, one or more samples is collected during one or more periods (menstrual windows) of a subject. In some embodiments, 1 sample is collected during 1 period cycle, 2 samples are collected during 1 period cycle, 3 samples are collected during 1 period cycle, 4 samples are collected during 1 period cycle, more than 4 samples are collected during 1 period cycle, 2 samples are collected during 2 period cycles, 3 samples are collected during 2 period cycles, 4 samples are collected during 2 period cycles, 5 samples are collected during 2 period cycles, 6 samples are collected during 2 period cycles, 7 samples are collected during 2 period cycles, 8 samples are collected during 2 period cycles, more than 8 samples are collected during 2 period cycles, 3 samples are collected during 3 period cycles, 4 samples are collected during 3 period cycles, 5 samples are collected during 3 period cycles, 6 samples are collected during 3 period cycles, 7 samples are collected during 3 period cycles, 8 samples are collected during 3 period cycles, 9 samples are collected during 3 period cycles, 10 samples are collected during 3 period cycles, 11 samples are collected during 3 period cycles, 12 samples are collected during 3 period cycles, more than 12 samples are collected during 3 period cycles, 4 samples are collected during 4 period cycles, 5 samples are collected during 4 period cycles, 6 samples are collected during 4 period cycles, 7 samples are collected during 4 period cycles, 8 samples are collected during 4 period cycles, 9 samples are collected during 4 period cycles, 10 samples are collected during 4 period cycles, 11 samples are collected during 4 period samples, 12 samples are collected during 4 period cycles, 13 samples are collected during 4 period cycles, 14 samples are collected during 4 period cycles, 15 samples are collected during 4 period cycles, 16 samples are collected during 4 period cycles, or more than 16 samples are collected during 4 period cycles. In some embodiments, a plurality of samples is collected during more than 4 period cycles.

In some embodiments, samples are collected outside the menstrual window, e.g., between the time of the subject's periods. In some embodiments, a non-menstrual fluid is collected using the sample collector. In some embodiments, non-menstrual fluid which is collected include vaginal secretions, cervical mucus, cervicovaginal fluid, spotting blood (i.e., from between periods), amniotic fluid, a mucus plug, or other vaginal discharge. In some embodiments, non-menstrual fluid is collected and analyzed using a protocol which is used to collect and analyze menstrual fluid.

In some embodiments, a sample is collected after a menstrual window has closed, e.g., after a period has ended. In some embodiments, a sample is collected on the same day a menstrual window closed. In some embodiments, a sample is collected about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days after a menstrual window has closed. In some embodiments, a sample is collected at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, or at least 30 days after a menstrual window has closed. In some embodiments, a sample is collected not more than 1 day, not more than 2 days, not more than 3 days, not more than 4 days, not more than 5 days, not more than 6 days, not more than 7 days, not more than 8 days, not more than 9 days, not more than 10 days, not more than 11 days, not more than 12 days, not more than 13 days, not more than 14 days, not more than 15 days, not more than 16 days, not more than 17 days, not more than 18 days, not more than 19 days, not more than 20 days, not more than 21 days, not more than 22 days, not more than 23 days, not more than 24 days, not more than 25 days, not more than 26 days, not more than 27 days, not more than 28 days, not more than 29 days, or not more than 30 days after a menstrual window has closed. In some embodiments, a sample is collected between 1 day and 30 days, between 1 day and 25 days, between 1 day and 20 days, between 1 day and 15 days, between 1 day and 10 days, between 1 day and 5 days, between 5 days and 30 days, between 5 days and 25 days, between 5 days and 20 days, between 5 days and 15 days, between 5 days and 10 days, between 10 days and 30 days, between 10 days and 25 days, between 10 days and 20 days, between 10 days and 15 days, between 15 days and 30 days, between 15 days and 25 days, between 15 days and 20 days, between 20 days and 30 days, between 20 days and 25 days, or between 25 days and 30 days after a menstrual window has closed.

In some embodiments, non-menstrual fluid collected between two menstrual windows is collected during various points during the reproductive cycle. Non-menstrual fluid is collected during a pre-ovulation phase, during ovulation, or during a post-ovulation phase. In some embodiments, non-menstrual fluid is collected during a proliferative phase, or during a luteal or secretory phase. In some embodiments, a phase of the reproductive cycle is an abnormal phase. In some embodiments, menstrual fluid and non-menstrual fluid is collected from the same subject.

In some embodiments, a sample is collected between two menstrual windows. In some embodiments, a sample is collected about halfway between two menstrual windows, before the halfway point between two menstrual windows, or after the halfway point between two menstrual windows.

In some embodiments, multiple samples are collected between two menstrual windows. In some embodiments, 2, 3, 4, 5, 6, 7, or 8 samples are collected between two menstrual windows. In some such cases, the multiple samples are collected from different times between the two menstrual windows.

In some embodiments, a sample is collected between two menstrual windows, while a second sample is collected between a second two menstrual windows. In further cases, a third sample is collected between a third two menstrual windows. In a general case, an nth sample is collected between n two menstrual windows, where n is a positive integer which is equal to 1 or more.

In some embodiments, biological samples are collected from a subject both during a menstrual window and between a menstrual window. In some embodiments, a biological sample is collected from a subject during a menstrual window, and a second biological sample is collected from the same subject between two menstrual windows. In some embodiments, a biological sample is collected from a subject during a menstrual window and a second biological sample is collected from the same subject after the end of that menstrual window, and before the next menstrual window. In some embodiments, a biological sample is collected from a subject before the start of a menstrual window, and a second biological sample is collected from the same subject during that menstrual window.

In some embodiments, a volume of fluid, such as menstrual fluid or other fluid collected from a vaginal cavity, is determined using the sample collector. In some embodiments, a volume of menstrual fluid in a sample collector is determined for example by reading graduations on the sample collector. Graduations is at least 0.01 mL, 0.02 mL, 0.03 mL, 0.04 mL, 0.05 mL, 0.06 mL, 0.07 mL, 0.08 mL, 0.09 mL, 0.1 mL, 0.2 mL, 0.3 mL, 0.4 mL, 0.5 mL, 0.6 mL, 0.7 mL, 0.8 mL, 0.9 mL, or 1.0 mL. In some embodiments, a volume of menstrual fluid in a sample collector is determined by measuring the mass of fluid inside the sample collector.

In some embodiments, collected fluid such as menstrual fluid is extracted from the sample collector. In some embodiments, extraction occurs by pouring, pipetting, or suctioning of the fluid, which is appropriate, for example, when the sample collector comprises a menstrual cup or other non-absorbent reservoir. In some embodiments, extraction occurs by dissolving or otherwise breaking down and removing the sample collector from the sample, which is appropriate, for example, when the sample collector comprises a sponge, a tampon, a pad, or another absorbent material. In some embodiments, extraction occurs by squeezing, compressing the sample collector, eluting from the sample collector by placing the collector in a buffer such as an aqueous buffer. In some embodiments, the sample is extracted from the sample collector using the systems, methods, and devices described herein.

Described herein, in certain embodiments, are samples comprising one or more biomarkers, including without limitaitons, nucleic acids, proteins, and cells. In some embodiments, the one or more biomarkers comprise a cell. In some embodiments, cells from a menstrual fluid sample and a preservation solution (e.g., Biomatrica RNAgard). In some embodiments, the sample is an endometrial cell sample comprising one or more endometrial cells. In some embodiments, the sample is an enriched cell sample. In some embodiments, the sample is collected using the systems or devices described herein. In some embodiments, the biomarkers display differential presence or level in cervicovaginal fluid or menstrual fluid as compared to peripheral blood or cervicovaginal tissue.

In some embodiments, the one or more cells is from a biological sample. In some embodiments, the biological sample is taken from a female. In some embodiments, the biological sample is taken from an individual who is suffering from a reproductive disorder, such as for example, chronic pelvic pain, infertility, heavy menstrual bleeding, or a combination thereof. In some embodiments, the individual is a mammal. In some embodiments, the mammal is a human. In some embodiments, the individual is suspected of having endometriosis. In some embodiments, the individual has not received a surgical diagnosis of endometriosis. In some embodiments, the biological sample is taken on a second day of an individual's menstrual cycle. In some embodiments, the biological sample is taken on a day of the individual's menstrual cycle where the individual experiences a heavy flow of menstrual fluid. In some embodiments, the biological sample is taken from the individual prior to administering a treatment, such as a surgery or administration of a therapeutic composition, to the individual. In some embodiments, the treatment, or intervention, is a treatment for endometriosis. In some embodiments, the biological sample is taken from the individual after administering the treatment to the individual. In some embodiments, a first biological sample is taken prior to administering the treatment to the individual and a second biological sample is taken after administering the treatment to the individual. In some embodiments, the method comprises determining a difference in: an expression of one or more microRNAs, a methylation profile of one or more CpG sites selected from the CpG sites in Table 4, a measure of bacterial diversity, or a combination thereof between the first biological sample and the second biological sample.

In some embodiments, the biological sample comprises menstrual fluid. In some embodiments, the biological sample comprises a cervicovaginal fluid, a cervical fluid, or a vaginal fluid. In some embodiments, the biological sample comprises one or more endometrial cells. In some embodiments, the endometrial cells comprises endometrial stromal cells, endometrial epithelial cells, or a combination thereof. In some embodiments, the endometrial cells comprises endometrial stem cells. In some embodiments, the endometrial stem cells comprises menstrual blood mesenchymal stem cells. In some embodiments, the biological sample comprises a non-endometrial cell of the individual. In some embodiments, the non-endometrial cell of the individual comprises a macrophage, a glandular cell, a squamous cell, a cervical columnar cell, a leukocyte, a lymphocyte, a non-endometrial stromal cell, a non-endometrial endothelial cell, a fibroblast, an erythrocyte, a mesenchymal stem cell, an ova, or a combination thereof. In some embodiments, the biological sample comprises one or more spermatozoa.

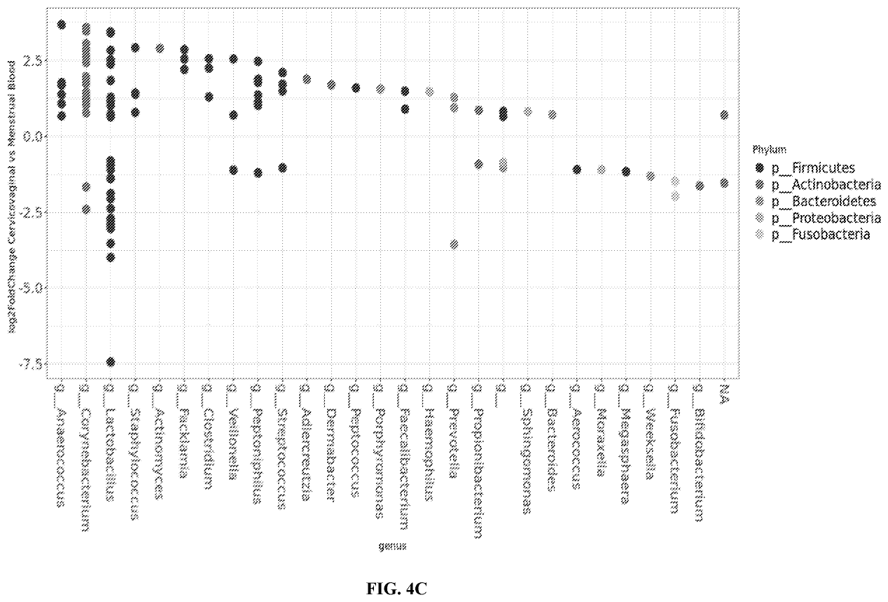

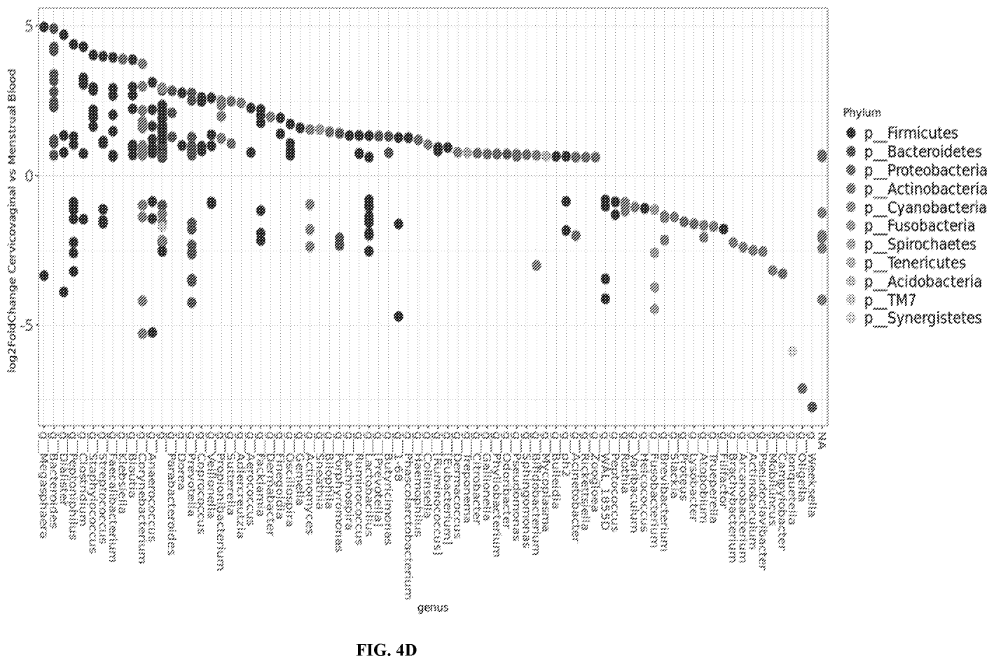

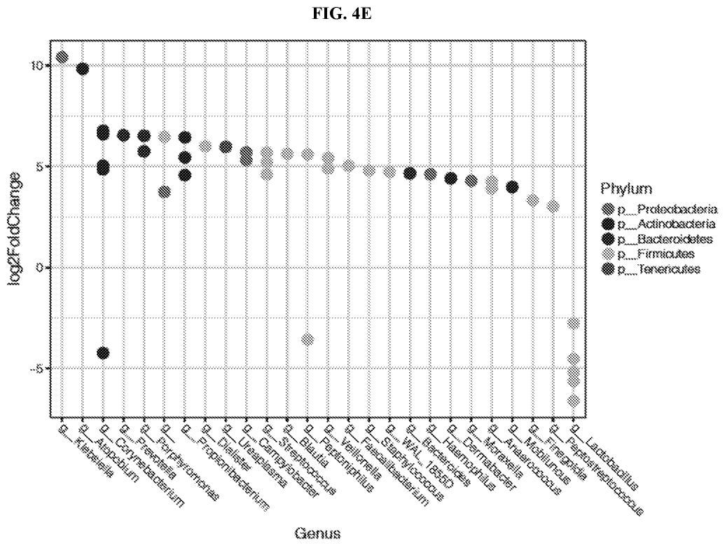

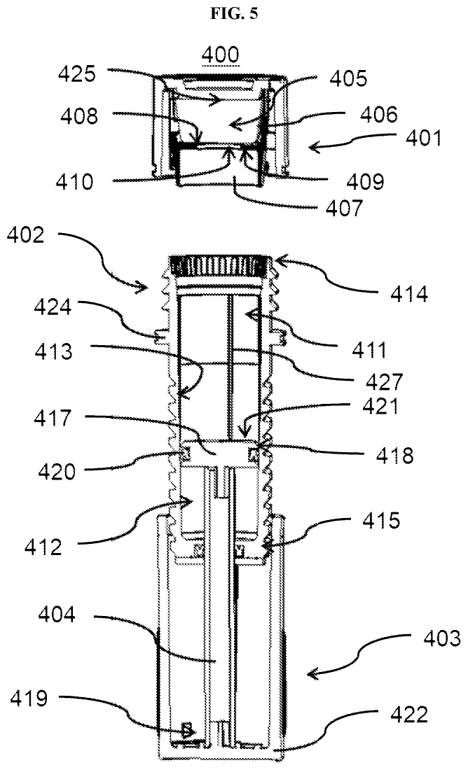

In some embodiments, the biological sample comprises one or more bacterial cells. In some embodiments, the one or more bacterial cells comprise one or more bacterium from the phylum Bacteroidetes, Proteobacteria, Actinobaeria, Cyanobacteria, Fusobacteria, Spirochates, Tenericutes, Acidobacterua, TM7, or Syngerstetes. In some embodiments, the one or more bacterial cells comprise one or more bacteria from the genus Lactobacillus, Gardnerella, Fusobacterium, Staphylococcus, Streptococcus, Atopobium, Mageeibacillus, Mobiluncus, Mycoplasm, Bacteroides, Prevotella, Porphyeromonas, Dialister, Atopobium, Megasphaera, Propionibacterium, Porphyromonas, Dermabacter, Moraxella, Anaerococcus, Peptostreptococcus, Campylobacter, Corynebacterium, Facklamia, Klebsiella, Peptoniphilis, Sneathia, Ureaplasma, Finegoldia, Actinomyces, Clostridium, Veillonella, Peptinophilus, Adlercreurzia, Faecalibacterium, Haemophilus, Sphingomonasm Aerococcus, Weeksella, Biffidobacterium, Blautia, or a combination thereof. In some embodiments, the one or more bacteria comprises a bacteria from a genus described in FIG. 4C, FIG. 4D, FIG. 4E, or a combination thereof. In some embodiments, the one or more bacteria from the genus Lactobacillus is L. acidophilus, L. amylovorus ultunensis, L. coleohominis, L. crispatus, L. fermentum, L. gasseri, L. iners, L. jensenii, L. kitasatonis, L. mucosae, L. paracasei rhamnosus, L. plantarum, L. pontis, L. reuteri frumenti, Lactobacillus sp. 3, Lactobacillus sp. 9, or a combination thereof.

In some embodiments, the one or more bacteria from the genus Gardnerella is Gardnerella vaginalis. In some embodiments, the one or more bacteria from the genus Streptococcus is Streptococcus agalactiae or Streptococcus gallolyticus. In some embodiments, the one or more bacteria from the genus Sneathia is Sneathia sanguinegens. In some embodiments, the one or more bacteria from the genus Mobiluncus is Mobiluncus curtisii, Mobiluncus mulieris, or a combination thereof. In some embodiments, the one or more bacteria from the genus Mageeibacillus is Mageeibacillus indolicus. In some embodiments, the one or more bacteria from the genus Megashaera is Megashaera elsdenii micronuciformis, Megasphaera sp. 1, Megasphaera sp. 2, or a combination thereof. In some embodiments, the one or more bacteria in the genus Dialister is Dialister micraerophilus. In some embodiments, the one or more bacteria from the genus Propionibacterium is Propionibacterium acnes. In some embodiments, the one or more bacteria from the genus Porphyromonas is Porphyromonas somerae. In some embodiments, the one or more bacteria from the genus Dermabacter is Dermabacter vaginalis. In some embodiments, the one or more bacteria from the genus Moraxella is Moraxella catarrhalis.

In some embodiments, the one or more bacteria from the genus Anaerococcus is Anaerococcus tetradius or Anaerococcus prevotii. In some embodiments, the one or more bacteria from the genus Peptostreptococcus is Peptostreptococcus magnus or Peptostreptococcus anaerobius. In some embodiments, the one or more bacteria from the genus Campylobacter is Campylobacter ureolyticus or Camplyobacter fetus. In some embodiments, the one or more bacteria from the genus Cornyebacterium is Corynebacterium amycolatum or Corynebacterium fournierii. In some embodiments, the one or more bacteria from the genus Facklamia is Facklamia hominis or Facklamia massiliensis. In some embodiments, the one or more bacteria from the genus Klebsiella is Klebsiella pneumoniae. In some embodiments, the one or more bacteria from the genus Peptoniphilus is Peptoniphilus harei. In some embodiments, the one or more bacteria from the genus Porphyeromonas is Porphyeromonas asaccharolytica. In some embodiments, the one or more bacteria from the genus Prevotella is Prevotella buccalis, Prevotella amnii, Prevotella bivia, Prevotella disiens, Prevotella melaninogenica, or Prevotella timonensis. In some embodiments, the one or more bacteria from the genus Atopobium is A. deltae, A. minutum, A. parvulum, A. vaginae, or a combination thereof. In some embodiments, the biological sample comprises one or more fungal cells. In some embodiments, the fungal cells is a yeast. In some embodiments, the yeast is a yeast in the genus Candida. In some embodiments, the yeast in the genus Candida is Candida albicans, Candida glabrata, Candida parapsilosis, Candida fomata, or a combination thereof.

In some embodiments, the sample comprises at least one protein or fragment thereof derived from an endometrial cell, a non-endometrial cell from the individual, spermatozoa, bacterial cell, fungal cell, or a combination thereof. In some embodiments, the sample comprises at least one nucleic acid derived from an endometrial cell, a non-endometrial cell from the individual spermatozoa, bacterial cell, fungal cell, or a combination thereof. In some embodiments, the at least one nucleic acid is a cell-free nucleic acid. In some embodiments, the nucleic acid is DNA or RNA. In some embodiments, the RNA is an mRNA, tRNA, rRNA, miRNA, or siRNA. In some embodiments, the nucleic acid is a nucleic acid encoding the at least one protein or fragment thereof described herein.

In some embodiments, the sample comprises a portion of a sample collector. In some embodiments, a portion of the sample collector dissolves or breaks down into the sample. In some embodiments, the sample collector is a tampon, a pad, a vaginal cup, a cervical cap, a menstrual disk, a cervical disk, a sponge, or an interlabial pad. In some embodiments, the tampon is a light absorbency tampon. In some embodiments, the tampon comprises an applicator.

In some embodiments, the volume of the sample is between 2 ml and 15 ml. In some embodiments, the volume of the sample is between about 7 ml and 10 ml. In some embodiments, the volume of the sample is less than 20 ml, less than 15 ml, less than 10 ml, or less than 8 ml. In some embodiments, the volume of the sample is between 1 ml and 4 ml. In some embodiments, the volume of the menstrual fluid in the sample is between 2 ml and 3 ml. In some embodiments, the volume of the menstrual fluid in the sample is less than 5 ml, less than 4 ml, less than 3 ml, less than 2 ml, or less than 1 ml. In some embodiments, the volume of the menstrual fluid in the sample is between 2 ml and 15 ml. In some embodiments, the volume of the sample is between about 7 ml and 10 ml. In some embodiments, the volume of the sample is less than 20 ml, less than 15 ml, less than 10 ml, or less than 8 ml. In some embodiments, the volume of the menstrual fluid in the sample is between 1 ml and 4 ml. In some embodiments, the volume of the menstrual fluid in the sample is between 2 ml and 3 ml. In some embodiments, the volume of the menstrual fluid in the sample is less than 5 ml, less than 4 ml, less than 3 ml, less than 2 ml, or less than 1 ml. In some embodiments, the sample comprises less than 10⁵ cells, less than 10⁶ cells, less than 10⁷ cells, less than 10⁸ cells, or less than 10⁹ cells. In some embodiments, the sample comprises less than 10⁵ endometrial cells, less than 10⁶ endometrial cells, less than 10⁷ endometrial cells, less than 10⁸ endometrial cells, or less than 10⁹ endometrial cells. In some embodiments, the sample comprises greater than 10⁵ cells, greater than 10⁶ cells, greater than 10⁷ cells, greater than 10⁸ cells, or greater than 10⁹ cells. In some embodiments, the sample comprises greater than 10⁵ endometrial cells, greater than 10⁶ endometrial cells, greater than 10⁷ endometrial cells, greater than 10⁸ endometrial cells, or greater than 10⁹ endometrial cells. In some embodiments, the sample comprises less than 10⁵ endothelial cells, less than 10⁶ endothelial cells, less than 10⁷ endothelial cells, less than 10⁸ endothelial cells, or less than 10⁹ endothelial cells. In some embodiments, the sample comprises greater than 10⁵ cells, greater than 10⁶ cells, greater than 10⁷ cells, greater than 10⁸ cells, or greater than 10⁹ cells. In some embodiments, the sample comprises greater than 10⁵ endothelial cells, greater than 10⁶ endothelial cells, greater than 10⁷ endothelial cells, greater than 10⁸ endothelial cells, or greater than 10⁹ endothelial cells. In some embodiments, the sample comprises less than 10⁵ epithelial cells, less than 10⁶ epithelial cells, less than 10⁷ epithelial cells, less than 10⁸ epithelial cells, or less than 10⁹ epithelial cells. In some embodiments, the sample comprises greater than 10⁵ cells, greater than 10⁶ cells, greater than 10⁷ cells, greater than 10⁸ cells, or greater than 10⁹ cells. In some embodiments, the sample comprises greater than 10⁵ epithelial cells, greater than 10⁶ epithelial cells, greater than 10⁷ epithelial cells, greater than 10⁸ epithelial cells, or greater than 10⁹ epithelial cells. In some embodiments, the sample comprises less than 10⁵ leukocytes, less than 10⁶ leukocytes, less than 10⁷ leukocytes, less than 10⁸ leukocytes, or less than 10⁹ leukocytes. In some embodiments, the sample comprises greater than 10⁵ cells, greater than 10⁶ cells, greater than 10⁷ cells, greater than 10⁸ cells, or greater than 10⁹ cells. In some embodiments, the sample comprises greater than 10⁵ leukocytes, greater than 10⁶ leukocytes, greater than 10⁷ leukocytes, greater than 10⁸ leukocytes, or greater than 10⁹ leukocytes. In some embodiments, the sample comprises less than 10⁵ mesenchymal cells, less than 10⁶ mesenchymal cells, less than 10⁷ mesenchymal cells, less than 10⁸ mesenchymal cells, or less than 10⁹ mesenchymal cells. In some embodiments, the sample comprises greater than 10⁵ cells, greater than 10⁶ cells, greater than 10⁷ cells, greater than 10⁸ cells, or greater than 10⁹ cells. In some embodiments, the sample comprises greater than 10⁵ mesenchymal cells, greater than 10⁶ mesenchymal cells, greater than 10⁷ mesenchymal cells, greater than 10⁸ mesenchymal cells, or greater than 10⁹ mesenchymal cells.

In some instances, at least 20%, at least 30%, at least 40%, at least 50%, at least 60%, at least 70%, at least 80%, or at least 90% of the target cells in the sample are intact. In some embodiments, the target cells is endometrial cells. In some embodiments, the target cells is endothelial cells, epithelial cells, leukocytes, mesenchymal cells, or a combination thereof. In some instances, at least 95% of the target cells in the sample are intact. An intact cell is a cell which does not have a ruptured cell membrane. An intact cell is a cell in its native state. An intact cell is a viable cell, wherein the viable cell is cultured in a cell culture.

In some instances, at least 20%, at least 30%, at least 40%, at least 50%, at least 60%, at least 70%, at least 80%, or at least 90% of the target cells in the sample are viable. In some embodiments, the term “viable” means intact, living, and/or capable of proliferation. Viability of a plurality of cells is assessed by measuring membrane permeability, enzymatic activity, metabolic activity, DNA synthesis, membrane potential, proliferation marker expression, or a combination thereof.

In some embodiments, the preservation solution includes Biomatrica LBgard® or Biomatrica RNAgard . In some embodiments, the preservation solution preserves RNA at room temperature for at least 1, 2, 3, 4, 5, 6, 7, 14, or 21 days. In some embodiments, the preservation solution prevents degradation of at least 50%, 60%, 70%, or 80% of the RNA. In some embodiments, the pH range of the preservation solution is from pH 3 to pH 8, or more preferably from pH 3 to pH 6.5. In some embodiments, the preservation solution preserves DNA at room temperature for at least 1, 2, 3, 4, 5, 6, 7, 14, or 21 days. In some embodiments, the preservation solution prevents degradation of at least 50%, 60%, 70%, or 80% of the DNA. In some embodiments, the pH range of the preservation solution is from pH 5 to pH 10, or more preferably from pH 6 to pH 9.

In some embodiments of methods and systems provided herein, the preservation solution preserves the nucleic acid at room temperature for at least 1, 2 , 3, 4, 5, 6, 7, 14, or 21 days. In some embodiments, the preservation solution prevents degradation of at least 50%, 60%, 70%, or 80% of the nucleic acid. In some embodiments, the pH range of the preservation solution is from pH 3 to pH 8, or more preferably from pH 3 to pH 6.5. In some embodiments, the preservation solution preserves RNA at room temperature for at least 1, 2 , 3, 4, 5, 6, 7, 14, or 21 days. In some embodiments, the preservation solution prevents degradation of at least 50%, 60%, 70%, or 80% of the RNA. In some embodiments, the pH range of the preservation solution is from pH 3 to pH 8, or more preferably from pH 3 to pH 6.5. In some embodiments, the preservation solution preserves DNA at room temperature for at least 1, 2, 3, 4, 5, 6, 7, 14, or 21 days. In some embodiments, the preservation solution prevents degradation of at least 50%, 60%, 70%, or 80% of the DNA. In some embodiments, the pH range of the preservation solution is from pH 5 to pH 10, or more preferably from pH 6 to pH 9. In some embodiments, the preservation solution includes Biomatrica LBgard® or Biomatrica RNAgard®.

In some embodiments, the preservation solution comprises a spike-in. As used herein, a “spike-in” is a molecule, such as a nucleic acid, a cell, or a set of molecules or cells added to a sample, wherein the spike-in is used to quantitatively or qualitatively assess or to normalize a sample. In some embodiments, the spike-in comprises a nucleic acid spike-in. In some embodiments, the nucleic acid spike-in comprises a DNA spike-in, an RNA spike-in, a bacterial spike-in, or a combination thereof. In some embodiments, the DNA spike-in comprises a synthetic DNA or a plurality of synthetic DNAs. In some embodiments, the RNA spike-in comprises a synthetic RNA or a plurality of synthetic RNAs. In some embodiments, the RNA spike-in comprises a set of RNA transcripts developed by the External RNA Controls Consortium (ERCC).

In some embodiments, the preservation solution comprises a mucolytic agent. In some embodiments, the mucolytic agent dissociates (e.g., “unclump”) at least a portion of cellular aggregations in the cervicovaginal sample. In some embodiments, the mucolytic comprises acetylcysteine, ambroxol, bromhexine, carbocisteine, domiodol, dornase alfa, eprazinone, erdosteine, letosteine, mannitol, mesna, neltenexine, sobrerol, stepronin, tiopronin, N-acetyl-L-cysteine, L-acetyl cysteine/Liberase™, or a combination thereof.

In some embodiments, the preservation solution comprises an expectorant. In some embodiments, the expectorant comprises althea root, antimony pentasulfide, creosote, guaiacolsulfonate, guaifenesin (+oxomemazine), ipecacuanha, levoverbenone, potassium iodide, senega, tyloxapol, ammonium chloride, or a combination thereof.

In some embodiments, the preservation solution comprises a surfactant. In some embodiments, the surfactant comprises polyoxyethylene glycol octylphenol ethers; polyoxyethylene glycol alkylphenol ethers; polyoxyethylene glycol sorbitan alkyl esters; sorbitan alkyl esters; polyethylene glycol; polypropylene glycol; carboxylates; sulphonates; petroleum sulphonates; alkylbenzenesulphonates; naphthalenesulphonates; olefin sulphonates; alkyl sulphates; sulphates; sulphated esters; sulphated alkanolamides; alkylphenols; ethoxylated aliphatic alcohol; polyoxyethylene surfactants; carboxylic esters; polyethylene glycol esters; anhydrosorbitol esters; glycol esters; carboxylic amide; monoalkanolamine condensates; polyoxyethylene fatty acid amides; quaternary ammonium salts; polyoxyethylene alkyl and alicyclic amines; N,N,N′,N′ tetrakis substituted ethylenediamines; 2-alkyl 1-hydroxethyl 2-imidazolines; or a combination thereof.

In some embodiments, the preservation solution comprises a nuclease. In some embodiments, the nuclease comprises a Benzonase®, DNase I, DNase II, Exonuclease III, Micrococcal Nuclease, Nuclease P1, Nuclease S1, Phosphodiesterase I, Phosphodiesterase II, RNase A, RNase H, RNase T1, or a combination thereof.

In some embodiments, the preservation solution comprises a protease. In some embodiments, the protease comprises adispase II, trypsin, pronase, collagenase 1, collagenase 2, collagenase 3, collagenase 4, hyaluronidase, pepsin, papain, chemotrypsin, chymase, clostripain, complement C1r, complement C1s, complement factor D, complement factor I, cucumisin, dipeptidyl peptidase, elastase, endoproteinase, enterokinase, Factor X Activated, caspase, cathepsin, matrix metalloprotease, or a combination thereof.

In some embodiments, the osmolality of the preservation solution comprises from about 310 to about 410 mOsm kg⁻¹. In some embodiments, the osmolality of the preservation solution comprises from about 95 to about 210 mOsm kg⁻¹.

In some embodiments, the preservation solution does not comprise a fixative. In some embodiments, the fixative comprises an alcohol, an aldehyde, an oxidizing agent, a metallic fixative or a combination thereof. In some embodiments, the alcohol comprises methanol, ethanol, propanol, isopropanol, butanol, or a combination thereof. In some embodiments, the aldehyde comprises formaldehyde, glutaraldehyde, or a combination thereof. In some embodiments, the oxidizing agent comprises an osmium tetraoxide, potassium permanganate, potassium dichromate, or a combination thereof. In some embodiments, the metallic fixative comprises a mercuric chloride, a picric acid, or a combination thereof. In some embodiments, the preservation solution does not comprise an alcohol, an aldehyde, an oxidizing agent, a metallic fixative, or a combination thereof.

In some embodiments, the preservation solution comprises a binding agent. In some embodiments, the binding agent selectively binds to an target cell or a non-target cell of the individual. In some embodiments, the target cell comprises an endothelial cell, an epithelial cell, a leukocyte, a mesenchymal cell, or a combination thereof. In some embodiments, the non-target cell comprises an endothelial cell, an epithelial cell, a leukocyte, a mesenchymal cell, spermatozoa, bacterial cell, fungal cell, or a combination thereof. In some embodiments, the non-target cell comprises different than the target cell. In some embodiments, the binding agent selectively binds to at least one protein or fragment thereof. In some embodiments, the at least one protein or fragment thereof comprises a biomarker of endometriosis. In some embodiments, the binding agent selectively binds to nucleic acid. In some embodiments, the nucleic acid comprises a biomarker of endometriosis. In some embodiments, the binding agent is immobilized, for example, to a bead or to a surface of a component of the systems described herein. In some embodiments, the binding agent is coupled to the bead or the surface of the system. In some embodiments, the binding agent is reversibly or irreversibly coupled to the bead or the surface of the system. In some embodiments, the binding agent comprises a cleavable moiety, for example, a cleavable linker. In some embodiments, the cleavable linker is cleaved photolytically, chemically, thermally, or enzymatically.

In some embodiments, from 0.1 ml to 0.9 ml, from 0.3 ml to 0.7 ml, or from 0.4 ml to 0.6 ml of preservation solution is diluted to form a diluted preservation solution. In some embodiments, the preservation solution comprises Biomatrica LBgard® or Biomatrica RNAgard®. In some embodiments, the preservation solution is diluted in from 4.5 ml to 12.5 ml, from 6.5 ml to 10.5 ml or from 7.5 ml to 9.5 ml of a second solution. In some embodiments, the second solution is distilled water. In some embodiments, a diluted preservation solution is used in the methods and/or systems provided herein. In some embodiments, the diluted preservation solution is added to a sample collector at from 2 ml to 6 ml or from 3 ml to 5 ml of diluted preservation solution per gram of fluid that is absorbed into the sample collector. In some embodiments, a sample collector absorbs up to 6 g of fluid, thus, about 18 ml to about 30 ml of diluted preservation solution is added to the light absorbency tampon. In some embodiments, the diluted preservation solution is added to the sample collector in the system described herein, following the rupture of the disruptable member. Accordingly, as the absorbency of the sample collector increases, the amount of diluted preservation solution to be added increases.

In other embodiments, the preservation solution is not diluted. In some embodiments, the undiluted preservation solution is used in the methods and/or systems provided herein. In some embodiments, the undiluted preservation solution is added to a sample collector at about 3 ml to about 5 ml of undiluted preservation solution per gram of fluid that is absorbed into the sample collector. In some embodiments, a light absorbency tampon absorbs up to 6 g of fluid, thus, about 18 ml to about 30 ml of undiluted preservation solution is added to the sample collector. In some embodiments, the undiluted preservation solution is added to the sample collector in the system described herein, following the rupture of the disruptable member. Accordingly, as the absorbency of the sample collector increases, the amount of undiluted preservation solution to be added increases.

In some embodiments, the binding agent comprises an antibody. In some embodiments, the term “antibody” as used herein refers to immunoglobulin molecules and immunologically active portions of immunoglobulin molecules, i.e., molecules that include an antigen binding site that immunospecifically binds an antigen. In some embodiments, the term also refers to antibodies comprised of two immunoglobulin heavy chains and two immunoglobulin light chains as well as a variety of forms including full length antibodies and portions thereof; including, for example, an immunoglobulin molecule, a polyclonal antibody, a monoclonal antibody, a recombinant antibody, a chimeric antibody, a humanized antibody, a polymer antibody, a CDR-grafted antibody, F(ab)₂, Fv, scFv, IgGΔCH₂, F(ab)₂, scFv2CH₃, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies). In some embodiments, the antibody comprises a polymer antibody. In some embodiments, the antibodies is configured to selectively bind target cells over non-target cells. In some embodiments, the antibodies is configured to selectively bind non-target cells over target cells.

Described herein, in certain embodiments, are methods of preserving cells from a menstrual fluid sample. In some embodiments, the cells comprises endometrial cells or non-endometrial cells. In some embodiments, the method comprises disposing the menstrual fluid in a preservation solution to form a mixture of the menstrual fluid sample and the preservation solution. In various embodiments, disposing the menstrual fluid sample in a preservation solution to form the mixture comprises placing a sample collector into a first central cavity of a system wherein the sample collector is compressed or squeezed, for example, to remove at least a portion of the sample from the sample collector. In some embodiments, the sample collector comprises a tampon, a pad, a menstrual disk, a cervical cup, a cervical disk, a sponge, an interlabial pad, or another suitable sample collector. In some instances, placing the sample collector into the first central cavity is carried out by the individual from whom the menstrual fluid sample was collected. In some instances, placing the sample collector into the first central cavity is carried out by a medical professional, such as an obstetrician or nurse. Upon compression of the sample collector, endometrial cells in the menstrual fluid sample is broken or sheared such that contents of the endometrial cells (e.g., nucleic acids) are released into the mixture. In some instances, compression of the sample collector in a manner that compresses the sample collector is carried out by the individual from whom the menstrual fluid sample was collected. In some instances, compression of the sample collector is carried out by at a laboratory or other location which processes the sample collector for assaying the collected sample.

In some embodiments, the methods described herein comprises contacting the cells in the menstrual fluid sample with an antibody that binds to a cell surface antigen of a target cell in the cells in the menstrual fluid sample. In some embodiments, when the target cell comprises an endothelial cell, the cell surface antigen comprises CD31/PECAM-1, CD34, CD36/SR-B3, CD39, CD44, CD47, CD54/ICAM-1, CD61, CD62E, CD62P, CD80, CD86, CD93, CD102, CD105, CD106, CD112, CD117, ESAM, Endomucin, CXCL16, CD121a, CD141, CD142, CD143, CD144, CD146, CD147, CD151, CD160, CD201, CD213a, CD248, CD309, ADAMs 8, ADAMs 9, ADAMs 10, ADAMs 11, ADAMs 12, ADAMs 13, ADAMs 14, ADAMs 15, ADAMs 16, ADAMs 17, ADAMs 33, ADAMTS-13, ADAMTS-18, VWF, TEM8, NOTCH, or KLF4. In some embodiments, when the target cell is an epithelial cell, the cell surface antigen is Epithelial cell adhesion molecule (EpCAM), E-cadherin, or CD326. In some embodiments, when the target cell is a leukocyte, the cell surface antigen is CD45. In some embodiments, when the target cell is a mesenchymal cell, the cell surface antigen is N-cadherin, OB-cadherin, alpha-5 beta-1 integrin, alpha-V beta-6 integrin, or syndecan-1.

In some embodiments, the methods described herein comprises contacting the cells in the menstrual fluid sample with an antibody that binds to a target cell in the cells in the menstrual fluid sample. In some embodiments, the antibody comprises a monoclonal antibody. In some embodiments, the antibody is attached to a solid support. In some embodiments, the solid support is a bead. In some embodiments, the bead is a magnetic bead. In some embodiments, the antibody is conjugated with a detectable marker. In some embodiments, the detectable marker comprises an optically detectable marker. In some embodiments, the optically detectable marker comprises a fluorophore. In some embodiments, the fluorophore comprises a dye, for example, fluorescein isothiocyanate (FITC), phycoerythrin (PE), allophycocyanin (APC), or peridinin chlorophyll protein (PerCP). In some embodiments, the fluorophore comprises a fluorescent protein, for example, green fluorescent protein (GFP), enhanced green fluorescent protein (EGFP), cyan fluorescent protein (CFP), yellow fluorescent protein (YFP), red fluorescent protein (RFP), or mCHERRY. In some embodiments, the fluorophore emit a wavelength of light of from 355 nm to 650 nm.

In some embodiments, the methods described herein further comprise enriching a sample for at least one target cell, thereby producing an enriched cell sample. In some embodiments, the cell sample comprises a menstrual fluid cell sample. In some embodiments, the enriched sample comprises an enriched menstrual fluid cell sample. In some embodiments, the at least one target cell comprises an endometrial cell. In some embodiments, the at least one target cell comprises an endothelial cell, an epithelial cell, a leukocyte, a mesenchymal cell, or a combination thereof. In some embodiments, at least one non-target cell comprises an endothelial cell, an epithelial cell, a leukocyte, a mesenchymal cell, or a combination thereof. In some embodiments, the endothelial cell comprises an endometrial endothelial cell.

In some embodiments, enriching for the at least one target cell comprises increasing an amount of at least one target cell in the enriched cell sample relative to an amount of the at least one target cell in the cell sample prior to enrichment. Enriching for the at least one target cell comprises increasing a ratio of at least one target cell to at least one non-target cell in the enriched cell sample relative to a ratio of the at least one target cell to at least one non-target cell in the cell sample prior to enrichment. In some embodiments, enriching for the at least one target cell comprises isolating the at least one target cell bound to at least one antibody. In some embodiments, the isolated at least one target cell bound to the antibody comprises the enriched cell sample. Enriching for the at least one target cell comprises removing at least one non-target cell from the cell sample, wherein the at least one non-target cell is bound by at least one antibody. In some embodiments, the cell sample following removing of at least one non-target cell thereby produces the enriched cell sample. Isolating a target cell bound to an antibody or a non-target cell bound to an antibody comprises the use of flow cytometry. Isolating a target cell bound to an antibody or a non-target cell bound to an antibody comprises the use of fluorescence activated cell sorting (FACS), magnetic activated cell sorting (MACS), or the combination thereof.

Described herein, in certain embodiments, are methods of preserving nucleic acids, proteins and/or metabolites from a menstrual fluid sample In some embodiments, the method comprises disposing the menstrual fluid in a preservation solution to form a mixture of the menstrual fluid sample and the preservation solution, where the preservation solution preserves the integrity of the nucleic acid (DNA or RNA) or one or more metabolites or protein. In various embodiments, disposing the menstrual fluid sample in a preservation solution to form the mixture comprises placing a sample collector into a first central cavity of a system wherein the sample collector is compressed or squeezed, for example, to remove at least a portion of the sample from the sample collector. In some embodiments, the sample collector comprises a tampon, a pad, a menstrual disk, a cervical cup, a cervical disk, a sponge, an interlabial pad, or another suitable sample collector. In some instances, placing the sample collector into the first central cavity is carried out by the individual from whom the menstrual fluid sample was collected. In some instances, placing the sample collector into the first central cavity is carried out by a medical professional, such as an obstetrician or nurse. In some instances, compression of the sample collector in a manner that compresses the sample collector is carried out by the individual from whom the menstrual fluid sample was collected. In some instances, compression of the sample collector is carried out by at a laboratory or other location which processes the sample collector for assaying the collected sample.

In some embodiments, once collected in the systems described herein, the sample collected is kept at room temperature for at least 1 day, 2 days, 3 days, 4 days, 5 days, 6 days, 7 days, 8 days, 9 days, 10 days, 11 days, 12 days, 13 days, or 14 days. In some embodiments, the sample is kept at room temperature for up to 2 weeks. In some embodiments, the method comprises shipping the mixture. In some embodiments, the incubating occurs before shipping the mixture, during the shipping of the mixture, after the mixture has been delivered, or any combination thereof. In some embodiments, the mixture is shipped, for example, to a testing facility or to a healthcare provider's office.

In some embodiments, the preservation solution comprises 1-methyl-3-carboxyethyl-imidazolium bromide, 1-hexyl-3-methyimidazolium bromide, 1-octyl-3-methylimidazolium bromide, 1-decyl-3-methylimidazolium bromide, or 1-(2-hydroxyethyl)-3-methylimidazolium bromide. In some embodiments, the 1-methyl-3-carboxyethyl-imidazolium bromide, 1-hexyl-3-methyimidazolium bromide, 1-octyl-3-methylimidazolium bromide, 1-decyl-3-methylimidazolium bromide, or 1-(2-hydroxyethyl)-3-methylimidazolium bromide is present in the preservation solution at a concentration of about 0.1% to 10% (w/v). In some embodiments, the preservation solution further comprises a precipitating agent, a lower alcohol, a chaotrope, a chelating agent, a reducing agent, a pH buffer, water, a surfactant, or a combination thereof. In some embodiments, the preservation solution comprises at least one of: the precipitating agent, the lower alcohol, and the chaotrope. In some embodiments, the preservation solution comprises at least one of: the chelating agent, the reducing agent, the pH buffer.

In some embodiments, the preservation solution comprises the surfactant. In some embodiments, the surfactant is a detergent. In some embodiments, the precipitating agent is 5-(4-dimethyl)amino benzylidene rhodanine, sulfosalicyclic acid, lithium chloride, or lithium hydroxide. In some embodiments, the lower alcohol comprises methanol, ethanol, n-propanol, isopropanol, n-butanol, or isobutanol (2-methylpropan-1-ol). In some embodiments, the chaotrope comprises guanidine hydrochloride, guanidine thiocyanate, potassium thiocynanate, sodium thiocyanate, or urea. In some embodiments, the chelating agent comprises diethylenetriaminepentaacetic acid (DTPA), ethyl enediaminetetraacetic acid (EDTA), ethylene glycol tetraacetic acid (EGTA), trans-1,2-diaminocyclohexane-N,N,N′,N′-tetraacetic acid (CDTA), 1,2-bis(2-aminophenoxy)ethane-N,N,N′,N′-tetraacetic acid (BAPTA), 1,4,7,10-tetraazacyclododecane-1,4,7,10-tetraacetic acid (DOTA), N-(2-hydroxyethyl)ethylenediamine-N,N′,N′-triacetic acid, or nitrilotriacetic acid (NTA). In some embodiments, the reducing agent comprises 2-mercaptoethanol, thiosulfate, TCEP (tris-(2-carboxyethyl) phosphine), dithiothreitol, or dithioerythritol. In some embodiments, the pH buffer comprises citric acid, tartaric acid, malic acid, sulfosalicylic acid, sulfoisophtalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), IVIES (2-(N-morpholino)ethanesulfonic acid), MOPS (3-(N-morpholine)propanesulfonic acid), MOPSO (3-morpholine-2-hydroxypropanesulfonic acid), PIPES (1-4-piperazinediethanesulfonic acid), TAPS (N-[tris(hydroxymethyl)methyl]-3-aminopropanesulfonic acid), TAPSO (2-hdyroxy-3-[tris(hdyroxymethyl)methylamino]-1-propanesulfonic acid), TES (N-[tris(hydroxymethyl)methyl]-2-aminoethanesulfonic acid), bicine (N,N-Bis(2-hdyroxyethyl)glycine), tricine (N-[Tris(hydroxymethyl)methyl]glycine), tris (tris(hydroxymethyl)aminomethane), or bis-tris (2-[Bis(2-hdyroxyethyl)amino]-2-(hdyroxymethyl)-1,3-propanediol). In some embodiments, the detergent comprises Triton® X-100, Nonidet® P40, a Brij® detergent, a Tomamine® ethoxylated amine detergent, and a Surfonic® detergent. In some embodiments, the detergent comprises bis-(2-hydroxyethyl) isodecyloxypropylamine, poly (5) oxyethylene isodecyloxypropylamine, bis-(2-hydroxyethyl) isotridecyloxypropylamine, poly (5) oxyethylene isotridecyloxypropyl amine, bis-(2-hydroxyethyl) linear alkyloxypropylamine, bis (2-hydroxyethyl) soya amine, poly (15) oxyethylene soya amine, bis (2-hydroxyethyl) octadecylamine, poly (5) oxyethylene octadecylamine, poly (8) oxyethylene octadecylamine, poly (10) oxyethylene octadecylamine, poly (15) oxyethylene octadecylamine, bis (2-hydroxyethyl) octadecyloxypropylamine, bis-(2-hydroxyethyl) tallow amine, poly (5) oxyethylene tallow amine, poly (15) oxyethylene tallow amine, poly (3) oxyethylene 1 ,3 diaminopropane, bis (2-hydroxyethyl) coco amine, bis-(2-hydroxyethyl) isodecyloxypropylamine, poly (5) oxyethylene isodecyloxypropylamine, bis-(2-hydroxyethyl) isot decyloxypropylamine, poly (5) oxyethylene isotridecyloxypropyl amine, bis-(2-hydroxyethyl) linear alkyloxypropylamine, bis (2-hydroxyethyl) soya amine, poly (15) oxyethylene soya amine, bis (2-hydroxyethyl) octadecylamine, poly (5) oxyethylene octadecylamine, poly (8) oxyethylene octadecylamine, poly (10) oxyethylene octadecylamine, poly (15) oxyethylene octadecylamine, bis (2-hydroxyethyl) octadecyloxypropylamine, bis-(2-hydroxyethyl) tallow amine, poly (5) oxyethylene tallow amine, poly (15) oxyethylene tallow amine, poly (3) oxyethylene 1,3 diaminopropane, or bis (2-hydroxethyl) coco amine. In some embodiments, the surfactant comprises any surfactant from the Tween® family of surfactants.

In some embodiments, the preservation solution comprises at least one of: a preservation agent, a dissociation agent, or a combination thereof. In some embodiments, the preservation agent comprises a zwitterionic compound, an osmoprotectant, an apoptosis inhibitor, a non-reducing sugar or polyol, a disaccharide derivative, a chelating agent, a pH buffer, a phosphatase inhibitor, a protease inhibitor, or a combination thereof. In some embodiments, the dissociation agent comprises a mucolytic, an expectorant, a surfactant, a nuclease, a protease, or a combination thereof. In some embodiments, the preservation solution further comprises a spike-in. In some embodiments, the preservation solution consists essentially of: a zwitterionic compound, an osmoprotectant, an apoptosis inhibitor, a non-reducing sugar or polyol, a chelating agent, a pH buffer, a phosphatase inhibitor, a protease inhibitor, a mucolytic, an expectorant, a surfactant, a nuclease, a protease, a spike-in, or any combination thereof. In some embodiments, the preservation solution comprises an agent for selective lysis of non-target cells but not of target cells in the sample. In some embodiments, the preservation solution comprises an agent for selective lysis of a cell that is not an endometrial cell. In some embodiments, the agent for selective lysis comprises the dissociation agent. In some embodiments, the agent for selective lysis comprises the nuclease, the protease, or a combination thereof. In some embodiments, the preservation solution selectively lyses about 10%, 20%, 30%, 40%, 50%, 60%, 70%, 80%, 90%, 95%, or 99% of the non-target cells in the sample. In some embodiments, the preservation solution selectively lyses about 10%, 20%, 30%, 40%, 50%, 60%, 70%, 80%, 90%, 95%, or 99% of the cells which are not target cells in the sample. In some embodiments, the preservation solution further comprises a binding agent.

In some embodiments, the preservation solution comprises a zwitterionic compound. In some embodiments, the zwitterionic compound comprises a betaine or a betaine analog. In some embodiments, the zwitterionic compound comprises trimethylamino N-oxide (TMAO). In some embodiments, the zwitterionic compound comprises N-Tri s(hydroxymethyl)methyl-2-aminoethanesulfonic acid; 3-(N,N-bis[2-hydroxyethyl]amino)-2-hydroxypropanesulphonic acid; 3-(N-morpholino)propanesulfonic acid, 4-(2-hydroxyethyl)-1-piperazineethanesulfonic acid; Tris(hydroxymethyl)aminomethane; piperazine-N,N′-bis(2-ethanesulfonic acid); 2-(N-Morpholino)ethanesulfonic acid hydrate; N,N-Bis(2-hydroxyethyl)-2-aminoethanesulfonic acid; N-[Tris(hydroxymethyl)methyl]glycine; 3-((3-acrylamidopropyl)-dimethylammonio)-propane-1-sulfonate; hydroxyectoine; ectoine; homoectoine; L-carnitine; sarcosine; N,N-Dimethylglycine triethylammonium acetate; glycerol phosphate; tricine; pentaerythritol; N-ethyl-N,N-bis-(2-hydroxyethyl)ammonium-N-4-butyl sulfonate; 3-morpholino-2-hydroxypropanesulfonic acid; 4-(2-ethoxy-2-oxoethyl)-4-ethylmorpholin-4-ium bromide; N-(2-ethoxy-2-oxoethyl)-3-hydroxy-N,N-bis(2-hydroxyethyl)propan-1-aminium bromide; 2-ethoxy-N,N,N-triethyl-2-oxoethanaminium bromide; 2-((3-hydroxypropyl)dimethylammonio)acetate; 2-((2-hydroxypropyl)dimethylammonio) acetate; 2-(2-(hydroxymethyl)-1-methylpiperidinium-1-yl)acetate; 2-((2-hydroxyethyl)dimethylammonio)acetate; 2-((2,3-dihydroxypropyl) dimethylammonio)acetate; 1-(2-ethoxy-2-oxoethyl)-4-hydroxy-1-methylpiperidinium bromide; 2-(4-hydroxy-1-methylpiperidinium-1-yl)acetate; 2-ethoxy-N-(2-(2-hydroxyethoxy)ethyl)-N,N-dimethyl-2-oxoethanaminium bromide; 2-((2-(2-hydroxyethoxy)ethyl)dimethylammonio)acetate; 2-(bis(2-hydroxyethyl)-(methyl)ammonio)acetate; 4-(2-hydroxyethyl)-4-methyl-2-oxomorpholin-4-ium bromide; 2-(bis(2-hydroxyethyl)-(methyl)ammonio)acetate; 2-(4-(2-hydroxyethyl)morpholino-4-ium)acetate; 4-(2-ethoxy-2-oxoethyl)-4-methylmorpholin-4-ium bromide; 1-(2-ethoxy-2-oxoethyl)-1-methylpyrrolidinium bromide; 2-(benzyl(2-hydroxy-ethyl)(methyl)ammonio)acetate; 3-(2,3-dihydroxypropyl)-1-methyl-1H-imidazol-3-ium chloride; 1,3-dimethyl-1H-imidazol-3-ium methyl sulfate; N-benzyl-2-ethoxy-N,N-dimethyl-2-oxoethanaminium bromide; 1-(2-ethoxy-2-oxoethyl)-1-methylpiperidi-nium bromide; N-(2-ethoxy-2-oxoethyl)-N,N-dimethylbenzenaminium bromide; 1-(2-ethoxy-2-oxoethyl)-3-hydroxy-1-methylpiperidinium bromide; 3-(2-(2-hydroxyethoxy)ethyl)-1-methyl-1H imidazol-3-ium chloride; 3-(2-(2-(2-hydroxyethoxy)ethoxy)ethyl)-1-methyl-1H-imidazol-3-ium chloride; 1-methyl-3-tetradecyl-1H-imidazol-3-ium bromide; N-(2-ethoxy-2-oxoethyl)-N,N-dimethylcyclo-hexanaminium bromide; 3-((2-hydroxy-ethyl)dimethyl-ammonio)pro-panoate; or any combination thereof. In some embodiments, the zwitterionic compound comprises a polyzwitterion. In some embodiments, the polyzwitterion comprises carboxybetaine methacrylate-1; carboxybetaine methacrylate-1-tertiary amine; carboxybetaine methacrylate-2; carboxybetaine acrylamide-2; carboxybetaine acrylamide-2-ethyl ester; carboxybetaine acrylamide-2-RGD; carboxybetaine diacrylamide crosslinker; glycine betaine; poly-sulfobetaine; or any combination thereof.

In some embodiments, the preservation solution comprises an osmoprotectant. In some embodiments, the osmoprotectant comprises trimethylammonium acetate; glycerol phosphate; diglycerol phosphate, N-(2-hydroxy-1,1-bis(hydroxymethyl)ethyl)glycine; 3-(N-morpholino)-2-hydroxypropanesulfonic acid; pentaerythritol; glyceric acid; malic acid; tartaric acid; lactic acid; glycolic acid; 2-hydroxybutyric acid; 3-hydroxybutyric acid; 4-amino-3-hydroxybutyric acid; 3-(1-azoniabicyclo[2.2.2]oct-1-yl)propane-1-sulfonate; 1-(2-carboxylatoethyl)-1-azabicyclo[2.2.2]octan-1-ium; or any combination thereof.

In some embodiments, the preservation solution comprises an apoptosis inhibitor. In some embodiments, the apoptosis inhibitor comprises PERK-eIF2-α inhibitor, ASK1 inhibitor, NRF2-KEAP1 inhibitor, JNK inhibitor, p38 MAP kinase inhibitor, IRE1 inhibitor, GSK3 inhibitor, PIK3 pathway inhibitor, MEK inhibitor, calpain inhibitor, caspase-1 inhibitor, or any combination thereof.

In some embodiments, the preservation solution comprises a non-reducing sugar or polyol. In some embodiments, the non-reducing sugar or polyol comprises glycol, glycerol, erythritol, threitol, arabitol, xylitol, ribitol, adonitol, mannitol, sorbitol, galactitol, fucitol, iditol, inositol, adonitol, sucralfate, sucrose octasulfate, sucrose, trehalose, or any combination thereof. In some embodiments, the preservation solution comprises a disaccharide derivative. In some embodiments, the disaccharide derivative comprises sucralose, trichloronated maltose, or a combination thereof.

In some embodiments, the preservation solution comprises a chelating agent. In some embodiments, the chelating agent comprises diethylenetriaminepentaacetic acid (DTPA); ethylenediaminetetraacetic acid (EDTA); ethylene glycol tetraacetic acid (EGTA); trans-1,2-diaminocyclohexane-N,N,N′,N′-tetraacetic acid (CDTA); 1,2-bis(2-aminophenoxy)ethane-N,N,N′,N′-tetraacetic acid (BAPTA); 1,4,7,10-tetraazacyclododecane-1,4,7,10-tetraacetic acid (DOTA); N-(2-hydroxyethyl)ethylenediamine-N,N′,N′-triacetic acid; sodium gluconate; nitrilotriacetic acid (NTA); or a combination thereof.

In some embodiments, the preservation solution comprises a pH buffer. In some embodiments, the pH buffer comprises citric acid; tartaric acid; malic acid; sulfosalicylic acid; sulfoisophthalic acid; oxalic acid; borate; CAPS (3-(cyclohexylamino)-1-propanesulfonic acid); CAPSO (3-(cyclohexylamino)-2-hydroxy-1-propanesulfonic acid); EPPS (4-(2-hydroxyethyl)-1-piperazinepropanesulfonic acid); HEPES (4-(2-hydroxyethyl)piperazine-1-ethanesulfonic acid); IVIES (2-(N-morpholino)ethanesulfonic acid); MOPS (3-(N-morpholino)propanesulfonic acid); MOPSO (3-morpholino-2-hydroxypropanesulfonic acid); PIPES (1,4-piperazinediethanesulfonic acid); TAPS (N[tris(hydroxymethyl)methyl]-3-aminopropanesulfonic acid); TAPSO (2-hydroxy-3-[tris(hydroxymethyl)methylamino]-1-propanesulfonic acid); TES (N-[tris(hydroxymethyl)methyl]-2-aminoethanesulfonic acid); bicine (N,N-bis(2-hydroxyethyl)glycine); tricine (N-[tris(hydroxymethyl)methyl]glycine); tris (tris(hydroxymethyl)aminomethane); bis-tris (2-[bis(2-hydroxyethyl)amino]-2-(hydroxymethyl)-1,3-propanediol); or a combination thereof.

In some embodiments, the preservation agent comprises a phosphatase inhibitor. In some embodiments, the phosphatase inhibitor comprises beta-Glycerophosphate, aprotinin, bestatin, EDTA, leupeptin, pepstatin A, or a combination thereof.

In some embodiments, the preservation agent comprises a protease inhibitor. In some embodiments, the protease inhibitor comprises (2R)-2-Mercaptomethyl-4-methylpentanoyl-beta-(2-naphthyl)-Ala-Ala Amide; 2-Antiplasmin; 3,4-Dichloroisocoumarin; 4-(2-Aminoethyl) benzenesulfonyl fluoride hydrochloride; 5-(R,S)-T-trans-Cinnamido-7-methyl-4-oxo-octanoyl-L-prolyl-L-proline ; a1-Antchymotrypsin; a1-Antitrypsin; a2-Antiplasmin; a2-Macroglobulin; Antithrombin III; Aprotinin; Bromoenol lactone; BTEE; C1 Esterase inhibitor; Chymostatin; Complement C1 esterase inhibitor; Dichloromethylenediphosphonic acid disodium salt; Diisopropyl fluorophosphate; e-Amino-n-caproic acid; Ecotin; EDTA; Eglin C fragment 60-63 methyl ester; Gabexate mesylate; Histatin 5; Ile-Pro-Ile; Isoamylphosphonyl-Gly-L-Pro-L-Ala; Leupeptin; N a-p-Tosyl-L-lysine chloromethyl ketone hydrochloride; N-Acetyl-eglin C; N-Tosyl-L-phenylalanine chloromethyl ketone; p-Chloromercuribenzoic acid Free Acid; Phenylmethylsulfonyl fluoride; Trypsin Inhibitor; Trypsin-chymotrypsin inhibitor; Z-L-Phe chloromethyl ketone; Boc-Asp(OMe)-fluoromethyl ketone; Z-Ala-Glu(OMe)-Val-Asp(OMe)-fluoromethyl ketone; Antipain dihydrochloride from microbial source protease inhibitor; CA-074 methyl ester; Calpain Inhibitor I; Calpain Inhibitor II; Cystatin; E-64 protease inhibitor; Leupeptin trifluoroacetate salt; a2-Macroglobulin; Procathepsin B; Z-Leu-Leu-Leu-fluoromethyl ketone; Z-Phe-Phe-fluoromethyl ketone; or a combination thereof.

### Preperation of a Menstrualome Fingerprint

Also described herein are methods for preparation of a menstrualome fingerprint. In some embodiments, the menstrualome comprises the entirety of molecules found in the menstrual fluid, molecules isolated from cells found in the menstrual fluid, and cells found in the menstrual fluid, as well as the information that is determined from these molecules and cells. In some embodiments, the sample menstrualome fingerprint comprise the differential of the level and/or presence of a plurality of menstrualome biomarkers in the biological material from the first sample and the second sample. In some embodiments, the menstrualome fingerprint comprises a biological signature of biomarkers that are specific to a specific state in the menstrual cycle. In some embodiments, the menstrual fingerprint represents a specific genomic profile of menstrual fluid suitable for diagnostic development. In some embodiments, the menstrualome fingerprint acts as a non-invasive biopsy for collection of endometrial tissue. In some embodiments, the menstrualome biomarkers comprises a matrix of biological signatures from the endometrial tissue shed during menstruation.

In certain aspects, the systems and methods described herein comprise a method for preparation of a menstrualome fingerprint. In certain embodiments, the method for preparation of a menstrualome fingerprint comprises obtaining a sample using the methods and systems described herein. In certain embodiments, the method for preparation of a menstrualome fingerprint comprises obtaining a first sample and a second sample using the methods and systems described herein. In some embodiments, the methods comprise extracting a biological material from the sample or samples obtained herein into an aqueous buffer. In some embodiments, the methods comprises separating a biological material from the sample or samples obtained. In some embodiments, the methods comprise constructing a menstrualome fingerprint.

In some embodiments, the sample menstrualome fingerprint comprises the differential of the level and/or presence of a plurality of menstrualome biomarkers in the biological material from the first sample and the second sample. In some embodiments, the sample menstrualome fingerprint comprises the differential of the level and/or presence of a plurality of menstrualome biomarkers in the biological material from the first sample and/or the second sample as compared to a reference menstrualome fingerprint. In some embodiments, the sample menstrualome fingerprint comprises the level and/or presence of a plurality of menstrualome biomarkers in the biological material from the first sample. In some embodiments, the sample menstrualome fingerprint is compared to a reference fingerprint. In some embodiments, the biomarkers as described herein display differential presence or level in cervicovaginal fluid or menstrual fluid as compared to peripheral blood or cervicovaginal tissue.

In some embodiments, the method comprises obtaining a first sample and a second sample from a subject, wherein the first sample and the second sample comprise cervicovaginal or menstrual fluid collected onto a first and a second absorbent sample collector; eluting the first sample and the second sample separately from the first and the second sample collector into an aqueous buffer; separating a biological material from each of the first sample and the second sample; and constructing a sample menstrualome fingerprint, wherein the sample menstrualome fingerprint comprises the differential of the level and/or presence of a plurality of menstrualome biomarkers in the biological material from the first sample and the second sample. In some embodiments, the method comprises obtaining a first sample and a second sample from a subject, wherein the first sample and the second sample comprise cervicovaginal or menstrual fluid collected onto a first and a second absorbent sample collector; eluting the first sample and the second sample separately from the first and the second sample collector into an aqueous buffer; separating a biological material from each of the first sample and the second sample; constructing a sample menstrualome fingerprint, wherein the sample menstrualome fingerprint comprises the differential of the level and/or presence of a plurality of menstrualome biomarkers in the biological material from the first sample and/or the second sample as compared to a reference menstrualome fingerprint. In some embodiments, the methods comprise: obtaining a first sample from a subject, wherein the first sample comprise cervicovaginal or menstrual fluid collected onto an absorbent sample collector; eluting the first sample from the sample collector into an aqueous buffer; separating a biological material from the first sample; constructing a sample menstrualome fingerprint, wherein the sample menstrualome fingerprint comprises the level and/or presence of a plurality of menstrualome biomarkers in the biological material from the first sample; and comparing the sample menstrualome fingerprint to a reference fingerprint. In some embodiments, the sample is collected and/or preserved using methods and devices described herein. In some embodiments, the subject has or is suspected of having endometriosis.

In some embodiments, the subject is a female. In some embodiments, the subject is suffering from chronic pelvic pain, infertility, heavy menstrual bleeding, or a combination thereof. In some embodiments, the subject is a mammal. In some embodiments, the mammal is a human. In some embodiments, the subject is suspected of having endometriosis. In some embodiments, the subject has not received a surgical diagnosis of endometriosis. In some embodiments, the subject has a family history of endometriosis. In some embodiments, the endometriosis is deep infiltrating endometriosis (DIE), superficial peritoneal endometriosis (SPE), or ovarian endometriomas (OE).

In some embodiments, the sample comprises any biological material or sample as described herein, including menstrual fluid samples and cervicovaginal fluid samples. In certain embodiments, the biological material comprises one or more of the biological materials described herein. In some embodiments, the biological material includes, without limitations, a RNA, a DNA, a miRNA, a protein, a microorganism, and a mammalian cell. In some embodiments, the biological material is RNA and plurality of menstrualome biomarkers comprises expression level of a plurality of genes. In some embodiments, the biological material is RNAs and plurality of menstrualome biomarkers comprises the presence and/or level of a plurality of miRNAs. In some embodiments, the biological material is cells and plurality of menstrualome biomarkers measures the presence and/or amount of one or more cell types. In some embodiments, the biological material is DNA and plurality of menstrualome biomarkers measures the presence and/or level of one or more microorganisms. In some embodiments, the biological material is DNA and plurality of menstrualome biomarkers measures the diversity of microorganisms.

In certain embodiments, the methods described herein comprise a method or assay to separate or analyze the extracted biological material from the sample or samples described herein. In certain embodiments, the methods described herein comprise separating a biological material from the sample. In certain embodiments, separating a biological material from the sample comprises isolating the biological material from the sample using the methods or assays described herein. In certain embodiments, the biological material comprises nucleic acid, protein, a cell, or a combination thereof.

In some embodiments, the method or assay comprises isolation of the nucleic acid, protein, or a combination thereof from the cervicovaginal sample described herein. In some embodiments, the method or assay comprises isolation of the nucleic acid, protein, or a combination thereof from the sample described herein. In various embodiments, aliquots of the sample are created. In some embodiments, the method or assay comprises isolation of the nucleic acids from a first aliquot of the sample and isolation of proteins from a second aliquot of the sample. Isolation of the nucleic acids, proteins, or a combination thereof from the sample comprises lysis of the cells in the sample; extraction of the nucleic acids, proteins, or a combination thereof from the sample; and/or purification of the extracted nucleic acids, extracted proteins, or a combination thereof.

In some embodiments, the method or assay comprises lysis of the cells in the sample. In some embodiments, the lysis is a chemical lysis, mechanical lysis, or a combination thereof. In some embodiments, chemical lysis comprises the addition of a lytic enzyme, a chaotropic agent, a detergent, or a combination thereof to the sample. In some embodiments, mechanical lysis comprises homogenizing, ultrasonicating, shearing, or shocking the cells. In some embodiments, the shocking comprises osmotic shock. In some embodiments, lysis results in release of the nucleic acids and proteins of the cell. In some embodiments, the method or assay comprises purification of the nucleic acids, proteins, or a combination thereof in the sample.

In some embodiments, the method or assay comprises extraction of the nucleic acids, proteins, or a combination thereof from the sample. In some embodiments, the nucleic acids is DNA, RNA, or combination thereof. In some embodiments, the RNA comprises mRNA, tRNA, rRNA, miRNA, siRNA, or a combination thereof. Extraction comprises organic phase extraction. In some embodiments, the method or assay comprises purification of the extracted nucleic acids, extracted proteins, or a combination thereof.

In some embodiments, the method or assay comprises sequencing the nucleic acid from the sample or the enriched sample. In some embodiments, the sequencing is whole-genome sequencing or whole-exome sequencing. In some embodiments, the sequencing is high-throughput sequencing. In some embodiments, the nucleic acid is sequences to a depth of at least 5×, 10×, 20×, 30×, 40×, 50×, 60×, 70×, 80×, 90×, 100×, 150×, 200×, 250×, 300×, or more than 300× coverage. In some embodiments, the sequencing is targeted sequencing, wherein one or more pre-selected nucleic acid targets are sequenced. In some embodiments, the one or more pre-selected nucleic acid targets is one or more biomarkers specific to endometriosis. In some embodiments, the sequencing comprises sequencing of 16S rRNA or 16S rDNA. In some embodiments, the method or assay comprises bisulfite treatment prior to the sequencing. In some embodiments, the methods or assays described herein comprise determining a methylation status of a nucleic acid in a nucleic acid sequence (i.e., methylated or not methylated). In some embodiments, the nucleic acid is a cytosine. In some embodiments, the methods or assays described herein comprise determining a methylation pattern of a nucleic acid sequence.

In some embodiments, the method or assay comprises determining, from a biological sample of the individual, an expression level of one or more microRNAs (miRs) selected from the group consisting of: miR-1271-5p, miR-4485-3p, miR-125b-2-3p, and miR-410-3p. In some embodiments, the method or assay comprises determining, from a biological sample of the individual, an expression level of one or more microRNAs (miRs) selected from the group consisting of: miR-23b-3p, miR-30a-3p/5p, and miR-34a-5p. In some embodiments, the method or assay comprises determining, from the biological sample, an expression level of one or more miRs selected from the group consisting of: let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-12′7-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p. In some embodiments, the method or assay comprises determining, from the biological sample of the individual, an expression level of one or more microRNA that is, or that is predicted to, regulate an expression of at least one gene involved in at least one KEGG pathway. In some embodiments, the KEGG pathway is: “ECM-receptor,” “Adherens junction,” “Proteoglycans in cancer,” “TGF-beta signaling,” “Hippo signaling,” “MicroRNAs in cancer,” “Pathways in cancer,” “Hepatitis B,” “Glioma,” “Chronic myeloid leukemia,” “Bladder cancer,” or a combination thereof. In some embodiments, the at least one KEGG pathway is involved with Wnt/JNK/VEGF signaling.

In some embodiments, the method or assay comprises determining a methylation profile of one or more CpG sites selected from the CpG sites in Table 4.

In some embodiments, the menstrualome footprint comprises a determining a measure of bacterial diversity in the biological sample. In some embodiments, the measure of bacterial diversity is an amount of at least 1, 2, 3, 4, 5, 6, 7, 8, 9, 10, or more bacteria.

In some embodiments, the biological sample comprises one or more bacterial cells. In some embodiments, the one or more bacterial cells comprises one or more bacterium from the phylum Bacteroidetes, Proteobacteria, Actinobaeria, Cyanobacteria, Fusobacteria, Spirochates, Tenericutes, Acidobacterua, TM7, or Syngerstetes. In some embodiments, the one or more bacterial cells comprises one or more bacteria from the genus Lactobacillus, Gardnerella, Fusobacterium, Staphylococcus, Streptococcus, Atopobium, Mageeibacillus, Mobiluncus, Mycoplasm, Bacteroides, Prevotella, Porphyeromonas, Dialister, Atopobium, Megasphaera, Propionibacterium, Porphyromonas, Dermabacter, Moraxella, Anaerococcus, Peptostreptococcus, Campylobacter, Corynebacterium, Facklamia, Klebsiella, Peptoniphilis, Sneathia, Ureaplasma, Finegoldia, Actinomyces, Clostridium, Veillonella, Peptinophilus, Adlercreurzia, Faecalibacterium, Haemophilus, Sphingomonasm Aerococcus, Weeksella, Biffidobacterium, Blautia, or a combination thereof. In some embodiments, the one or more bacteria comprises a bacteria from a genus described in FIG. 4C, FIG. 4D, FIG. 4E, or a combination thereof. In some embodiments, the measure of bacterial diversity comprises a ratio of at least one first bacterium to at least one second bacterium.

In some embodiments, the measure of bacterial diversity comprises a diversity index. In some embodiments, the diversity index comprises a Shannon diversity index, a Simpson diversity index, or a Berger-Parker diversity index. In some embodiments, the bacterial diversity measures diversity in bacterial species, genera, families, functional types, or haplotypes. In some embodiments, the bacterial diversity is determined by sequencing. In some embodiments, the sequencing comprises Sanger sequencing or high-throughput sequencing. In some embodiments, the sequencing identifies a species of the bacteria in the biological sample. In some embodiments, the sequencing identifies an abundance of the species of the bacteria. In some embodiments, the sequencing is sequencing of a 16S rRNA or a portion thereof.

In some embodiments, the biomarkers display differential presence of level in cervicovaginal or menstrual fluid between one or more health states. In some embodiments, the biomarkers display differential presence or level in cervicovaginal fluid or menstrual fluid as compared to peripheral blood or cervicovaginal tissue.

In some embodiments, the method further comprises comparing the sample menstrualome fingerprint to a reference menstrualome fingerprint. In some embodiments, the reference menstrualome fingerprint comprises a threshold level or presence of the plurality of menstrualome biomarkers that are associated with a health state. In some embodiments, the health reference menstrualome fingerprint comprise a principle component analysis, a t-Distributed Stochastic Neighbor Embedding, a heat map, a diversity index, classical, metric and non-metric multidimensional scaling (MDS), a diffusion map, receiver operator curves, k means clustering, discriminative model building, multivariate logistic regression with stepwise feature selections, trees, random forests, and principal component analysis. In some embodiments, the reference state comprises a health state before or after surgery. In some embodiments, the reference state comprises a patient without endometriosis. In some embodiments, the reference state comprises a healthy subject. In some embodiments, the healthy subject is a subject that does not have a family history of endometriosis. In some embodiments, the healthy subject is a subject that does not suffer from or is not suspected of having a reproductive disorder, including, but not limited to, polycystic ovarian syndrome (PCOS), endometriosis, or a combination thereof. In some embodiments, the healthy subject is a subject with a family history of the reproductive disorder.

In some embodiments, the first sample and the second sample comprise any sample or biological sample as described herein. In some embodiments, the first sample and second sample comprises biological material collected at a different time points from the subject. In some embodiments, the time points are separated by a time period between about 15 minutes and about 30 days. In some embodiments, the time points are separated by a time period of at least about 5, 10, 15, 20, 25, 30, 35, 40, 45, 50, 55, or 60 minutes. In some embodiments, the time points are separated by a time period of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the time points are separated by a time period of at least about 1, 2, 3, 4, 5, 6, 7, 8, 9, 10, 11, or 12 months. In some embodiments, the time points are separated by a time period of no more than about 5, 10, 15, 20, 25, 30, 35, 40, 45, 50, 55, or 60 minutes. In some embodiments, the time points are separated by a time period of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the time points are separated by a time period of no more than about 1, 2, 3, 4, 5, 6, 7, 8, 9, 10, 11, or 12 months. In some embodiments, the time points are separated by a time period of no more than about 1, 2, 3, 4, 5, 6, 7, 8, 9, 10, or more than 10 years

In some embodiments, the time points comprise different days within a menstrual cycle of the subject. In some embodiments, a normal menstrual cycle occurs approximately every month and comprises the shedding of the lining of the uterus through the vagina. In some embodiments, normal menstrual flow lasts approximately 4 or5 days, lasts up to 7 days, and occurs every 21 to 35 days. In some embodiments, the time points are within a single menstrual cycle. In some embodiments, the time points comprise days in separate menstrual cycles. In some embodiments, the time points are during one or more days of menstruation of the subject. In some embodiments, one time point is during menstruation of the subject and one time point is not during menstruation of the subject. In some embodiments, at least one time point is during a heavy bleeding day. In some embodiments, at least one time point is during a light bleeding day. In some embodiments, at least one time point is not during a bleeding day. In some embodiments, at least one time point is during ovulation.

In some embodiments, the two or more health states comprise before and after a medical treatment. In some embodiments, the medical treatment is a surgery, such as a surgery to treat endometriosis or other menstrual disorders. In some embodiments, the health state comprises a health state before or after surgery. In some embodiments, the health state is chronic pelvic pain, infertility, heavy menstrual bleeding, eating disorders; extreme weight loss; excessive exercise; polycystic ovary syndrome (PCOS); ovarian cysts; premature ovarian failure; breast cancer; ovarian cancer; infertility; diminished ovarian reserve; chronic or frequent urinary tract infections; ectopic pregnancy; heart disease; type 1 diabetes; type 2 diabetes; an autoimmune condition such as lupus, multiple sclerosis, or rheumatoid arthritis; pelvic inflammatory disease (PID); fibroids (e.g., uterine fibroids); adenomyosis; cervical cancer; endometrial cancer; uterine cancer; bacterial vaginosis, chlamydia, gonorrhea, genital herpes, hepatitis, human immunodeficiency virus, acquired immunodeficiency syndrome, human papillomavirus, syphilis, trichomoniasis, or infection of the cervix or endometrium, or a combination thereof. In some embodiments, the health state comprises a menstrual disorder. In some embodiments, the endometriosis is deep infiltrating endometriosis (DIE), superficial peritoneal endometriosis (SPE), or ovarian endometriomas (OE).

In some embodiments, the reference footprint comprises the expression level of the one or more microRNAs, a methylation profile, a measure of bacterial diversity, or a combination thereof of the individuals of known endometriosis state.

In some embodiments, the method or assay further comprises generating a report based on a biomarker or biomarker signature. In some embodiments, the biomarker or biomarker signature comprises the expression level of the one or more miRNAs relative to a reference expression level, the methylation profile of the one or more genomic regions, the measure of bacterial diversity, or a combination thereof.

In some embodiments, a method or assay for classifying or detecting endometriosis in an individual includes determining from a biological sample (e.g., a menstrual fluid sample) of the individual an expression level of one or more microRNAs (miRs). In some instances, the biological sample or the menstrual fluid sample is collected on a first, second, third, fourth, fifth, sixth, and/or seventh day of the individual's menstrual cycle. In certain instances, the biological sample or the menstrual fluid sample is collected on the second day of the individual's menstrual cycle.

In some embodiments, the method or assay further includes determining from the biological sample of the individual an expression level of two, three, or more microRNAs (miRs). In some embodiments, the miRs is selected from miR-1271-5p, miR-4485-3p, miR-125b-2-3p, and/or miR-410-3p. In some embodiments, the miRs comprise intracellular miRs, extracellular miRs, or intracellular and extracellular miRs. In certain embodiments, the miRs are isolated from cells in the biological sample. In various embodiments, the miRs are isolated from a non-cellular portion of the biological sample. In some embodiments, the miRs are isolated from the total biological sample (e.g., from both intracellular and extracellular portions of the biological sample). In some embodiments, the miRs are assessed or detected by any suitable method. In some embodiments, the miRs is assessed or detected using sequencing.

In some embodiments, the biological sample includes menstrual fluid, cervicovaginal fluid, or both. In some embodiments, the biological sample is disposed in a sample collector as provided herein. In some embodiments, the sample collector is a pad, a tampon, a vaginal cup, a cervical cap, a menstrual disk, a cervical disk, a sponge, an interlabial pad, or a combination thereof.

### Detection of a Disorder

Described herein, in certain embodiments, are methods of or assays for detecting a disorder in an individual. Described herein, in certain embodiments, are methods of or assays for detecting endometriosis in an individual. In some embodiments, the individual is a female. In some embodiments, the individual is suffering from chronic pelvic pain, infertility, heavy menstrual bleeding, or a combination thereof. In some embodiments, the individual is a mammal. In some embodiments, the mammal is a human. In some embodiments, the individual is suspected of having endometriosis. In some embodiments, the individual has not received a surgical diagnosis of endometriosis. In some embodiments, the individual has a family history of endometriosis. In some embodiments, the endometriosis is deep infiltrating endometriosis (DIE), superficial peritoneal endometriosis (SPE), or ovarian endometriomas (OE). In some embodiments, the methods or assays described herein has a false discovery rate of less than 10%, 9%, 8%, 7%, 6%, 5%, 4%, 3%, 2%, or 1%. In some embodiments, the methods or assays described herein has a false discovery rate of 5% or less. In some embodiments, methods or assays for classifying or detecting endometriosis in an individual are provided herein. In some embodiments, the methods or assays has a specificity of at least 80%, 85%, 90%, 95%, 96%, 97%, 98%, 99%, or 100%.

In some embodiments, the methods or assays describe herein comprise removing the sample described herein from a system described herein. In some embodiments, the sample is removed from the system through a port located on the system. In some embodiments, the sample is removed from the system via a syringe inserted through the port. In some embodiments, from about 2 ml to about 4 ml of sample are removed from the system. In certain embodiments, from about 1 ml to about 5 ml of sample are removed from the system.

In some embodiments, the method or assay comprises isolation of a biomarker, including without limitations, a nucleic acid, protein, or cell, from the sample. In some embodiments, the method or assay comprises isolation of the nucleic acid, protein, or a combination thereof from the sample. In various embodiments, aliquots of the sample are created. In some embodiments, the method or assay comprises isolation of the nucleic acids from a first aliquot of the sample and isolation of proteins from a second aliquot of the sample. Isolation of the nucleic acids, proteins, or a combination thereof from the sample comprises lysis of the cells in the sample; extraction of the nucleic acids, proteins, or a combination thereof from the sample; and/or purification of the extracted nucleic acids, extracted proteins, or a combination thereof. In some embodiments, the biomarkers display differential presence or level in cervicovaginal fluid or menstrual fluid as compared to peripheral blood or cervicovaginal tissue.

In some embodiments, the method or assay comprises lysis of the cells in the sample. In some embodiments, the lysis is a chemical lysis, mechanical lysis, or a combination thereof. Chemical lysis comprises the addition of a lytic enzyme, a chaotropic agent, a detergent, or a combination thereof to the sample. Mechanical lysis comprises homogenizing, ultrasonicating, shearing, or shocking the cells. In some embodiments, the shocking comprises osmotic shock. In some embodiments, lysis result in release of the nucleic acids and proteins of the cell. In some embodiments, the method or assay comprises purification of the nucleic acids, proteins, or a combination thereof in the sample.

In some embodiments, the method or assay comprises extraction of the nucleic acids, proteins, or a combination thereof from the sample. In some embodiments, the nucleic acids is DNA, RNA, or combination thereof. In some embodiments, the RNA comprises mRNA, tRNA, rRNA, miRNA, siRNA, or a combination thereof. Extraction comprises organic phase extraction. In some embodiments, the method or assay comprises purification of the extracted nucleic acids, extracted proteins, or a combination thereof.

In some embodiments, the method or assay comprises sequencing the nucleic acid from the sample or the enriched sample. In some embodiments, the sequencing is whole-genome sequencing or whole-exome sequencing. In some embodiments, the sequencing is high-throughput sequencing. In some embodiments, the nucleic acid is sequences to a depth of at least 5×, 10×, 20×, 30×, 40×, 50×, 60×, 70×, 80×, 90×, 100×, 150×, 200×, 250×, 300×, or more than 300× coverage. In some embodiments, the sequencing is targeted sequencing, wherein one or more pre-selected nucleic acid targets are sequenced. In some embodiments, the one or more pre-selected nucleic acid targets is one or more biomarkers specific to endometriosis. In some embodiments, the sequencing comprises sequencing of 16S rRNA or 16S rDNA. In some embodiments, the method or assay comprises bisulfite treatment prior to the sequencing. In some embodiments, the methods or assays described herein comprise determining a methylation status of a nucleic acid in a nucleic acid sequence (i.e., methylated or not methylated). In some embodiments, the nucleic acid is a cytosine. In some embodiments, the methods or assays described herein comprise determining a methylation pattern of a nucleic acid sequence.

In some embodiments, the method or assay comprises determining, from a biological sample of the individual, an expression level of one or more microRNAs (miRs) selected from the group consisting of: miR-1271-5p, miR-4485-3p, miR-125b-2-3p, and miR-410-3p. In some embodiments, the method or assay comprises determining, from a biological sample of the individual, an expression level of one or more microRNAs (miRs) selected from the group consisting of: miR-23b-3p, miR-30a-3p/5p, and miR-34a-5p. In some embodiments, the method or assay comprises determining, from the biological sample, an expression level of one or more miRs selected from the group consisting of: let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-12′7-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p. In some embodiments, the method or assay comprises determining, from the biological sample of the individual, an expression level of one or more microRNA that is, or that is predicted to, regulate an expression of at least one gene involved in at least one KEGG pathway. In some embodiments, the KEGG pathway is: “ECM-receptor,” “Adherens junction,” “Proteoglycans in cancer,” “TGF-beta signaling,” “Hippo signaling,” “MicroRNAs in cancer,” “Pathways in cancer,” “Hepatitis B,” “Glioma,” “Chronic myeloid leukemia,” “Bladder cancer,” or a combination thereof. In some embodiments, the at least one KEGG pathway is involved with Wnt/JNK/VEGF signaling.

In some embodiments, the method or assay comprises comparing the expression level to a reference expression level of the one or more miRs. In some embodiments, the comparing comprises performing a differential expression analysis. A machine learning algorithm is used for the differential expression analysis. In some embodiments, the reference expression level is obtained from a healthy subject. In some embodiments, the healthy subject is a subject that does not suffer from endometriosis or is not suspected of having endometriosis. In some embodiments, the healthy subject is a subject that does not have a family history of endometriosis. In some embodiments, the healthy subject is a subject that does not suffer from or is not suspected of having a reproductive disorder, including, but not limited to, polycystic ovarian syndrome (PCOS), endometriosis, or a combination thereof. In some embodiments, the healthy subject is a subject with a family history of the reproductive disorder. In some embodiments, an increased or decreased expression level of the one or more miRs relative to the reference expression level indicates that the subject has endometriosis.

In some embodiments, the method or assay comprises determining a methylation profile of one or more CpG sites selected from the CpG sites in Table 4.

In some embodiments, the method or assay comprises determining a measure of bacterial diversity in the biological sample. In some embodiments, the measure of bacterial diversity is an amount of at least 1, 2, 3, 4, 5, 6, 7, 8, 9, 10, or more bacteria. In some embodiments, the biological sample comprises one or more bacterial cells. In some embodiments, the one or more bacterial cells comprises one or more bacterium from the phylum Bacteroidetes, Proteobacteria, Actinobaeria, Cyanobacteria, Fusobacteria, Spirochates, Tenericutes, Acidobacterua, TM7, or Syngerstetes. In some embodiments, the one or more bacterial cells comprises one or more bacteria from the genus Lactobacillus, Gardnerella, Fusobacterium, Staphylococcus, Streptococcus, Atopobium, Mageeibacillus, Mobiluncus, Mycoplasm, Bacteroides, Prevotella, Porphyeromonas, Dialister, Atopobium, Megasphaera, Propionibacterium, Porphyromonas, Dermabacter, Moraxella, Anaerococcus, Peptostreptococcus, Campylobacter, Corynebacterium, Facklamia, Klebsiella, Peptomphilis, Sneathia, Ureaplasma, Finegoldia, Actinomyces, Clostridium, Veillonella, Peptinophilus, Adlercreurzia, Faecalibacterium, Haemophilus, Sphingomonasm Aerococcus, Weeksella, Biffidobacterium, Blautia, or a combination thereof. In some embodiments, the one or more bacteria comprises a bacteria from a genus described in FIG. 4C, FIG. 4D, FIG. 4E, or a combination thereof., L. reuteri frumenti, Lactobacillus sp. 3, Lactobacillus sp. 9, or a combination thereof. In some embodiments, the measure of bacterial diversity is a ratio of at least one first bacterium to at least one second bacterium.

In some embodiments, the measure of bacterial diversity is a diversity index. In some embodiments, the diversity index is a Shannon diversity index, a Simpson diversity index, or a Berger-Parker diversity index. In some embodiments, the bacterial diversity measures diversity in bacterial species, genera, families, functional types, or haplotypes. In some embodiments, the bacterial diversity is determined by sequencing. In some embodiments, the sequencing is Sanger sequencing or high-throughput sequencing. In some embodiments, the sequencing identifies a species of the bacteria in the biological sample. In some embodiments, the sequencing identifies an abundance of the species of the bacteria. In some embodiments, the sequencing is sequencing of a 16S rRNA or a portion thereof. In some embodiments, an increase in bacterial diversity in the biological sample relative to a reference bacterial diversity indicates that the subject has endometriosis. In some embodiments, the reference bacterial diversity level is a bacterial diversity in a healthy individual. In some embodiments, the healthy subject is a subject that does not suffer from endometriosis or is not suspected of having endometriosis. In some embodiments, the healthy subject is a subject that does not suffer from or is not suspected of having a reproductive disorder, including, but not limited to, polycystic ovarian syndrome (PCOS), endometriosis, or a combination thereof. In some embodiments, the healthy subject is a subject with a family history of the reproductive disorder.

In one example, the bacteria is Propionibacterium acnes. In some embodiments, an increase in the abundance of P. acnes relative to a reference P. acnes abundance indicates that the subject has endometriosis. In some embodiments, an increase in an abundance of P. acnes at least 5 times greater, at least 10 times greater, or at least 15 times greater than a reference P. acnes abundance indicates that the subject has endometriosis. In some embodiments, the reference P. acnes abundance is an abundance of P. acnes in a healthy subject. In some embodiments, the healthy subject is a subject that does not suffer from endometriosis or is not suspected of having endometriosis. In some embodiments, the healthy subject is a subject that does not suffer from or is not suspected of having a reproductive disorder, including, but not limited to, polycystic ovarian syndrome (PCOS), endometriosis, or a combination thereof. In some embodiments, the healthy subject is a subject with a family history of the reproductive disorder.

In some embodiments, the method or assay comprises applying a classifier algorithm (or classifier) to the expression level of the one or more microRNAs, a methylation profile, a measure of bacterial diversity, or a combination thereof from the biological sample from the individual, thereby generating a classification of the individual. In some embodiments, the classification is selected from the group consisting of: likely endometriosis and not likely endometriosis. In some embodiments, the classification of likely endometriosis is selected from the group consisting of: high likelihood of endometriosis, moderate likelihood of endometriosis, and low likelihood of endometriosis. In some embodiments, the classification is a numerical score quantifying the likelihood that the individual has endometriosis. In some embodiments, the method or assay comprises using a machine learning model to generate the classifier algorithm. Generating the classifier algorithm comprises the use of training data from individuals of known endometriosis status (e.g., individuals diagnosed with endometriosis or individuals diagnosed without endometriosis). In some embodiments, the training data comprises the expression level of the one or more microRNAs, a methylation profile, a measure of bacterial diversity, or a combination thereof of the individuals of known endometriosis state. In some embodiments, the classifier algorithm comprises a decision tree, random forest, Bayesian network, support vector machine, neural network, or logistic regression algorithm. In some embodiments, the classifier is a random forest classifier. In some embodiments, the random forest classifier comprises at least 10, 20, 50, 100, 1000, or 5000 decision trees. A machine learning model is used for differential expression analysis.

In some embodiments, the method or assay further comprises generating a report based on a biomarker or biomarker signature. In some embodiments, the biomarker or biomarker signature comprises the expression level of the one or more miRNAs relative to a reference expression level, the methylation profile of the one or more genomic regions, the measure of bacterial diversity, or a combination thereof. In some embodiments, the method or assay further comprises transmitting the report to a health practitioner. In some embodiments, the report contains a recommendation for administering an intervention to the individual. In some embodiments, the intervention comprises a surgical intervention, a therapeutic intervention, or a combination thereof. In some embodiments, the surgical intervention comprises surgical removal of at least a part of an endometriosis lesion, hysterectomy, salpingo-oophorectomy, presacral neurectomy, or laparoscopic uterine nerve ablation. In some embodiments, the therapeutic intervention comprises administration of a therapeutic agent. In some embodiments, the therapeutic agent is a hormone, a hormone agonist, hormone antagonist, aromatase inhibitor, an anti-inflammatory therapy, acetyltransferase, histone deacetylase inhibitor, phosphodiesterase inhibitor, or a combination thereof. In some embodiments, the hormone is a synthetic hormone. In some embodiments, the hormone is estrogen, progestin, progesterone, androgen, gonadotropin-releasing hormone (Gn-RH), or a combination thereof. In some embodiments, the hormone agonist is a gonadotropin-releasing hormone (Gn-RH) agonist. In some embodiments, the hormone antagonist is a gonadotropin-releasing hormone (Gn-RH) antagonist. In some embodiments, the therapy is a birth control comprising the hormone. In some embodiments, the anti-inflammatory therapy is an NSAID, JNK inhibitor, TNF inhibitor, an interleukin (IL) inhibitor, or a combination thereof.

In some embodiments, the method or assay further comprises administering an intervention to the individual. In some embodiments, the intervention is determined from the report. In some embodiments, the intervention is determined in the absence of the report. In some embodiments, the intervention comprises a surgical intervention, a therapeutic intervention, or a combination thereof. In some embodiments, the surgical intervention comprises surgical removal of at least a part of an endometriosis lesion, hysterectomy, salpingo-oophorectomy, presacral neurectomy, or laparoscopic uterine nerve ablation. In some embodiments, the therapeutic intervention comprises administration of a therapeutic agent. In some embodiments, the therapeutic agent is a hormone, a hormone agonist, hormone antagonist, aromatase inhibitor, an anti-inflammatory therapy, acetyltransferase, histone deacetylase inhibitor, phosphodiesterase inhibitor, or a combination thereof. In some embodiments, the hormone is a synthetic hormone. In some embodiments, the hormone is estrogen, progestin, progesterone, androgen, gonadotropin-releasing hormone (Gn-RH), or a combination thereof. In some embodiments, the hormone agonist is a gonadotropin-releasing hormone (Gn-RH) agonist. In some embodiments, the hormone antagonist is a gonadotropin-releasing hormone (Gn-RH) antagonist. In some embodiments, the therapy is a birth control comprising the hormone. In some embodiments, the anti-inflammatory therapy is an NSAID, INK inhibitor, TNF inhibitor, an interleukin (IL) inhibitor, or a combination thereof. In some embodiments, the method or assay further comprises an assessment of the success, likelihood of success, incomplete success or failure, likelihood of failure of the intervention.

In some embodiments, a method or assay for classifying or detecting endometriosis in an individual includes determining from a biological sample (e.g., a menstrual fluid sample) of the individual an expression level of one or more microRNAs (miRs). In some instances, the biological sample or the menstrual fluid sample is collected on a first, second, third, fourth, fifth, sixth, and/or seventh day of the individual's menstrual cycle. In certain instances, the biological sample or the menstrual fluid sample is collected on the second day of the individual's menstrual cycle.

In some embodiments, the method or assay further includes determining from the biological sample of the individual an expression level of two, three, or more microRNAs (miRs). In some embodiments, the miRs is selected from miR-1271-5p, miR-4485-3p, miR-125b-2-3p, and/or miR-410-3p. In some embodiments, the miRs comprises intracellular miRs, extracellular miRs, or intracellular and extracellular miRs. In certain embodiments, the miRs is isolated from cells in the biological sample. In various embodiments, the miRs is isolated from a non-cellular portion of the biological sample. In some embodiments, the miRs is isolated from the total biological sample (e.g., from both intracellular and extracellular portions of the biological sample). In some embodiments, the miRs is assessed or detected by any suitable method. In some embodiments, the miRs is assessed or detected using sequencing.

In some embodiments, the method or assay further includes comparing the expression level to a reference expression level of the one or more miRs. In various cases, an increased or decreased expression level of the one or more miRs relative to the reference expression level indicate that the individual has endometriosis. In some embodiments, the biological sample includes menstrual fluid, cervicovaginal fluid, or both. In some embodiments, the biological sample is disposed in a sample collector as provided herein. In some embodiments, the sample collector comprises a pad, a tampon, a vaginal cup, a cervical cap, a menstrual disk, a cervical disk, a sponge, an interlabial pad, or a combination thereof.

### Treating Endometriosis

Described herein, in certain embodiments, are methods and systems of treating a subject suspected of having endometriosis. In some embodiments, the method comprises obtaining or having obtained a biological sample as described herein from the subject; and performing or having performed an assay on the biological sample to determine if the subject has a biomarker indicative of endometriosis; and if the subject has the biomarker indicative of endometriosis, then administering to the subject an intervention, and if the subject does not have the biomarker indicative of endometriosis, no intervention is administered. In some embodiments, the biomarker indicative of endometriosis is a microRNA expression signature indicative of endometriosis. In some embodiments, the microRNA expression signature indicative of endometriosis comprises a significantly different expression of one or more microRNAs (miRs, miRNAs) selected from the group consisting of: miR-1271-5p, miR-4485-3p, miR-125b-2-3p, miR-410-3p, let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p, and a combination thereof, relative to expression of the one or more microRNAs in an individual not having endometriosis. In some embodiments, the microRNA expression signature indicative of endometriosis comprises a significantly different expression of one or more microRNAs (miRs) selected from the group consisting of: miR-1271-5p, miR-4485-3p, miR-125b-2-3p, miR-410-3p, and a combination thereof, relative to expression of the one or more microRNAs in an individual not having endometriosis.

In some embodiments, the method and systems described herein include performing or having performed the assay on the biological sample to determine if the subject has a biomarker indicative of endometriosis comprises: extracting or having extracted nucleic acid from the biological sample, and sequencing or having sequenced one or more biomarkers from the extracted nucleic acid. In some embodiments, the one or more biomarker is one or more microRNAs. In some embodiments, the biomarkers display differential presence or level in cervicovaginal fluid or menstrual fluid as compared to peripheral blood or cervicovaginal tissue.

In some embodiments, the intervention comprises a surgical intervention, a therapeutic intervention, or a combination thereof. In some embodiments, the surgical intervention comprises surgical removal of at least a part of an endometriosis lesion, hysterectomy, salpingo-oophorectomy, presacral neurectomy, or laparoscopic uterine nerve ablation. In some embodiments, the therapeutic intervention comprises administration of a therapeutic agent. In some embodiments, the therapeutic agent is a hormone, a hormone agonist, hormone antagonist, aromatase inhibitor, an anti-inflammatory therapy, acetyltransferase, histone deacetylase inhibitor, phosphodiesterase inhibitor, or a combination thereof. In some embodiments, the hormone is a synthetic hormone. In some embodiments, the hormone is estrogen, progestin, progesterone, androgen, gonadotropin-releasing hormone (Gn-RH), or a combination thereof. In some embodiments, the hormone agonist is a gonadotropin-releasing hormone (Gn-RH) agonist. In some embodiments, the hormone antagonist is a gonadotropin-releasing hormone (Gn-RH) antagonist. In some embodiments, the therapeutic agent is a birth control comprising the hormone. In some embodiments, the anti-inflammatory therapy is an NSAID, JNK inhibitor, TNF inhibitor, an interleukin (IL) inhibitor, or a combination thereof.

Described herein, in certain embodiments, are methods of predicting the success of a intervention in an individual for endometriosis. In some embodiments, the method comprises taking a biological sample from the individual prior to the intervention, such as a surgery or administration of a therapeutic composition. In some embodiments, the intervention is an intervention to treat endometriosis. In some embodiments, the method comprises performing an assay on the biological sample taking prior to the intervention to determine a biomarker or biomarker signature of the individual. In some embodiments, the method comprises taking a biological sample is taken from the individual after the intervention. In some embodiments, the method comprises performing an assay on the biological sample taking after the intervention to determine a biomarker or biomarker signature of the individual. In some embodiments, the method comprises comparing the biomarker or biomarker signature in the biological sample taken prior to the intervention to the biomarker or biomarker signature in the biological sample taken after the intervention. Predicting the success of the intervention is based off of the biomarker or biomarker signature in the biological sample taken prior to the intervention, the biomarker or biomarker signature in the biological sample taken after to the intervention, a comparison of the biomarker or biomarker signature in the biological sample taken prior to the intervention to the biomarker or biomarker signature in the biological sample taken after to the intervention, or a combination thereof. Predicting the success of the intervention comprises comparing the biomarker or biomarker signature to a known biomarker or biomarker signature from an individual receiving the intervention where the outcome of the individual was known, for example, success of the intervention or failure of the intervention.

### Sample Collection Systems

Described herein, in certain embodiments, are systems or devices for collecting cells and other biological material (nucleic acid, protein, metabolites) from a biological sample, such as menstrual fluid. In some embodiments, the systems or devices are used with the methods described herein. In some embodiments, the system comprises an upper portion; a lower portion; a central portion comprising a first end configured to be operably coupled to the upper portion and a second end operably coupled to the lower portion; and a compression member comprising: a compression first end disposed in the central portion, the compression first end forming a compression base in contact with an inner surface of the central portion, the compression base and the central portion forming a first central cavity configured to receive a sample collector; and a compression second end coupled to the lower portion; wherein the compression member is configured to compress a sample collector upon activation of the lower portion. In some embodiments, the upper portion comprises an upper cavity configured to retain a preservation solution. In some embodiments, the upper cavity is accessible via a disruptable member. In some embodiments, the central portion is configured to retain the preservation solution. In some embodiments, the preservation solution (e.g., disposed within the disruptable member) is disposed at or adjacent the compression base.

In some embodiments, a sample collection device comprises a system for collecting a biological sample from a subject, which comprises a comprising a sample collector that non-invasively collects the biological sample from the subject. In some embodiments, the sample collector is inserted into to subject's vaginal cavity to collect the biological sample. In some embodiments, the system described herein collects a volume of biological sample comprising menstrual fluid, cervicovaginal fluid, secreted mucus, shed uterus cells, shed ovary cells, or other cells, tissue, or fluid. In some embodiments, the sample collector is made of materials that are capable of collecting and/or retaining the biological sample. In some embodiments, the sample collector is made of highly absorbent materials that absorb a liquid sample rapidly. In some embodiments, the sample collector is made of materials that release absorbed liquid samples rapidly, such as when a compression mechanism (e.g., pressure, force) is applied to the sample collector. In some embodiments, the system comprises an extractor for extracting the biological sample from the sample collector. In some embodiments, the extractor comprises a component for applying a compression mechanism to the sample collector. In some embodiments, components for applying compression mechanisms include but are not limited to a spring, threaded screw, lever, air-tight plunger, or roller-based compression. In some embodiments, the liquid sample absorbed on a sample collector is extracted by applying a compression mechanism to the sample collector. In some embodiments, the system comprises the compression mechanism. In some embodiments, the system does not comprise a compression mechanism. In some embodiments, the compression mechanism is compressed outside of the system. In some embodiments, closing or sealing the system activates the compression mechanism. In some embodiments, closing or sealing the system does not activate the compression mechanism. In some embodiments, the compression mechanism is activated separately from closing or sealing the system. In some embodiments, the liquid sample absorbed on a sample collector is extracted without a compression mechanism. In some embodiments, the liquid sample absorbed on a sample collector is eluted into a buffer described herein. In some embodiments, the extractor comprises a sample receptacle that receives the sample collector via an opening, and a reservoir that is in fluid communication with the sample receptacle for receiving the biological sample released from the sample collector. In some embodiments, the reservoir and/or receptacle contains a solution comprising one or more reagents for analyzing, preserving, storing, or transporting the collected biological sample. In some embodiments, the one or more reagents are necessary for hydrolyzing, diffusing, or releasing the biological sample. In some embodiments, the one or more reagents are necessary for analyzing, preserving, or extracting deoxyribonucleic acid, ribonucleic acid, or protein in the biological sample. In some embodiments, the one or more reagents are necessary for reducing analysis background noise. In some embodiments, the one or more reagents are necessary for precipitating or removing a contaminant in the biological sample. In some embodiments, the one or more reagents are necessary for testing the biological sample for a presence or absence of an analyte in the biological sample. In some embodiments, the receptacle contains a reagent that are necessary for dissolving the sample collector upon coming in contact with the sample collector. In some embodiments, accordingly, the sample collector is made of materials that dissolve upon contact with the reagent stored in the receptacle, thereby releasing the biological sample into the reservoir. In some embodiments, the system furthers comprise a cartridge comprising a chamber, wherein the cartridge and/or the chamber is connected to the reservoir via a docking unit, such that upon the cartridge and/or the chamber coming in contact with the reservoir, the released biological sample flows into the cartridge and/or the chamber. In some embodiments, the docking unit comprises a one-way pressure valve. In some embodiments, the docking unit comprises a resealable slit. In some embodiments, the cartridge containing the collected biological sample is covered or sealed. In some embodiments, the cartridge containing the collected biological sample is transported without causing damage or degradation to the collected biological sample.

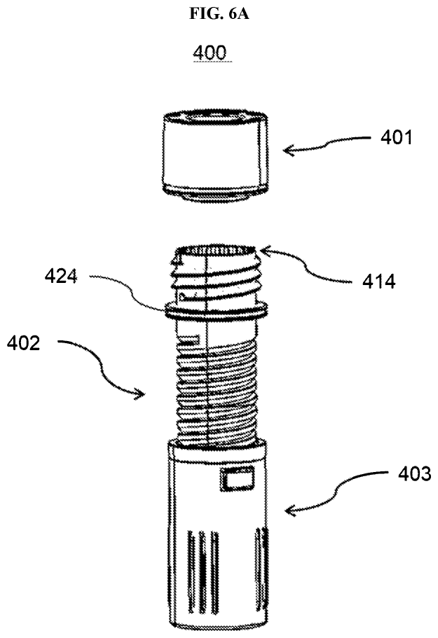

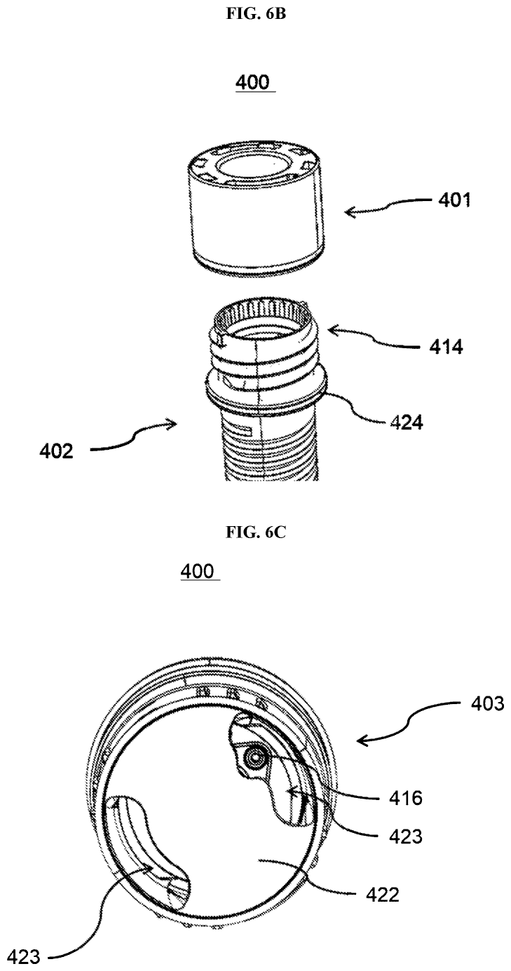

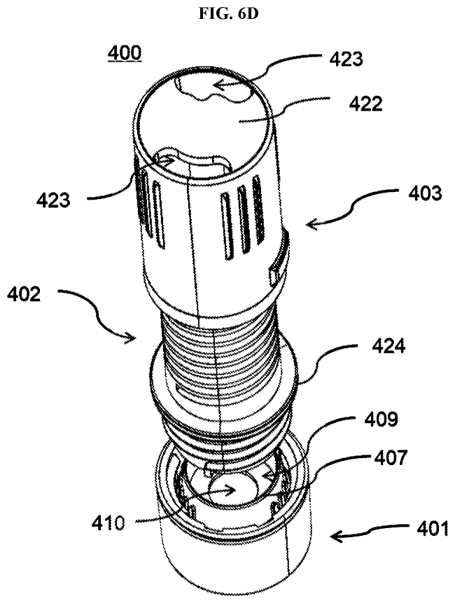

FIG. 5 and FIGS. 6A-6C illustrate an embodiment of the menstrual fluid cell collection system 400 that is used to collect a sample as described herein. In some embodiments, the system 400 comprises an upper portion 401, a central portion 402, a lower portion 403, and a compression member 404.

In some embodiments, the upper portion 401 comprises an upper cavity 405, a disruptable member 406, an inner surface 425 of the upper portion 401, and a disrupting element 407. In some embodiments, the upper portion 401 is coupled to a first end 414 of the central portion 402. In some embodiments, the upper portion 401 is threadably coupleable to the first end 414. In some embodiments, the upper portion 401 is not removably coupleable to the central portion 402, e.g., by a patient. In some embodiments, the upper portion 401 is removably coupleable to the central portion 402 by removal of a screw, or other suitable coupling member, in the upper portion 401, e.g., by a medical practitioner or technician. In some embodiments, the upper portion 401 seals, or is configured to seal, the first central cavity 411. In some embodiments, one of the upper portion 401 or the first end 414 of the central portion 402 comprises a seal such that fluid communication is inhibited from the first central cavity 411 to an exterior of the system (e.g., such that a fluid cannot flow out the first central cavity 411).

In some embodiments, the system 400 further comprises a connector, wherein the connector flexibly couples the upper portion 401 to the central portion 402. In some embodiments, the connector flexibly couples the upper portion 401 to the central portion 402 such that the connector is flexed to allow the upper portion 401 to be coupled to the first end 414 of the central portion 402. In some embodiments, the connector comprises or be formed from polyethylene, polypropylene, polyester, nylon, polyvinyl chloride, polystyrene, poly(methyl methacrylate), polyetheretherketone, rubber, silicone, thermoplastic elastomer (TPE), or a combination thereof.

In some embodiments, the disruptable member 406 encloses the preservation solution (e.g., Biomatrica RNAgard®). In some embodiments, the amount of the preservation solution enclosed by disruptable member 406 ranges from about 3 ml to about 12 ml, about 5 ml to about 10 ml, about 7.5 ml to about 10 ml, or about 7 ml to about 8 ml. In some embodiments, the disruptable member 406 comprises or be formed from polyethylene, polypropylene, polyester, nylon, polyvinyl chloride, polystyrene, poly(methyl methacrylate), polyetheretherketone, aluminum, or a combination thereof. In some embodiments, the aluminum is a heat-sealable aluminum foil.

In some embodiments, the disrupting element 407 comprises a first surface 408, a second surface 409, and an opening 410. In some embodiments, the first surface 408 of the disrupting element 407 is adjacent to the disruptable member 406. In some embodiments, the second surface 409 of the disrupting element 407 is adjacent to a first central cavity 411. In some embodiments, the disrupting element 409 is configured to disrupt the disruptable member 406 upon activation of the upper portion 403. In some embodiments, the disrupting element 409 is displaced toward the disruptable member 406, such that a force is exerted on the disruptable member 406. In some embodiments, the force causes the disruptable member 406 to break, disrupt, fail, or open. In some embodiments, a piercer (e.g., a floating piercer) is disposed within the disruptable member 406 (e.g., within the preservation solution). n some embodiments, upon compression of the disruptable member 406, the piercer breaks, disrupts, or opens the disruptable member 406.

In some embodiments, the disrupting element 409 includes one or more protrusions (e.g., on the first surface 408) that are configured to cut or pierce the disruptable member 406, for example, when the disrupting element 407 is pressed against or displaced toward the disruptable member 406. In some embodiments, the one or more protrusions include a blade, a point, a spike, or another suitable protrusion that is configured to disrupt the disruptable member 406. In some embodiments, coupling the upper portion 401 to a first end 414 of the central portion 402 activate the upper portion 401. In some embodiments, upon activation of the upper portion 401, and/or subsequent disruption of the disruptable member 406, the opening 409 of the disrupting element 410 allows or permit fluid communication between the upper cavity 405 and the first central cavity 411. In some embodiments, stated another way, preservation solution flows out of the disrupted disruptable member 406, flow through the opening 409, and flow into at least a portion of the first central cavity 411. In some embodiments, the preservation solution is configured to flow into or enter the first central cavity 411 upon disruption of the disruptable member.

In some embodiments, the disrupting element 407 is configured to exert a first force on a sample collector upon activation of the lower portion 403, when a sample collector is disposed in the first central cavity 411. In some embodiments, activation of the lower portion 403 (e.g., rotation of the lower portion 403 relative to the central portion 402) displaces or moves the compression first end 418 toward the upper cavity 405. In some embodiments, the activation of the lower portion 403 displaces the compression base 417 toward the upper cavity 405. Displacement of the compression base 417 is configured to exert a second force on a sample collector, e.g., when the sample collector is disposed in the first central cavity 411. In some embodiments, the lower portion 403 provides a mechanical advantage such that a patient is able to compress a sample collector using the system 400. As shown in FIG. 5, in some embodiments, the lower portion 403 is coupled (e.g., threadably coupled) to the central portion 402. In some embodiments, the interaction between the lower portion 403 and the central portion 402 upon activation by a user provides the mechanical advantage such that sufficient force is applied on at least a portion of a sample collector to compress or crush the sample collector. In some embodiments, more than 20 pounds, 30 pounds, 40 pounds, 50 pounds, 60 pounds, 70 pounds, 80 pounds, 90 pounds, or 100 pounds of load is exerted on the sample collector by the system 400. In certain embodiments, less than 200 pounds, 180 pounds, 160 pounds, 140 pounds, 120 pounds, 100 pounds, or 80 pounds of load is exerted on the sample collector by the system 400.

In some embodiments, the central portion 402 comprises a first central cavity 411, a second central cavity 412, an inner surface 413, a first end 414, and a second end 415. In some embodiments, the central portion 402 further comprises a stopper 424. In some embodiments, the central portion 402 is coupled to the lower portion 403. In some embodiments, the central portion 402 is threadably coupled to the lower portion 403. In some embodiments, the lower portion 403 is rotatable in a first direction relative to the central portion 402. In some embodiments, the first direction relative to the central portion 402 is a clockwise rotation. In some embodiments, the lower portion 403 is not rotatable in a second direction relative to the central portion 402. In some embodiments, the second direction relative to the central portion 402 is a counterclockwise rotation. In some embodiments, the first central cavity 411 is disposed between the compression base 417 and the first end of the central portion 414. In some embodiments, the compression base 417 and the central portion 402 further forms the second central cavity 412. In some embodiments, the second central cavity 412 is configured to receive the preservation solution and a biological sample from a sample collector.

In some embodiments, the central portion 402 comprises a port 416. In some embodiments, the port 416 is disposed through at least a portion of the second end 415 of the central portion 415. In some embodiments, the port 416 permits access to the second central cavity 412. In some embodiments, the port 416 is a valve. In some embodiments, the valve is a self-sealing valve, a relief valve, a sampling valve, a one-way valve, a check valve, a duckbill valve, a flapper valve, an umbrella valve, a septum, or other suitable valve. In some embodiments, the port 416 is accessed via a syringe (e.g., a syringe is displaceable through at least a portion of the port 416). In some embodiments, the central portion 402 comprises one, two, three, four, five, or more than five ports. In some embodiments, the port 416 is accessed through an external opening 423 on the base 422.

In some embodiments, the compression member 404 comprises a compression base 417, a compression first end 418, and a compression second end 419. In some embodiments, the compression base 417 comprises a compression base seal 420 and an outer surface 421 of the compression base 417. In some embodiments, the compression base seal 420 comprises or be formed from a nitrile, ethylene-propylene rubber, perfluoroelastomer (FFKM), fluorosilicone, neoprene, chloroprene, polyurethane, silicone, fluorocarbon, or a combination thereof. In some embodiments, the ethylene-propylene rubber is an ethylene-propylene co-polymer (EPR) or an ethylene-propylene-diene terpolymer (EPDM).

In some embodiments, a portion (e.g., an elongate member) of the compression member extend through at least a portion of the second end 415 of the central portion 402. In some embodiments, the compression base 417 comprises the compression base seal 420 such that fluid communication is permitted in a first direction around at least a portion of the compression base 417 and inhibited or limited in a second direction around at least a portion of the compression base 417. In some embodiments, the first direction is from the first central cavity 411 to the second central cavity 412. In some embodiments, the second direction is from the second central cavity 412 to the first central cavity 411. In some embodiments, the compression base seal 420 extends around an outer surface 421 of the compression base 417. In some embodiments, the compression base seal 420 is disposed between the compression base 417 and the inner surface 413 of the central portion 402. In some embodiments, when a portion of the compression member 404 extends through an aperture or opening in the second end 415 of the central portion 402, the aperture comprises a seal such that fluid communication is inhibited between the second central cavity and an exterior of the central portion.

In some embodiments, the first central cavity 411 at a position adjacent the first end 414 of the central portion 402 has a first diameter. In some embodiments, the second central cavity 412 at a position adjacent the second end 415 of the central portion 402 has a second diameter. In some embodiments, the first and second central cavities 411, 412 at one or more positions between the first end 414 and the second end 415 has a third diameter. In some embodiments, the diameter of the first and second central cavities 411, 412 at one or more positions between the first end 414 and the second end 415 increases or decreases. In some embodiments, the diameter gradually increases or decreases (e.g., the inner surface 413 is sloped).

In certain embodiments, the first diameter and the second diameter is substantially equal. In various embodiments, the first and second diameters is less or smaller than the third diameter. In some embodiments, when the compression base 417 is disposed adjacent the second end 415 of the central portion 402, the compression base seal 420 forms a seal between the compression base 417 and the inner surface 413 of the central portion 402. In some embodiments, when the compression base 417 is disposed adjacent the first end 414 of the central portion 402, the compression base seal 420 forms a seal between the compression base 417 and the inner surface 413 of the central portion 402. In some embodiments, when the compression base 417 is disposed at a position between the first end 414 and the second end 415 of the central portion 402, the compression base seal 420 does not form a seal between the compression base 417 and the inner surface 413 of the central portion 402. In some embodiments, upon displacement of the compression base 417 between the first end 414 and the second end 415 of the central portion 402, a seal is not formed between the compression base 417 and the inner surface 413 of the central portion 402. In some instances, if a seal was formed at one or more positions between the first and second ends 414, 415 of the central portion 402, too much pressure builds up in the system 400 (e.g., within one or more of the first or second central cavities 411, 412). In some embodiments, the compression base seal 420 is a wiper seal. In some embodiments, the wiper seal relieves or is configured to relieve pressure in the system 400 (e.g., by allowing passage of fluid or air) if pressure increases above a threshold level within at least a portion of the system 400.

With reference to FIG. 5, in some embodiments, the system 400 further comprises one or more ridges or ribs 427. As illustrated, the ridge 427 extends along a portion of the inner surface 413 of the central portion 402. In some embodiments, one or more recesses extend along a portion of the inner surface 413 of the central portion 402. In some embodiments, the ridge 427 or recess interrupts the formation of a seal between the compression base 417 and the inner surface 413 of the central portion 402 at one or more positions between the first end 414 and the second end 415 of the central portion 402 such that pressure does not increase above a threshold level within at least a portion of the system 400 (e.g., the development of too much pressure is avoided or inhibited within the system 400).

In some embodiments, the lower portion 403 comprises a base 422. In some embodiments, the base 422 comprises an external opening 423. In some embodiments, the base 422 comprises one, two, three, four, five, or more than five openings. In some embodiments, the external opening 423 allows, permits, or provides access to the port 416.

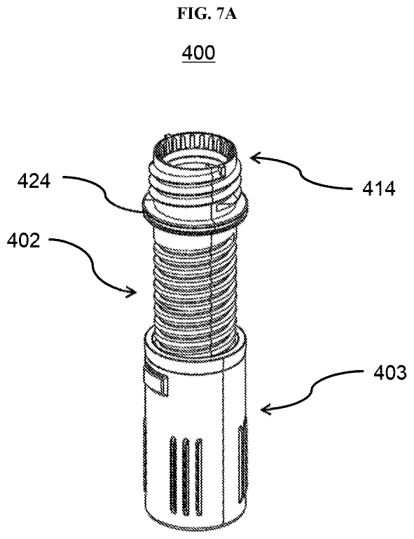

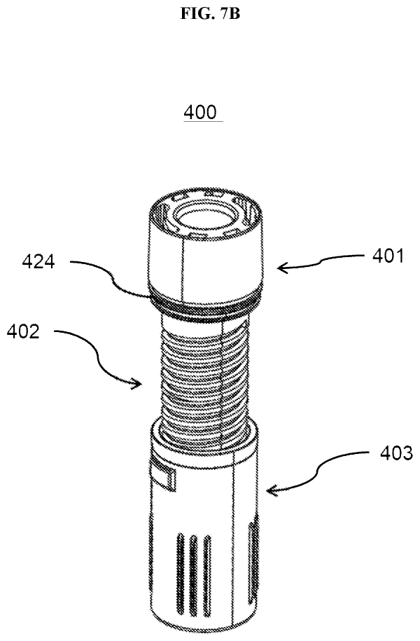

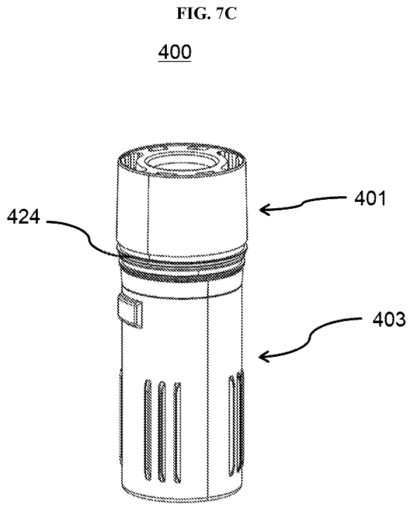

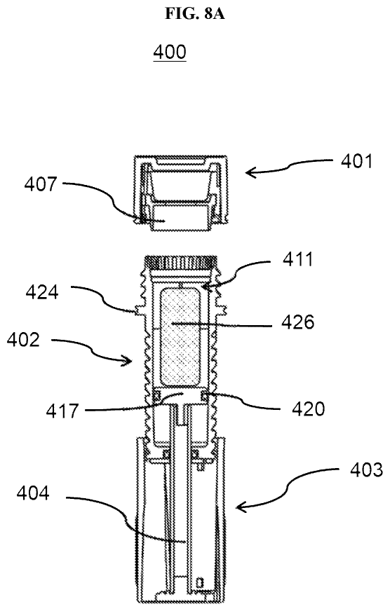

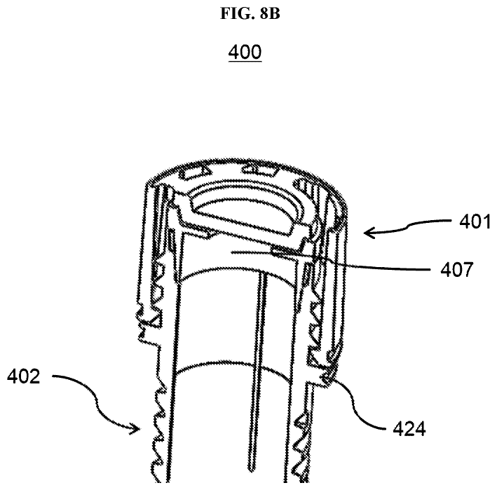

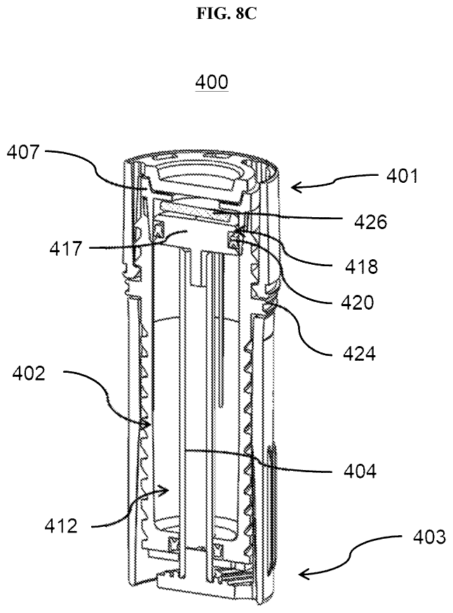

FIGS. 7A-7C and 8A-8C illustrate use of an embodiment of the system 400 described herein. In some embodiments, the upper portion 401, while configured to be operably connected to the central portion 402, is not be connected to the central portion 402 prior to insertion of the sample collector via the first end of the central portion 414 (FIG. 7A). In some embodiments, the lower portion 403 is operably coupled to the central portion 402 prior to insertion of a sample collector via the first end 414 of the central portion 402 (FIG. 7A).

In some embodiments, a sample collector 426 is placed into a first central cavity 411 of the system 400 (FIG. 8A). In some embodiments, following insertion of the sample collector 426 into the first central cavity 411 via the first end 414 of the central portion 402, the upper portion 401 is operably coupled to the central portion 402 (FIG. 7B). Operably coupling the upper portion 401 to the central portion 402 comprises threadably coupling the upper portion 401 to the central portion 402 and rotating the upper portion 401 in a first direction to activate the upper portion 401. In some embodiments, the upper portion 401 is rotated until the upper portion 401 connects with or contacts the stopper 424 (FIG. 7B).

In some embodiments, the upper portion 401 is rotated until a signal (e.g., a haptic signal) is given by the system. In some embodiments, the signal is a sound. In some embodiments, the sound is a click. Activation of the upper portion 401 by rotation of the upper portion 401 comprises decreasing the distance between the disrupting element 407 and the inner surface of the upper portion 425 (FIG. 8B). In some embodiments, activation of the upper portion 401 comprises rotation of the upper portion 401 in a first direction relative to the central portion 402. In some embodiments, the first direction is clockwise. In some embodiments, the upper portion 401 is not be rotatable in a second direction relative to the central portion 402. In some embodiments, the second direction is counterclockwise. In some embodiments, the space between the disrupting element 407 and the inner surface of the upper portion 425 comprises the upper cavity 405 which houses the disruptable member 406.

In some embodiments, activation of the upper portion 401 is completed when the upper portion 401 connects with or contacts the stopper 424 or when the signal is given by the system. In some embodiments, activation of the upper portion 401 is completed when no additional rotation of the upper portion 401 in the first direction is achieved or performed. In some embodiments, activation of the upper portion 401 is completed when the disruptable member 406 ruptures. In some embodiments, activation of the upper portion 401 results in compression of the sample collector 426 (FIG. 8C).

In some embodiments, following activation of the upper portion 401, the lower portion 403 is activated. In some embodiments, activation of the lower portion 403 comprises rotation of the lower portion 403 in a first direction relative to the central portion 402. In some embodiments, the lower portion 403 is rotated until the lower portion 403 connects with or contacts the stopper 424 (FIG. 7C). In some embodiments, the lower portion 403 is rotated until a signal (e.g., a haptic signal) is given by the system. In some embodiments, the signal is a sound. In some embodiments, the sound is a click. In some embodiments, activation of the lower portion 403 is completed when no additional rotation of the lower portion 403 in the first direction is achieved or performed. In some embodiments, activation of the lower portion 403 displaces the compression first end 418 of the compression base 417 toward the disrupting element 407 (FIG. 8B and FIG. 8C). In some embodiments, the disrupting element 407 is configured to exert a first force on a sample collector upon activation of the lower portion 403. In some embodiments, the first force compresses the sample collector between the compression first end 418 and the disrupting element 407. In some embodiments, compression of the sample collector results in the preservation solution being mixed with the biological sample entering into the second central cavity 412. In various embodiments, the upper portion 401 is coupled to the central portion 402. In such a configuration the upper portion 401 is sealed to the central portion 402. In some embodiments, formation of the seal is indicated by the haptic signal (e.g., the click),In some embodiments, following coupling of the upper portion 401 to the central portion 402 and formation of the seal, the lower portion 403 is activated such that the preservation solution is released from the disruptable member 406. A In some embodiments, the preservation solution does not leak out or flow out of the system 400 (e.g., around the seal) and contact the user.

In some embodiments, prior to completion of the activation of the lower portion 403, the compression base seal 420 allows fluid communication between the first central cavity 411 and the second central cavity 412. In some embodiments, fluid communication between the first central cavity 411 and the second central cavity 412 allows the preservation solution mixed with the biological sample to enter into the second central cavity 412. In some embodiments, completion of the activation of the lower portion 403 inhibits or prevents fluid communication by or around the compression base seal 420 between the first central cavity 411 and the second central cavity 412. In some embodiments, activation of the lower portion 403 is completed when the lower portion 403 connects with or contacts the stopper 424, when a signal is given by the system, when no additional rotation of the lower portion 403 in the first direction is achieved or performed, or a combination thereof.

Described herein, in certain embodiments, are kits comprising a system as described herein and a sample collector (e.g., a tampon). In some embodiments, the kit includes an identifying description, a label, and/or a package insert. In some embodiments, the kit further comprise a shipping packet. In some embodiments, the shipping packet is used for shipment of the system after use. In some embodiments, the shipping packet comprises a hydrophilic material. In some embodiments, the hydrophilic material comprises cotton, cellulose, hydrogel, absorbent polymers, or a combination thereof. In some embodiments, the shipping packet comprises 1, 2, 3, 4, 5, or more than 5 layers of the hydrophilic material. If the shipping packet comprises more than two layers of hydrophilic material, at least one layer of the more than two layers of hydrophilic material is different from the remaining layers. In some embodiments, the hydrophilic material is contained in a pouch. In some embodiments, the pouch is formed from polyethylene, polypropylene, polyester, nylon, polyvinyl chloride, polystyrene, poly(methyl methacrylate), polyetheretherketone, or a combination thereof. In some embodiments, the shipping packet comprises an adhesive strip, a glue, or a waterproof zipper for sealing of the shipping packet after the system comprising the sample collector has been placed inside. In some embodiments, the shipping packet is pre-labeled. In some embodiments, the shipping packet further comprises at least one layer of absorbent material.

In some embodiments, the kit further comprises a label or a package insert. In some embodiments, the label or package insert comprises a list of the contents of the kit, instructions relating to the kit's use in the methods described herein, or a combination thereof. In some embodiments, the label is on or associated with the system. In some embodiments, the label is on a system when letters, numbers, or other characters forming the label are attached, molded, or etched into the system itself. In some embodiments, the label is associated with a system when it is present within a receptacle or carrier that also holds the system, e.g., as a package insert. In some instances, the label is used to indicate that the contents are to be used for a specific application, such as collection of a sample from a menstrual fluid.

### Numbered Embodiments

The disclosure herein is further defined by the following numbered embodiments. 1. An assay for classifying or detecting endometriosis in a subject comprising determining from a menstrual fluid sample of the subject an expression level of one or more microRNAs (miRs), wherein the menstrual fluid sample is collected on a first, second, third, fourth, fifth, sixth, and/or seventh day of the subject's menstrual cycle. 2. The assay of embodiment 0, wherein the menstrual fluid sample is collected on the second day of the subject's menstrual cycle. 3. The assay of embodiment 0 or embodiment [0163], further comprising determining from the menstrual fluid sample of the subject an expression level of two or more microRNAs (miRs). 4. The assay of embodiment 0 or embodiment [0163], further comprising determining from the menstrual fluid sample of the subject an expression level of three or more microRNAs (miRs). 5. The assay of any one of embodiments 1-[0163], wherein the miRs are selected from the group consisting of: miR-1271-5p, miR-4485-3p, miR-125b-2-3p, and miR-410-3p. 6. The assay of any one of embodiments 1-[0163], further comprising comparing the expression level to a reference expression level of the one or more miRs, wherein an increased or decreased expression level of the one or more miRs relative to the reference expression level indicates that the subject has endometriosis. 7. The assay of any one of embodiments 1-[0163], wherein the miRs are intracellular miRs. 8. The assay of any one of embodiments 1-[0163], wherein the miRs are extracellular miRs. 9. The assay of any one of embodiments 1-[0163], wherein the miRs are intracellular and extracellular miRs. 10. The assay of any one of embodiments 1-[0163], wherein the menstrual fluid further comprises cervicovaginal fluid. 11. The assay of any one of embodiments [0163]-[0163], wherein the menstrual fluid sample is disposed in a sample collector. 12. The assay of embodiment [0163], wherein the sample collector is a pad, a tampon, a vaginal cup, a cervical cap, a menstrual disk, a cervical disk, a sponge, or an interlabial pad. 13. A system for collecting a biological sample from a sample collector, the system comprising: an upper portion; a lower portion; a central portion comprising a first end configured to be operably coupled to the upper portion and a second end operably coupled to the lower portion; a disruptable member retaining a preservation solution; and a compression member comprising: a compression first end disposed in the central portion, the compression first end forming a compression base in contact with an inner surface of the central portion, the compression base and the central portion forming a first central cavity configured to receive a sample collector; and a compression second end coupled to the lower portion; wherein the compression member is configured to compress a sample collector upon activation of the lower portion such that the disruptable member is disrupted and releases the preservation solution. 14. The system of embodiment 13, wherein the disruptable member is disposed in an upper cavity of the upper portion. 15. The system of embodiment 13 or embodiment [0163], further comprising a disrupting element configured to disrupt the disruptable member upon activation of the upper portion. 16. The system of embodiment [0163], wherein the disrupting element comprises an opening such that fluid communication is permitted between the upper cavity and the first central cavity. 17. The system of embodiment [0163] or embodiment [0163], wherein the disrupting element comprises a first surface adjacent the disruptable member and a second surface adjacent the first central cavity. 18. The system of any one of embodiments [0163]—[0163], wherein the disrupting element is configured to exert a first force on a sample collector upon activation of the lower portion, when a sample collector is disposed in the first central cavity. 19. The system of any one of embodiments [0163]-[0163], wherein activation of the lower portion displaces the disrupting element toward the upper cavity, when a sample collector is disposed in the first central cavity. 20. The system of any one of embodiments 1-[0163], wherein the disruptable member encloses the preservation solution. 21. The system of any one of embodiments 1-[0163], wherein the disruptable member comprises polyethylene, polypropylene, polyester, nylon, polyvinyl chloride, polystyrene, poly(methyl methacrylate), polyetheretherketone, aluminum foil, or a combination thereof. 22. The system of embodiment [0163], wherein the aluminum foil is heat sealable. 23. The system of any one of embodiments 1-[0163], wherein the preservation solution is configured to flow into the first central cavity upon disruption of the disruptable member. 24. The system of embodiment 13, wherein the disruptable member is disposed adjacent the compression base. 25. The system of embodiment [0163], wherein the disruptable member encloses the preservation solution. 26. The system of embodiment [0163] or embodiment [0163], wherein the disruptable member comprises polyethylene, polypropylene, polyester, nylon, polyvinyl chloride, polystyrene, poly(methyl methacrylate), polyetheretherketone, aluminum foil, or a combination thereof. 27. The system of embodiment [0163], wherein the aluminum foil is heat sealable. 28. The system of any one of embodiments [0163]-[0163], wherein the preservation solution is configured to flow into the first central cavity upon disruption of the disruptable member. 29. The system of any one of embodiments 13-[0163], wherein the upper portion is configured to seal the central cavity. 30. The system of any one of embodiments 13-[0163], wherein one of the upper portion or the first end of the central portion comprises a seal such that fluid communication is inhibited from the first central cavity to an exterior of the system. 31. The system of any one of embodiments [0163]-[0163], wherein the upper portion is threadably coupleable to a first end of the central portion. 32. The system of embodiment [0163], wherein the upper portion is not removably coupleable to the central portion by a patient. 33. The system of embodiment [0163] or embodiment [0163], wherein the upper portion is rotatable in a first direction relative to the central portion by a patient, and wherein the lower portion is not rotatable in a second direction relative to the central portion by the patient. 34. The system of any one of embodiments [0163]-[0163], wherein the first cavity is disposed between the compression base and the first end of the central portion. 35. The system of any one of embodiments [0163]-[0163], wherein the compression base and the central portion further form a second central cavity configured to receive the preservation solution and a biological sample from a sample collector. 36. The system of any one of embodiments [0163]-[0163], wherein activation of the lower portion displaces the compression base toward the upper cavity. 37. The system of embodiment [0163], wherein the displacement of the compression base is configured to exert a second force on a sample collector. 38. The system of any one of embodiments [0163]-[0163], wherein the central portion is threadably coupled to the lower portion. 39. The system of embodiment [0163], wherein the threadable coupling of the central portion to the lower portion provides a mechanical advantage. 40. The system of embodiment [0163] or embodiment [0163], wherein the lower portion is rotatable in a first direction relative to the central portion by a patient, and wherein the lower portion is not rotatable in a second direction relative to the central portion by the patient. 41. The system of any one of embodiments 1-[0163], wherein a portion of the compression member extends through the second end of the central portion. 42. The system of embodiment 41, wherein the portion of the compression member extends through an aperture in the second end of the central portion, and wherein the aperture comprises a seal such that fluid communication is inhibited between the second central cavity and an exterior of the central portion. 43. The system of any one of embodiments 1-42, wherein the compression base comprises a compression base seal such that fluid communication is permitted in a first direction and inhibited in a second direction. 44. The system of embodiment 43, wherein the compression base seal extends around an outer surface of the compression base. 45. The system of embodiment 43 or embodiment 44, wherein the compression base seal is disposed between the compression base and the inner surface of the central portion. 46. The system of any one of embodiments 43-45, wherein the first direction is from the first central cavity to the second central cavity, and wherein the second direction is from the second central cavity to the first central cavity. 47. The system of any one of embodiments 1-46, wherein the central portion comprises a port. 48. The system of embodiment 47, wherein the port is disposed through the second end of the central portion. 49. The system of embodiment 47 or embodiment 48, wherein the port permits access to the second central cavity. 50. The system of any one of embodiments 47-49, wherein the port is a valve. 51. The system of embodiment 50, wherein the valve is a self-sealing valve, a septum, a check valve, a relief valve, or a sampling valve. 52. The system of any one of embodiments 1-50, wherein the sample collector is a pad, a tampon, a vaginal cup, a cervical cap, a menstrual disk, a cervical disk, a sponge, or an interlabial pad. 53. The system of any one of embodiments 1-52, wherein the volume of preservation solution is from 5 ml to 10 ml. 54. The system of embodiment 53, wherein the volume of the preservation solution is about 7.5 mL. 55. The system of any one of embodiments 1-54, wherein the osmolality of the preservation solution is from about 310 to about 410 mOsm kg-1. 56. The system of any one of embodiments 1-54, wherein the osmolality of the preservation solution is from about 95 to about 210 mOsm kg-1. 57. A kit comprising: a system of any one of embodiments 1-56; and a sample collector. 58. The kit of embodiment 57, wherein the sample collector is a pad, a tampon, a vaginal cup, a cervical cap, a menstrual disk, a cervical disk, a sponge, or an interlabial pad. 59. The kit of embodiment 57 or embodiment 58, further comprising a shipping packet. 60. The kit of embodiment 59, wherein the shipping packet comprises a hydrophilic material. 61. The kit of embodiment 60, wherein the hydrophilic material comprises cotton, cellulose, hydrogel, absorbent polymers, or a combination thereof. 62. The kit of embodiment 60 or embodiment 61, wherein the shipping packet comprises at least one layer of the hydrophilic material. 63. The kit of embodiment 60 or embodiment 61, wherein the hydrophilic material is contained in a pouch. 64. The kit of embodiment 63, wherein the pouch is formed from polyethylene, polypropylene, polyester, nylon, polyvinyl chloride, polystyrene, poly(methyl methacrylate), polyetheretherketone, or a combination thereof. 65. The kit of embodiment 59, wherein the shipping packet comprises a means for sealing the shipping packet. 66. The kit of embodiment 65, wherein the means for sealing the shipping packet comprises an adhesive strip, a glue, a waterproof zipper, or a combination thereof. 67. The kit of any one of embodiments 59-66, wherein the shipping packet comprises a label. 68. The kit of any one of embodiments 57-67, further comprising instructions for use of the system. 69. A biological sample collected using the system of any one of embodiments 1-56 or the kit of any one of embodiments 57-68. 70. A method of collecting a biological sample from a sample collector, the method comprising: a. obtaining a device comprising: an upper portion comprising an upper cavity configured to retain a preservation solution, wherein the upper cavity is accessible via a disruptable member; a lower portion; a central portion comprising a first end configured to be operably coupled to the upper portion and a second end operably coupled to the lower portion; and a compression member comprising: a first compression end disposed in the central portion, the first compression end forming a compression base in contact with an inner surface of the central portion, the compression base and the central portion forming a first central cavity configured to receive a sample collector; and a second compression end coupled to the lower portion; wherein the compression member is configured to compress a sample collector upon activation of the lower portion; and b. placing a sample collector into the first central cavity; c. activating the lower portion to compress the sample collector to release the biological sample from the sample collector; and d. collecting the biological sample. 71. The method of embodiment 70, wherein the sample collector is a pad, a tampon, a vaginal cup, a cervical cap, a menstrual disk, a cervical disk, a sponge, or an interlabial pad. 72. The method of embodiment 70 or embodiment 71, wherein activating the lower portion comprises rotating the lower portion in a first direction relative to the central portion. 73. The method of any one of embodiments 70-72, wherein the compression base and the central portion further form a second central cavity configured to receive the preservation solution and a biological sample from a sample collector. 74. The method of any one of embodiments 70-73, wherein the collecting comprises extracting the biological sample from the second central cavity. 75. The method of embodiment 74, wherein the extracting the biological sample is through a port permitting access to the second central cavity on the device. 76. The method of embodiment 75, wherein the port is a valve. 77. The method of embodiment 76, wherein the valve is a self-sealing valve, a septum, a check valve, a relief valve, or a sampling valve. 78. The method of any one of embodiments 75-77, wherein the port is disposed through the second end of the central portion. 79. The method of embodiment 78, wherein the collecting comprises extracting the biological sample from the second central cavity through a syringe inserted into the second central cavity through the port. 80. A method of detecting endometriosis in an individual, comprising determining from a biological sample of the individual an expression level of one or more microRNAs (miRs) selected from the group consisting of: miR-1271-5p, miR-4485-3p, miR-125b-2-3p, and miR-410-3p. 81. The method of embodiment 80, further comprising comparing the expression level to a reference expression level of the one or more miRs, wherein an increased or decreased expression level of the one or more miRs relative to the reference expression level indicates that the subject has endometriosis. 82. The method of embodiment 80, wherein the reference expression level is obtained from a subject not suffering from a reproductive disorder or not suspected of having the reproductive disorder. 83. The method of embodiment 82, wherein the reproductive disorder is endometriosis. 84. The method of any one of embodiments 80-83, wherein the individual suffers from chronic pelvic pain, infertility, heavy menstrual bleeding, or a combination thereof. 85. The method of any one of embodiments 80-84, wherein the endometriosis is deep infiltrating endometriosis (DIE), superficial peritoneal endometriosis (SPE), or ovarian endometriomas (OE). 86. The method of any one of embodiments 80-85, further comprising determining from the biological sample of the individual an expression level of one more miRs selected from the group consisting of: let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p. 87. The method of any one of embodiments 80-86, further comprising determining a methylation profile of one or more CpG sites selected from the CpG sites in Table 4. 88. The method of any one of embodiments 80-87, further comprising determining a measure of bacterial diversity in the biological sample. 89. The method of embodiment 88, wherein the measure of bacterial diversity is an amount of at least one bacterium. 90. The method of embodiment 89, wherein the at least one bacterium is a bacterium in a genus selected from the group consisting of: Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella. 91. The method of any one of embodiments 88-90, further comprising comparing the measure of bacterial diversity to a reference measure of bacterial diversity. 92. The method of any one of embodiments 88-91, wherein the measure of bacterial diversity is a ratio of at least one first bacterium to at least one second bacterium. 93. The method of any one of embodiments 80-92, further comprising determining an amount of Propionibacterium acnes. 94. The method of embodiment 93, further comprising comparing the amount of Propionibacterium acnes to a reference amount of Propionibacterium acnes. 95. The method of any one of embodiments 80-94, wherein the biological sample is a menstrual fluid. 96. The method of embodiment 95, wherein the menstrual fluid further comprises cervicovaginal fluid. 97. The method of 95 or embodiment 96, wherein the biological sample is collected on a second day of the individual's menstrual cycle. 98. The method of any one of embodiments 95-97, wherein the biological sample is collected on a day of the individual's menstrual cycle where the individual experiences a heavy flow of menstrual fluid. 99. The method of any one of embodiments 80-98, wherein the biological sample is collected prior to administering a treatment to the individual. 100. The method of any one of embodiments 80-98, wherein the biological sample is collected after administering a treatment to the individual. 101. The method of any one of embodiments 80-100, wherein the biological sample is disposed in a sample collector. 102. The method of embodiment 101, wherein the sample collector is a pad, a tampon, a vaginal cup, a cervical cap, a menstrual disk, a cervical disk, a sponge, or an interlabial pad. 103. The method of any one of embodiments 80-102, further comprising administering a treatment to the individual for endometriosis. 104. The method of embodiment 103, wherein the treatment is selected from the group consisting of a surgical intervention, administration of therapeutic agent, and a combination thereof. 105. The method of embodiment 104, wherein the therapeutic agent selected from the group consisting of: a hormone, a hormone agonist, a hormone antagonist, an aromatase inhibitor, an anti-inflammatory therapy, an acetyltransferase, a histone deacetylase inhibitor, a phosphodiesterase inhibitor, and a combination thereof. 106. The method of any one of embodiments 80-105, further comprising generating a report based on the expression level of the one or more miRs relative to the reference expression level. 107. The method of embodiment 106, further comprising transmitting the report to a health practitioner. 108. The method of embodiment 106 or embodiment 107, wherein the report contains a recommendation for administering a therapeutic agent to the individual. 109. The method of any one of embodiments 106-108, wherein the report contains a recommendation for surgical intervention. 110. The method of any one of embodiments 80-109, wherein the method has a false discovery rate of 5% or less. 111. A method of detecting endometriosis in an individual comprising determining from a biological sample of the individual, a measurement from the group consisting of: a. a methylation profile of one or more CpG sites selected from the CpG sites in Table 4; b. a measure of bacterial diversity in the biological sample; and c. a combination thereof. 112. The method of embodiment 111, wherein the measure of bacterial diversity is an amount of at least one bacterium. 113. The method of embodiment 112, wherein the at least one bacterium is a bacterium in a genus selected from the group consisting of: Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella. 114. The method of embodiment 112 or embodiment 113, further comprising comparing the measure of bacterial diversity to a reference measure of bacterial diversity. 115. The method of any one of embodiments 112-114, wherein the measure of bacterial diversity is a ratio of at least one first bacterium to at least one second bacterium. 116. The method of any one of embodiments 111-115, further comprising determining an amount of Propionibacterium acnes. 117. The method of embodiment 116, further comprising comparing the amount of Propionibacterium acnes to a reference amount of Propionibacterium acnes. 118. The method of any one of embodiments 111-117, wherein the biological sample is a menstrual fluid. 119. The method of embodiment 118, wherein the menstrual fluid further comprises cervicovaginal fluid. 120. The method of 118 or embodiment 119, wherein the biological sample is collected on a second day of the individual's menstrual cycle. 121. The method of any one of embodiments 118-120, wherein the biological sample is collected on a day of the individual's menstrual cycle where the individual experiences a heavy flow of menstrual fluid. 122. The method of any one of embodiments 111-121, wherein the biological sample is collected prior to administering a treatment to the individual. 123. The method of any one of embodiments 111-121, wherein the biological sample is collected after administering a treatment to the individual. 124. The method of any one of embodiments 111-123, wherein the biological sample is disposed in a sample collector. 125. The method of embodiment 124, wherein the sample collector is a pad, a tampon, a vaginal cup, a cervical cap, a menstrual disk, a cervical disk, a sponge, or an interlabial pad. 126. The method of any one of embodiments 111-125, further comprising administering a treatment to the individual for endometriosis. 127. The method of embodiment 126, wherein the treatment is selected from the group consisting of a surgical intervention, administration of therapeutic agent, and a combination thereof. 128. The method of embodiment 127, wherein the therapeutic agent selected from the group consisting of: a hormone, a hormone agonist, a hormone antagonist, an aromatase inhibitor, an anti-inflammatory therapy, an acetyltransferase, a histone deacetylase inhibitor, a phosphodiesterase inhibitor, and a combination thereof. 129. The method of any one of embodiments 111-128, further comprising generating a report based on the expression level of the one or more miRs relative to the reference expression level. 130. The method of embodiment 129, further comprising transmitting the report to a health practitioner. 131. The method of embodiment 129 or embodiment 107, wherein the report contains a recommendation for administering a therapeutic agent. 132. The method of any one of embodiments 129-131, wherein the report contains a recommendation for surgical intervention. 133. The method of any one of embodiments 111-132, wherein the method has a false discovery rate of 5% or less. 134. A method of detecting endometriosis in an individual, comprising: a. determining from a biological sample of the individual an expression level of one or more microRNAs selected from the group consisting of: miR-1271-5p, miR-4485-3p, miR-125b-2-3p, and miR-410-3p; and b. applying a classifier algorithm to the expression level of the one or more microRNAs, thereby generating a classification of the individual. 135. The method of embodiment 134 wherein the individual suffers from chronic pelvic pain, infertility, heavy menstrual bleeding, or a combination thereof. 136. The method of embodiment 134 or embodiment 135, wherein the endometriosis is deep infiltrating endometriosis (DIE), superficial peritoneal endometriosis (SPE), or ovarian endometriomas (OE). 137. The method of any one of embodiments 134-136, further comprising determining from the biological sample of the individual an expression level of one more miRs selected from the group consisting of: let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p. 138. The method of any one of embodiments 134-137, further comprising determining a methylation profile of one or more CpG sites selected from the CpG sites in Table 4. 139. The method of embodiment 138, further comprising applying the classifier algorithm to the methylation profile. 140. The method of any one of embodiments 134-139, further comprising determining a measure of bacterial diversity in the biological sample. 141. The method of embodiment 140, wherein the measure of bacterial diversity is an amount of at least one bacterium. 142. The method of embodiment 141, wherein the at least one bacterium is a bacterium in a genus selected from the group consisting of: Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella. 143. The method of any one of embodiments 140-142, wherein the measure of bacterial diversity is a ratio of at least one first bacterium to at least one second bacterium. 144. The method of any one of embodiments 140-143, further comprising determining an amount of Propionibacterium acnes. 145. The method of any one of embodiments 140-144, further comprising applying the classifier algorithm to the measure of bacterial diversity. 146. The method of any one of embodiment 134-145, wherein the classification is selected from the group consisting of: likely endometriosis and not likely endometriosis. 147. The method of embodiment 146, wherein the classification of likely endometriosis is selected from the group consisting of: high likelihood of endometriosis, moderate likelihood of endometriosis, and low likelihood of endometriosis. 148. The method of any one of embodiment 134-147, wherein the classifier algorithm comprises a decision tree, random forest, Bayesian network, support vector machine, neural network, or logistic regression algorithm. 149. The method of any one of embodiments 134-148, wherein the biological sample is a menstrual fluid. 150. The method of embodiment 149, wherein the menstrual fluid further comprises cervicovaginal fluid. 151. The method of 149 or embodiment 150, wherein the biological sample is collected on a second day of the individual's menstrual cycle. 152. The method of any one of embodiments 149-151, wherein the biological sample is collected on a day of the individual's menstrual cycle where the individual experiences a heavy flow of menstrual fluid. 153. The method of any one of embodiments 134-152, wherein the biological sample is collected prior to administering a treatment to the individual. 154. The method of any one of embodiments 134-152, wherein the biological sample is collected after administering a treatment to the individual. 155. The method of any one of embodiments 134-154, wherein the biological sample is disposed in a sample collector. 156. The method of embodiment 155, wherein the sample collector is a pad, a tampon, a vaginal cup, a cervical cap, a menstrual disk, a cervical disk, a sponge, or an interlabial pad. 157. The method of any one of embodiments 134-156, further comprising administering a treatment to the individual for endometriosis. 158. The method of embodiment 157, wherein the treatment is selected from the group consisting of a surgical intervention, administration of therapeutic agent, and a combination thereof. 159. The method of embodiment 158, wherein the therapeutic agent selected from the group consisting of: a hormone, a hormone agonist, a hormone antagonist, an aromatase inhibitor, an anti-inflammatory therapy, an acetyltransferase, a histone deacetylase inhibitor, a phosphodiesterase inhibitor, and a combination thereof. 160. The method of any one of embodiments 134-159, further comprising generating a report based on the disease state. 161. The method of embodiment 160, further comprising transmitting the report to a health practitioner. 162. The method of embodiment 160 or embodiment 161, wherein the report contains a recommendation for administering a therapeutic agent. 163. The method of any one of embodiments 160-162, wherein the report contains a recommendation for surgical intervention. 164. The method of any one of embodiments 134-163, wherein the method has a false discovery rate of 5% or less. 165. A method of detecting endometriosis in an individual, comprising: a. determining from one of a cervicovaginal fluid sample or a menstrual fluid sample of the individual an expression level of one or more microRNAs (miRs) selected from the group consisting of: miR-1271-5p, miR-4485-3p, miR-125b-2-3p, miR-410-3p, let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-12′7-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p; and b. comparing the expression level to a reference expression level of the one or more miRs; wherein an increased or decreased expression level of the one or more miRs relative to the reference expression level indicates that the subject has endometriosis. 166. The method of embodiment 165, wherein the one or more miRNAs comprise miR-23b-3p, miR-30a-3p/5p, and miR-34a-5p. 167. The method of embodiment 165 or embodiment 166, wherein the reference expression level is obtained from a subject not suffering from a reproductive disorder or not suspected of having the reproductive disorder. 168. The method of embodiment 166, wherein the reproductive disorder is endometriosis. 169. The method of any one of embodiments 165-168, wherein the individual suffers from chronic pelvic pain, infertility, heavy menstrual bleeding, or a combination thereof. 170. The method of any one of embodiments 165-169, wherein the endometriosis is deep infiltrating endometriosis (DIE), superficial peritoneal endometriosis (SPE), or ovarian endometriomas (OE). 171. The method of any one of embodiments 165-170, further comprising determining a methylation profile of one or more CpG sites selected from the CpG sites in Table 4. 172. The method of any one of embodiments 165-171, further comprising determining a measure of bacterial diversity in the biological sample. 173. The method of embodiment 172, wherein the measure of bacterial diversity is an amount of at least one bacterium. 174. The method of embodiment 173, wherein the at least one bacterium is a bacterium in a genus selected from the group consisting of: Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella. 175. The method of any one of embodiments 172-174, further comprising comparing the measure of bacterial diversity to a reference measure of bacterial diversity. 176. The method of any one of embodiments 172-175, wherein the measure of bacterial diversity is a ratio of at least one first bacterium to at least one second bacterium. 177. The method of any one of embodiments 165-176, further comprising determining an amount of Propionibacterium acnes. 178. The method of embodiment 177, further comprising comparing the amount of Propionibacterium acnes to a reference amount of Propionibacterium acnes. 179. The method of any one of embodiments 165-178, wherein the biological sample is disposed in a sample collector. 180. The method of embodiment 179, wherein the sample collector is a pad, a tampon, a vaginal cup, a cervical cap, a menstrual disk, a cervical disk, a sponge, or an interlabial pad. 181. The method of any one of embodiments 165-180, further comprising administering a treatment to the individual for endometriosis. 182. The method of embodiment 181, wherein the treatment is selected from the group consisting of a surgical intervention, administration of therapeutic agent, and a combination thereof. 183. The method of embodiment 182, wherein the therapeutic agent selected from the group consisting of: a hormone, a hormone agonist, a hormone antagonist, an aromatase inhibitor, an anti-inflammatory therapy, an acetyltransferase, a histone deacetylase inhibitor, a phosphodiesterase inhibitor, and a combination thereof. 184. The method of any one of embodiments 165-183, further comprising generating a report based on the expression level of the one or more miRs relative to the reference expression level. 185. The method of embodiment 184, further comprising transmitting the report to a health practitioner. 186. The method of embodiment 184 or embodiment 185, wherein the report contains a recommendation for administering a therapeutic agent. 187. The method of any one of embodiments 184-186, wherein the report contains a recommendation for surgical intervention. 188. The method of any one of embodiments 165-187, wherein the method has a false discovery rate of 5% or less. 189. A method of detecting endometriosis in an individual, comprising: a. determining from a biological sample of the individual an expression level of one or more microRNA that regulates an expression of at least one gene involved in at least one KEGG pathway selected from the group consisting of: ECM-receptor, Adherens junction, Proteoglycans in cancer, TGF-beta signaling, Hippo signaling, MicroRNAs in cancer, Pathways in cancer, Hepatitis B, Glioma, Chronic myeloid leukemia, Bladder cancer, and a combination thereof; and b. comparing the expression level to a reference expression level, wherein an increased or decreased expression level of the one or more microRNA or one or more gene relative to the reference expression level indicates that the subject has endometriosis. 190. The method of embodiment 189, wherein the one or more microRNA is selected from the group consisting of miR-23b-3p, miR-30a-3p/5p, miR-34a-5p, and a combination thereof. 191. The method of embodiment 189, wherein the one or more microRNA is selected from the group consisting of let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p. 192. The method of any one of embodiments 189-191, wherein the one or more gene is selected from the group consisting of TGF-α, TGF-β, progesterone receptor A, progesterone receptor B, estrogen receptor A, E-cadherin, N-cadherin, and a combination thereof. 193. The method of any one of embodiments 189-192, wherein the at least one KEGG pathway is involved with Wnt/JNK/VEGF signaling. 194. The method of any one of embodiments 189-193, wherein the reference expression level is obtained from a subject not suffering from a reproductive disorder or not suspected of having the reproductive disorder. 195. The method of embodiment 194, wherein the reproductive disorder is endometriosis. 196. The method of any one of embodiments 189-195, wherein the individual suffers from chronic pelvic pain, infertility, heavy menstrual bleeding, or a combination thereof. 197. The method of any one of embodiments 189-196, wherein the endometriosis is deep infiltrating endometriosis (DIE), superficial peritoneal endometriosis (SPE), or ovarian endometriomas (OE). 198. The method of any one of embodiments 189-197, further comprising determining a methylation profile of one or more CpG sites selected from the CpG sites in Table 4. 199. The method of any one of embodiments 189-198, further comprising determining a measure of bacterial diversity in the biological sample. 200. The method of embodiment 199, wherein the measure of bacterial diversity is an amount of at least one bacterium. 201. The method of embodiment 200, wherein the at least one bacterium is a bacterium in a genus selected from the group consisting of: Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella. 202. The method of any one of embodiments 199-201, further comprising comparing the measure of bacterial diversity to a reference measure of bacterial diversity. 203. The method of any one of embodiments 199-202, wherein the measure of bacterial diversity is a ratio of at least one first bacterium to at least one second bacterium. 204. The method of any one of embodiments 189-203, further comprising determining an amount of Propionibacterium acnes. 205. The method of embodiment 204, further comprising comparing the amount of Propionibacterium acnes to a reference amount of Propionibacterium acnes. 206. The method of any one of embodiments 189-205, wherein the biological sample is a menstrual fluid. 207. The method of embodiment 206, wherein the menstrual fluid further comprises cervicovaginal fluid. 208. The method of 206 or embodiment 207, wherein the biological sample is collected on a second day of the individual's menstrual cycle. 209. The method of any one of embodiments 206-208, wherein the biological sample is collected on a day of the individual's menstrual cycle where the individual experiences a heavy flow of menstrual fluid. 210. The method of any one of embodiments 189-209, wherein the biological sample is collected prior to administering a treatment to the individual. 211. The method of any one of embodiments 189-209, wherein the biological sample is collected after administering a treatment to the individual. 212. The method of any one of embodiments 189-211, wherein the biological sample is disposed in a sample collector. 213. The method of embodiment 212, wherein the sample collector is a pad, a tampon, a vaginal cup, a cervical cap, a menstrual disk, a cervical disk, a sponge, or an interlabial pad. 214. The method of any one of embodiments 189-213, further comprising administering a treatment to the individual for endometriosis. 215. The method of embodiment 214, wherein the treatment is selected from the group consisting of a surgical intervention, administration of therapeutic agent, and a combination thereof. 216. The method of embodiment 215, wherein the therapeutic agent selected from the group consisting of: a hormone, a hormone agonist, a hormone antagonist, an aromatase inhibitor, an anti-inflammatory therapy, an acetyltransferase, a histone deacetylase inhibitor, a phosphodiesterase inhibitor, and a combination thereof. 217. The method of any one of embodiments 189-216, further comprising generating a report based on the expression level of the one or more miRs relative to the reference expression level. 218. The method of embodiment 217, further comprising transmitting the report to a health practitioner. 219. The method of embodiment 217 or embodiment 218, wherein the report contains a recommendation for administering a therapeutic agent. 220. The method of any one of embodiments 217-219, wherein the report contains a recommendation for surgical intervention. 221. The method of any one of embodiments 217-220, wherein the method has a false discovery rate of 5% or less. 222. A method of treating a subject suspected of having endometriosis comprising: obtaining or having obtained a biological sample from the subject; and performing or having performed an assay on the biological sample to determine if the subject has a microRNA expression signature indicative of endometriosis; and if the subject has the microRNA expression signature indicative of endometriosis, then administering to the subject an intervention, and if the subject does not have the methylation signature indicative of endometriosis, no intervention is administered. 223. The method of embodiment 222, wherein the microRNA expression signature indicative of endometriosis comprises a significantly different expression of one or more microRNAs (miRs) selected from the group consisting of: miR-1271-5p, miR-4485-3p, miR-125b-2-3p, miR-410-3p, let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p, and a combination thereof, relative to expression of the one or more microRNAs in an individual not having endometriosis. 224. The method of embodiment 222, wherein the microRNA expression signature indicative of endometriosis comprises a significantly different expression of one or more microRNAs (miRs) selected from the group consisting of: miR-1271-5p, miR-4485-3p, miR-125b-2-3p, miR-410-3p, and a combination thereof, relative to expression of the one or more microRNAs in an individual not having endometriosis. 225. The method of any one of embodiments 222-224, wherein the performing or having performed the assay on the biological sample to determine if the subject has an microRNA expression signature indicative of endometriosis comprises: extracting or having extracted nucleic acid from the biological sample, and sequencing or having sequenced one or more microRNAs from the extracted nucleic acid. 226. The method of embodiment 225, wherein the nucleic acid is RNA. 227. The method of any one of embodiments 222-226, wherein the intervention is selected from the group consisting of a surgical intervention, a therapeutic intervention, and a combination thereof. 228. The method of embodiment 227, wherein the surgical intervention is selected from the group consisting of: surgical removal of at least a part of an endometriosis lesion, hysterectomy, salpingo-oophorectomy, presacral neurectomy, and laparoscopic uterine nerve ablation. 229. The method of embodiment 227, wherein the therapeutic intervention comprises administration of a therapeutic agent selected from the group consisting of: a hormone, a hormone agonist, a hormone antagonist, an aromatase inhibitor, an anti-inflammatory therapy, an acetyltransferase, a histone deacetylase inhibitor, a phosphodiesterase inhibitor, and a combination thereof. 230. The method of embodiment 229, wherein the hormone is selected from the group consisting of estrogen, progestin, androgen, and gonadotropin-releasing hormone (Gn-RH). 231. The method of embodiment 229 or embodiment 230, wherein the hormone is a synthetic hormone. 232. The method of embodiment 229, wherein the hormone agonist or antagonist is a gonadotropin-releasing hormone (Gn-RH) agonist or Gn-RH antagonist. 233. A method of preserving cells from a menstrual fluid sample, the method comprising disposing the menstrual fluid sample comprising the cells in a preservation solution to form a mixture of the menstrual fluid sample comprising the cells and the preservation solution. 234. The method of embodiment 233, further comprising contacting the cells in the menstrual fluid sample with an antibody that binds to a cell surface antigen of a target cell in the cells in the menstrual fluid sample. 235. The method of embodiment 234, wherein the antibody is attached to a solid support. 236. The method of embodiment 235, wherein the solid support is a bead. 237. The method of embodiment 236, wherein the bead is a magnetic bead. 238. The method of any one of embodiments 234-237, wherein the antibody is conjugated to a detectable marker. 239. The method of embodiment 238, wherein the detectable marker is a fluorophore. 240. The method of any one of embodiments 234-239, wherein the target cell is selected from the group consisting of: an endothelial cell, an epithelial cell, a leukocyte, a mesenchymal cell, and a combination thereof. 241. The method of any one of embodiments 234-240, wherein the target cell is an endothelial cell. 242. The method of embodiment 241, wherein the cell surface antigen is selected from the group consisting of: CD31/PECAM-1, CD34, CD36/SR-B3, CD39, CD44, CD47, CD54/ICAM-1, CD61, CD62E, CD62P, CD80, CD86, CD93, CD102, CD105, CD106, CD112, CD117, ESAM, Endomucin, CXCL16, CD121a, CD141, CD142, CD143, CD144, CD146, CD147, CD151, CD160, CD201, CD213a, CD248, CD309, ADAMs 8, ADAMs 9, ADAMs 10, ADAMs 11, ADAMs 12, ADAMs 13, ADAMs 14, ADAMs 15, ADAMs 16, ADAMs 17, ADAMs 33, ADAMTS-13, ADAMTS-18, VWF, TEM8, NOTCH, and KLF4. 243. The method of any one of embodiments 234-240, wherein the target cell is an epithelial cell. 244. The method of embodiment 243, wherein the cell surface antigen is select from the group consisting of: Epithelial cell adhesion molecule (EpCAM), E-cadherin, and CD326. 245. The method of any one of embodiments 234-240, wherein the target cell is a leukocyte. 246. The method of embodiment 245, wherein the cell surface antigen is CD45. 247. The method of any one of embodiments 234-240, wherein the target cell is a mesenchymal cell. 248. The method of embodiment 247, wherein the cell surface antigen is selected from the group consisting of N-cadherin, OB-cadherin, alpha-5 beta-1 integrin, alpha-V beta-6 integrin, and syndecan-1. 249. The method of any one of embodiments 234-248, further comprising isolating the target cell from the menstrual fluid sample. 250. The method of embodiment 249, wherein the isolating comprises fluorescent activated cell sorting (FACS), magnetic activated cell sorting, or a combination thereof. 251. The method of any one of embodiments 234-248, further comprising removing the target cell from the menstrual fluid sample. 252. The method of any one of embodiments 233-251, wherein at least 20%, at least 30%, at least 40%, at least 50%, at least 60%, at least 70%, at least 80%, or at least 90% of the cells in the menstrual fluid sample are intact. 253. The method of any one of embodiments 233-252, wherein at least 20%, at least 30%, at least 40%, at least 50%, at least 60%, at least 70%, at least 80%, or at least 90% of the cells in the menstrual fluid sample are viable. 254. The method of any one of embodiments 233-253, wherein the osmolality of the preservation solution is from about 310 to about 410 mOsm kg-1. 255. The method of any one of embodiments 233-252, wherein the osmolality of the preservation solution is from about 95 to about 210 mOsm kg-1. 256. The method of any one of embodiments 233-255, wherein the volume of preservation solution is from about 5 ml to about 10 ml. 257. The method of any one of embodiments 233-255, wherein the volume of the preservation solution is about 7.5 ml. 258. A menstrual fluid cell sample comprising: one or more cells from a menstrual fluid sample; and a preservation solution. 259. The sample of embodiment 258, wherein the one or more cells are selected from the group consisting of an endothelial cell, an epithelial cell, a leukocyte, a mesenchymal cell, and a combination thereof. 260. The sample of embodiment 259, wherein the epithelial cell is an endometrial epithelial cell. 261. The sample of any one of embodiments 258-260, wherein the preservation solution comprises a precipitating agent. 262. The sample of embodiment 261, wherein the precipitating agent is selected from the group consisting of: 5-(4-dimethyl)amino benzylidene rhodanine, sulfosalicyclic acid, lithium chloride, and lithium hydroxide. 263. The sample of any one of embodiments 258-262, wherein the preservation solution comprises a lower alcohol. 264. The sample of embodiment 263, wherein the lower alcohol is selected from the group consisting of: methanol, ethanol, n-propanol, isopropanol, n-butanol, and isobutanol (2-methylpropan-1-ol). 265. The sample of any one of embodiments 258-264, wherein the preservation solution comprises a chaotrope. 266. The sample of embodiment 265, wherein the chaotrope is selected from the group consisting of: guanidine hydrochloride, guanidine thiocyanate, potassium thiocynanate, sodium thiocyanate, and urea. 267. The sample of any one of embodiments 258-266, wherein the preservation solution comprises a chelating agent. 268. The sample of embodiment 267, wherein the chelating agent is selected from the group consisting of: diethylenetriaminepentaacetic acid (DTPA), ethylenediaminetetraacetic acid (EDTA), ethylene glycol tetraacetic acid (EGTA), trans-1,2-diaminocyclohexane-N,N,N′,N′-tetraacetic acid (CDTA), 1,2-bis(2-aminophenoxy)ethane-N,N,N′,N′-tetraacetic acid (BAPTA), 1,4,7,10-tetraazacyclododecane-1,4,7,10-tetraacetic acid (DOTA), N-(2-hydroxyethyl)ethylenediamine-N,N′,N′-triacetic acid, and nitrilotriacetic acid (NTA). 269. The sample of any one of embodiments 258-268, wherein the preservation solution comprises a reducing agent. 270. The sample of embodiment 269, wherein the reducing agent is selected from the group consisting of: 2-mercaptoethanol, thiosulfate, TCEP (tris-(2-carboxyethyl) phosphine), dithiothreitol, and dithioerythritol. 271. The sample of any one of embodiments 258-270, wherein the preservation solution comprises a pH buffer. 272. The sample of embodiment 271, wherein the pH buffer is selected from the group consisting of: citric acid, tartaric acid, malic acid, sulfosalicylic acid, sulfoisophtalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), IVIES (2-(N-morpholino)ethanesulfonic acid), MOPS (3-(N-morpholine)propanesulfonic acid), MOPSO (3-morpholine-2-hydroxypropanesulfonic acid), PIPES (1-4-piperazinediethanesulfonic acid), TAPS (N-[tris(hydroxymethyl)methyl]-3-aminopropanesulfonic acid), TAPSO (2-hdyroxy-3-[tris(hdyroxymethyl)methylamino]-1-propanesulfonic acid), TES (N-[tris(hydroxymethyl)methyl]-2-aminoethanesulfonic acid), bicine (N,N-Bis(2-hdyroxyethyl)glycine), tricine (N-[Tris(hydroxymethyl)methyl]glycine), tris (tris(hydroxymethyl)aminomethane), and bis-tris (2-[Bis(2-hdyroxyethyl)amino]-2-(hdyroxymethyl)-1,3-propanediol). 273. The sample of any one of embodiments 258-272, wherein the preservation solution comprises a surfactant. 274. The sample of any one of embodiments 258-273, wherein the osmolality of the preservation solution is from about 310 to about 410 mOsm kg-1. 275. The sample of any one of embodiments 258-273, wherein the osmolality of the preservation solution is from about 95 to about 210 mOsm kg-1. 276. The sample of any one of embodiments 258-275, wherein the preservation solution does not comprise a fixative. 277. The sample of any one of embodiments 258-276, further comprising a menstrual fluid. 278. The sample of any one of embodiments 258-277, further comprising a cervicovaginal fluid. 279. The sample of any one of embodiments 258-278, wherein the volume of the menstrual fluid sample is from about 100 μl to about 1 ml. 280. The sample of any one of embodiments 258-279, wherein at least 20%, at least 30%, at least 40%, at least 50%, at least 60%, at least 70%, at least 80%, or at least 90% of the cells in the sample are intact. 281. The sample of any one of embodiments 258-280, wherein at least 20%, at least 30%, at least 40%, at least 50%, at least 60%, at least 70%, at least 80%, or at least 90% of the cells in the sample are viable. 282. The sample of any one of embodiments 258-281, further comprising a bacterial cell, a yeast cell, a spermatozoa, or a combination thereof. 283. The sample of any one of embodiments 258-282, further comprising a portion of a sample collector. 284. The sample of embodiment 283, wherein the sample collector is a pad, a tampon, a vaginal cup, a cervical cup, a menstrual disk, a cervical disk, a sponge, or an interlabial pad.

### EXAMPLES

### Example 1

### RNA-Seq Timecourse Data

RNA-seq libraries were assessed for quality metrics by looking at the number of overall reads aligning to the human transcriptome. Serial time course samples (both menstrual (MB) and cervicovaginal samples (CV)) were analyzed from women by collecting a tampon-based specimen on every day of a 28-day cycle, including a peripheral whole blood (WB) draw on heavy flow day. A device as shown in FIG. 5 was used to collect the samples. RNAgard was used as a preservation solution for the sample. RNA was sequenced from 244 samples from 27 participants (171 CV, 46 MB, and 27 WB). Typically, for high quality genomic analysis, at least 70% of sequencing reads should align to the reference transcriptome. This is an industry standard and independent of sequencing platform used. Menstrual and whole blood both show similar robustness in percentage of reads aligning to the transcriptome (mean=93.8%; StDev=4.6% for menstrual fluid and mean=95.5%; StDev=1.1% for whole blood). These percentages are well beyond the 70% threshold for genomic analysis, demonstrating the high-quality capture of nucleic acids from menstrual fluid.

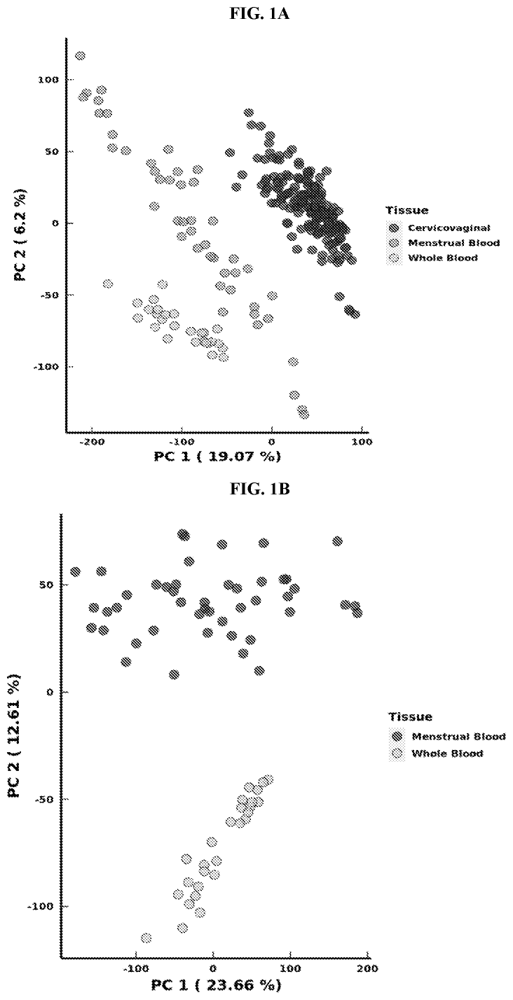

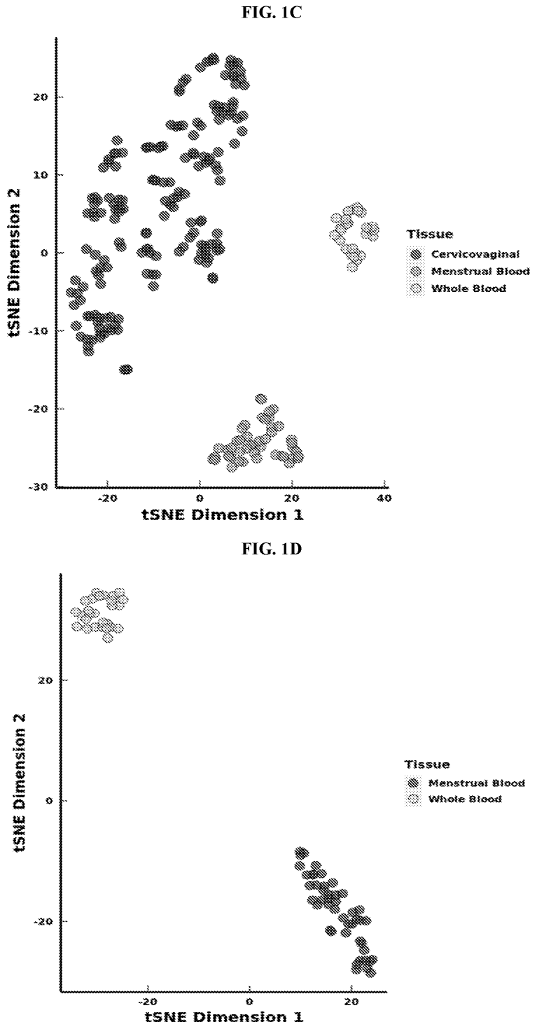

To assess inter- and intra-sample variation from participants, an unsupervised principal component analysis (PCA) was performed and then t-SNE, a non-linear dimensional reduction technique that prefers maintaining local versus global structure, was utilized to visualize all variation within two dimensions (FIG. 1C-1D). There was a tight clustering of menstrual samples, indicating a high level of reproducibility of this sample type, when compared to CV, which demonstrates a high degree of variability over the menstrual cycle, and WB, as well as a distinct clustering of menstrual samples that represent approximately 800 differentially expressed genes (FIG. 1A-1B).

To better understand the value that the menstrualome offers over whole blood specimens, total RNA transcription in whole blood and in menstrual fluid collected from the same patient, at the same time, on the first three days of menstruation were compared. Menstrual fluid, as above, was a tampon-based specimen, wherein a device as shown in FIG. 5 was used to collect the samples. Whole blood specimens were collected using venipuncture. By performing a differential analysis between each matched sample on each day of menstruation, genes were identified that were differentially expressed in menstrual fluid but not in whole blood. Interestingly, on the first day of menstruation, very little difference in the relative abundance of gene transcripts was observed between menstrual fluid and whole blood. However, on the second day of menstruation, which is often referred to as “heavy flow day”—when a woman sheds the majority of her endometrial lining—over 800 differentially expressed genes that represented a unique genomic profile of menstrual fluid were identified (FIG. 1A-1B).

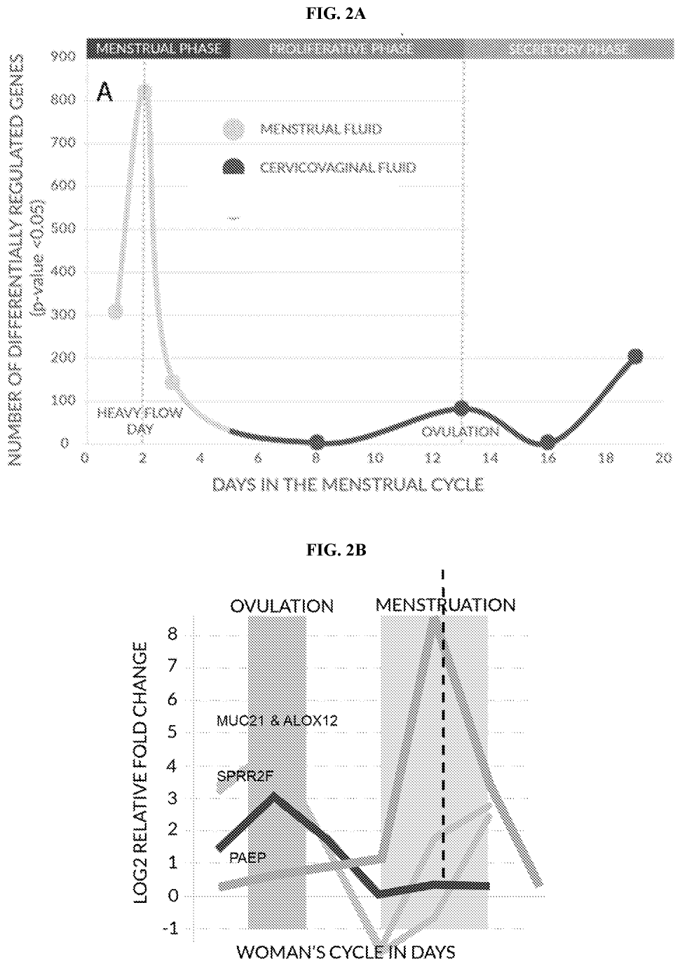

In a time-course analysis, several genes expressed in a variety of reproductive tissues (expression from tissue specific genes of cervical/vaginal cells, ovarian/fallopian tube cells, and endometrial cells) were identified. The relative abundance of each of these markers was compared over the course of a woman's menstrual cycle. On the second day of menstruation, the gene signals for endometrial tissue were found to be greatly overrepresented in menstrual samples (FIG. 2B). On non-menstruation days, samples collected during ovulation gave the highest yield of RNA and DNA for cervicovaginal samples, and an enrichment of ovarian and fallopian tube specific genes during ovulation was found.

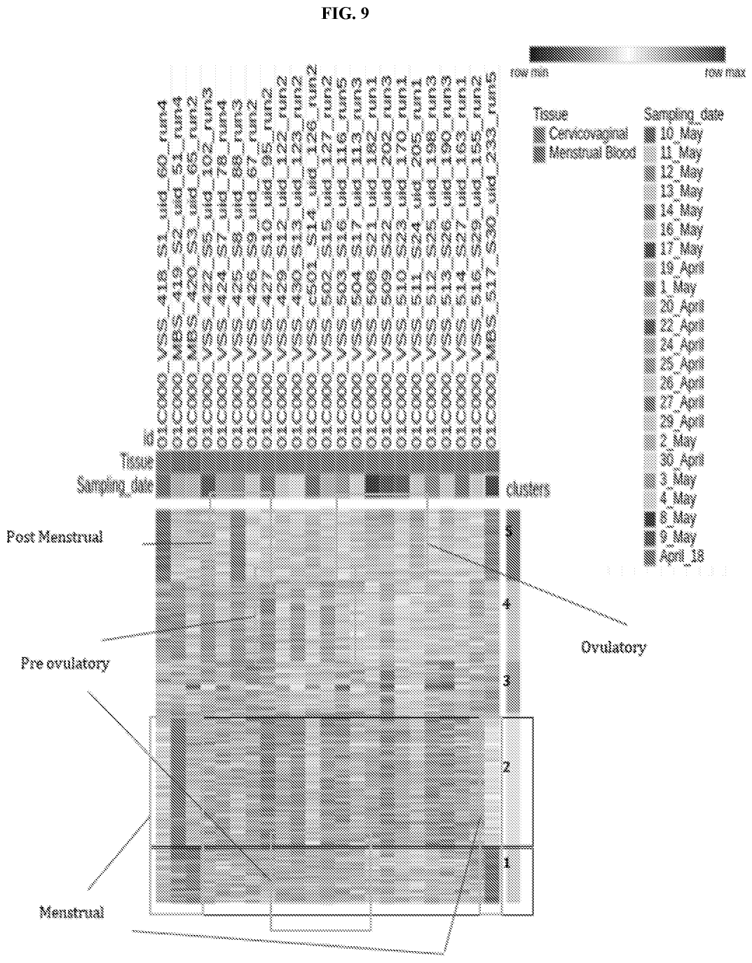

Normalized gene expression values for patient C000 for a time series of samples spanning April 18 to May 17 were hierarchical clustered using a K-means clustering algorithm on Morpheus, an open-source software program developed by the Broad Institute. As depicted in FIG. 9, there are 5 main clusters from this time series data where overexpression and underexpression is seen as samples move chronologically through the time series. In cluster 2, overexpression of genes was detected in the menstrual samples, while in cluster 2, an underexpression of certain genes was detected in the menstrual samples. Post menstrual phase an overexpression of genes in cluster 5, Pre-ovulatory an overexpression in cluster 1 and an underexpression in cluster 4 was detected. And finally in the Ovulatory phase an overexpression of genes in cluster 5 was detected.

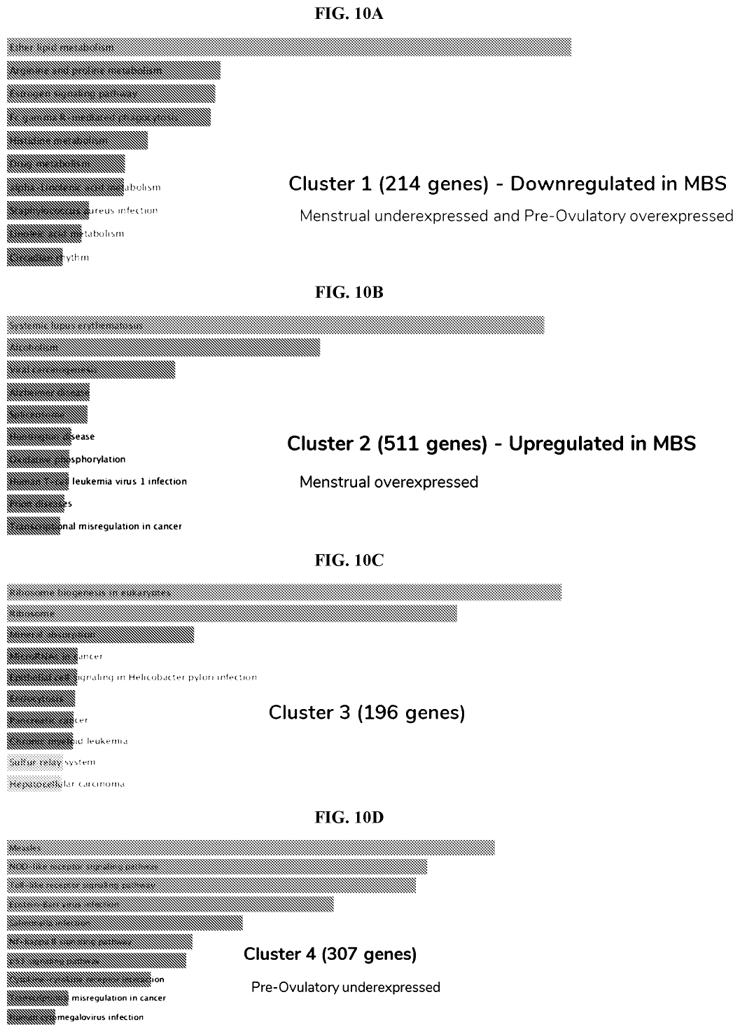

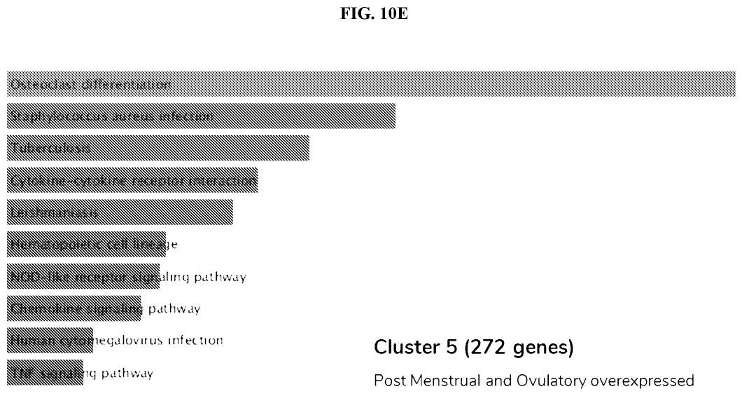

A Kegg pathway analysis was performed on the list of genes from each cluster, as depicted in FIG. 10A-10E. Cluster 1 contained genes downregulated in menstrual bleeding and showed KEGG pathways regulated included ether lipid metabolism, arginine and proline metabolism, estrogen signaling pathway, Fc gamma R-mediated phagocytosis, histidine metabolism, drug metabolism, alpha-Lineolenic acid metabolism, Staphylococcus aureus infection, linoleic acid metabolism, and circadian rhythm. The KEGG pathways regulated in cluster 2 were associated with system lupus erythematosus, alcoholism, viral carcinogenesis, Alzheimer disease, spliceosome, Huntington disease, oxidative phosphorylation, human T-cell leukemia virus 1 infection, prion diseases and transcriptional misregulation in cancer. The KEGG pathways regulated in cluster 3 were associated with ribosome biogenesis in eukaryotes, ribosome, mineral absorption, microRNAs in cancer, epithelial cell signaling in Helicobacter pylori infection, endocytosis, pancreatic cancer, chronic myeloid leukemia, sulfur relay system and hepatocellular carcinoma. The KEGG pathways regulated in cluster 4 were associated with measles, NOD-like receptor signaling pathway, Toll-like receptor signaling pathway, Epstein-Barr virus infection, Salmonella infection, NF-kappa B signaling pathway, p53 signaling pathway, cytokine-cytokine receptor interaction, transcriptional misregulation in cancer, and human cytomegalovirus infection. The KEGG pathways regulated in cluster 5 were osteoclast differentiation, staphylococcus aureus infection, tuberculosis, cytokine-cytokine receptor interaction, leishmaniasis, hematopoietic cell lineage, NOD-like receptor signaling pathway, chemokine signaling pathway, human cytomegalovirus infection, and TNF signaling pathway.

### Example 2

### Collection of Whole Blood, Cervicovaginal, and Menstrual Samples

Whole blood, cervicovaginal and menstrual samples were collected from women with suspected endometriosis (n=19), healthy women (n=55), and women with polycystic ovarian syndrome (PCOS) (n=5) and both epigenetic regulation (small RNAs and DNA methylation), as well as RNA transcriptional sequencing and bacterial 16s sequencing were analyzed. Menstrual and cervicovaginal samples were collected using a sample collection system as illustrated in FIG. 5, FIG. 6A-6D, 7A-7C and 8A-8C, and whole blood samples were collected through routine venipuncture. Nucleic acids were extracted, and sequencing libraries prepped using Illumina reagents and sequenced on Illumina's MiSeq, NextSeq550, and HiSeq4000 sequencing machines to compare the performance of each sample type for the detection of endometriosis. Patients were classified as described in Table 1A. Of the 19 patients with a confirmed diagnosis, tampons were collected from five prior to surgery and tampons from 14 post-surgery (these were not paired samples and pre- and post-surgery tampon collections were from different participants). The menstrual cycle samples were collected at day 2 of the menstrual cycle. This allowed assessment of the data from the lens of pre-surgery genomic signals vs post-surgery genomic signals in the populations of interest. Staging, classification and anatomical location of disease for patients was also noted. Three of the post-surgery samples failed quality control metrics and were not included in the analysis. For the healthy population, women were recruited from the community who had never been given a diagnosis of a reproductive disorder. Measurements of reproductive hormones (anti-Mullerian hormone (AMH), estradiol, follicular stimulating hormone (FSH), luteinizing hormone (LH), and thyroid stimulating hormone (TSH)), documented symptoms of classical endometriosis presentation, and symptoms of vaginal infections were used to categorize these women into further sub-classifications of “truly healthy” and “suspected unhealthy” (Table 1A). In summary, tampons collected from five “truly healthy” women to five pre-surgery and 11 post-surgery endometriosis patients were compared.

TABLE 1A
Criteria used for categorization of “truly healthy” or “suspected unhealthy”
Patients who present at Nezhat Clinic

| Suspected Endometriosis | | Suspected PCOS |
|---|---|---|
| Laparoscopic Surgery | | Ultrasound + |
| | | AMH + Self- |
| | | reported |
| Endometriosis patients, | No Endometriosis | PCOS |
| confirmed via surgery | Found | Patients |

TABLE 1B
How a healthy patient is recruited
‘Healthy’ Patients Recruited Through Community Gatherings

| | | |
|---|---|---|
| Labcorp readings for AMH, Estradiol, FSH, LH, | Self Reported answers in NGJ | 16 s bacterial sequencing |
| Progesterone, TSH, T4 | questionnaire | performed at NGJ |

| | | | |
|---|---|---|---|
| 1 | 2 | 3 | |
| 1) Labcorp values within normal | 1) 1 or 2 non-critical Labcorp | 1) 1 or 2 Critical Labcorp values | 1) self reported yeast or bacterial |
| reference range | values which are borderline or out | which are out of range | infection within the last 3 months |
| 2) no self reported red flags | of range | 2) 3 or more self reported | 2) self reported antibiotic use within |
| 3) no family history of endo | 2) 1 or 2 self reported symptoms | symptoms of reproductive disorder | the last 3 months |
| | of reproductive disorder | | 3) abnormal vaginal flora detected, |
| | 3) family history of endo | | indicative of BV |

Detection of miRNA Markers

To explore local miRNA signaling and intracellular miRNA signaling, miRNAs in menstrual fluid were isolated and sequenced. A small sub-cohort of patients (five with endometriosis and five truly healthy women) was analyzed to examine differential miRNA expression. The samples for women with endometriosis were collected prior to surgery and surgical confirmation was given for all endometriosis suspected patients. A differential expression analysis was performed on normalized miRNA sequencing from menstrual fluid. 49 significantly (p-value<0.05 at a FDR<0.05) dysregulated miRNAs were detected in endometriosis pre-surgery patients compared to truly healthy women's menstrual fluid (Table 2). Ten of these 49 markers individually had an area under the curve (AUC) of 0.95 or greater, at a false discovery rate of 5%. When the miRNA profiles of pre-surgery patients were compared to post-surgery collected tampons, it was found that miRNA expression in post-surgery endometriosis subjects did not differ from miRNA expression in healthy individuals. This is not to suggest that these patients were cured, as there is currently no cure for endometriosis, but data suggest the ability to detect disease activation. Post-surgery tampon samples were collected 3 months to 2 years post-surgery and no recurrence of disease was present within these patients as far as two years post-surgery. The findings illustrated the utility of miRNA for monitoring the efficacy of surgical or other interventions, including the efficacy of therapeutics to reduce disease activity.

TABLE 2
Significantly dysregulated miRNAs in endometriosis pre-surgery
patients compared to truly healthy women's menstrual fluid

| | | Area Under | |
|---|---|---|---|
| miRNA | Disease Implication | the Curve | Sample type |
| miR-1271-5p | Endometrial Cancer | AUC > 0.95 | Eutopic Endometrium |
| miR-4485-3p | Mitochondrial function & | | Other |
| | Breast Cancer | | |
| let-7c-5p | Endometriosis | | Stromal Cells |
| miR-100-5p | | | Peritoneal Fluid; Eutopic |
| miR-149-5p | | | Peritoneal Fluid |
| miR-193b-3p | | | Eutopic |
| miR-221-5p | | | Eutopic and Ectopic |
| miR-363-3p | | | Stromal Cells |
| miR-99a-5p | | | Peritoneal Fluid; Eutopic |
| miR-99b-5p | | | Serum |
| let-7e-5p, miR-10a-5p, miR-10b- | | AUC < 0.9 | Serum; Eutopic; Ectopic, |
| 5p, miR-125b-5p, miR-127-3p, | | | Peritoneal Fluid |
| miR-132-3p, miR-141-3p, miR- | | | |
| 142-5p, miR-143-3p, miR-144- | | | |
| 5p, miR-145-5p, miR-152-3p, | | | |
| miR-16-2-3p, miR-17-3p, miR- | | | |
| 195-5p, miR-196b-5p, miR-199a- | | | |
| 3p/199b-3p, miR-200a-3p, miR- | | | |
| 200c-3p, miR-203a-3p, miR-205- | | | |
| 5p, miR-21-3p, miR-21-5p, miR- | | | |
| 22-3p, miR-222-3p, miR-224-5p, | | | |
| miR-23b-3p, miR-27b-3p, miR- | | | |
| 28-3p, miR-30a-3p, miR-30a-5p, | | | |
| miR-34a-5p, miR-34c-5p, miR- | | | |
| 365a-3p/365b-3p, miR-375, miR- | | | |
| 409-3p, miR-98-5p | | | |
| miR-125b-2-3p | Endometrial & Ovarian | | Serum and Eutopic |
| | Cancer, Miscarriage | | |
| miR-410-3p | Endometrial Cancer | | Eutopic |

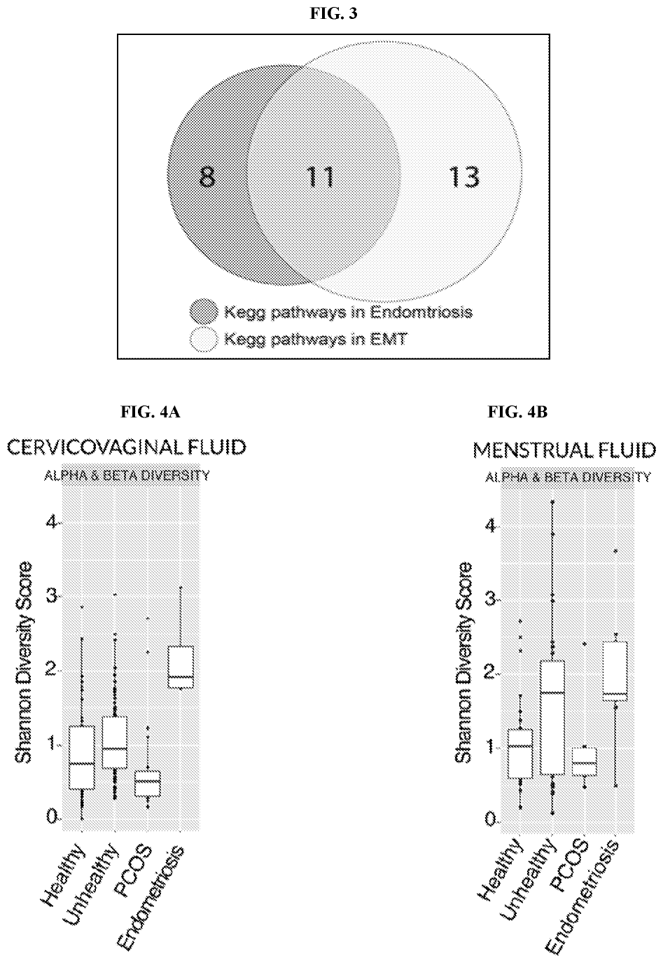

The development of methods that can stratify patients according to DIE, SPE, OE, aromatase expression, retinoic acid imbalance, and skewed estrogen and progesterone receptors may predict a patient's response to an intervention. In order to understand relevant pathways involved in endometriosis within the data provided herein, pathway analysis was performed on the 49 miRNAs using mirPath v.3, and results were compared to 377 genes involved in epithelial mesenchymal trans-differentiation (EMT) and their respective Kegg pathways known as the EMT database (dbEMT). A total of 11 pathways were shared between these candidate markers for endometriosis and EMT (FIG. 3). These include the Hippo signaling pathway, TGF-beta, as well as pathways involved with Wnt/JNK/VGEF signaling, key biological pathways involved in the pathology of endometriosis.

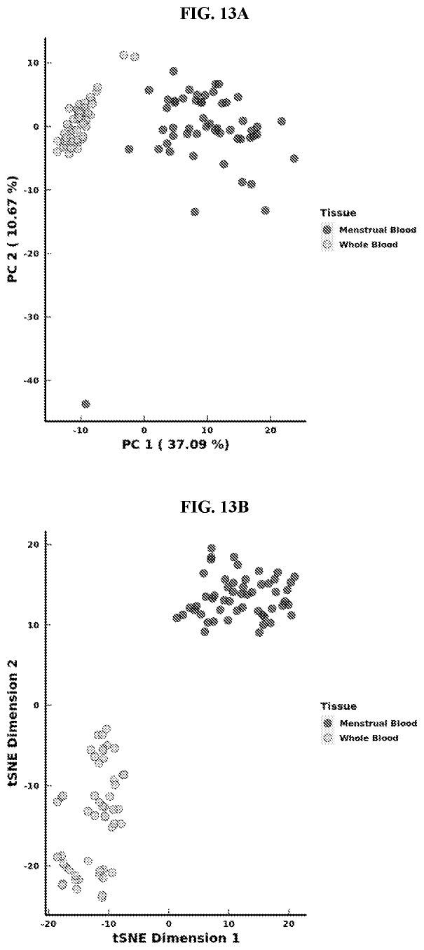

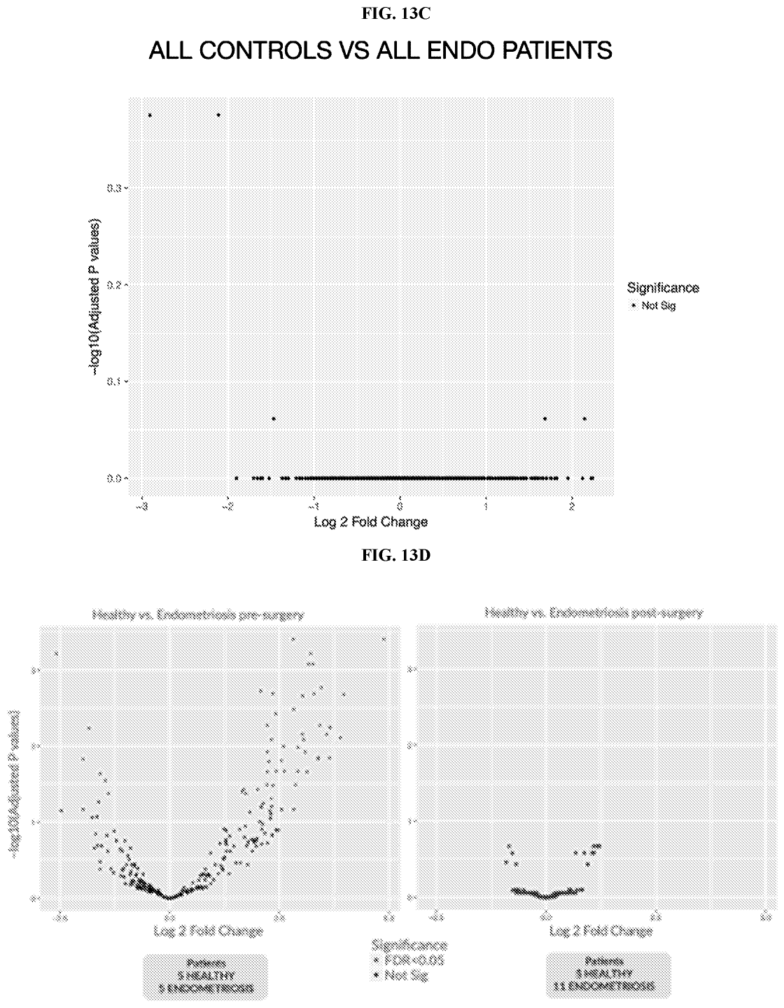

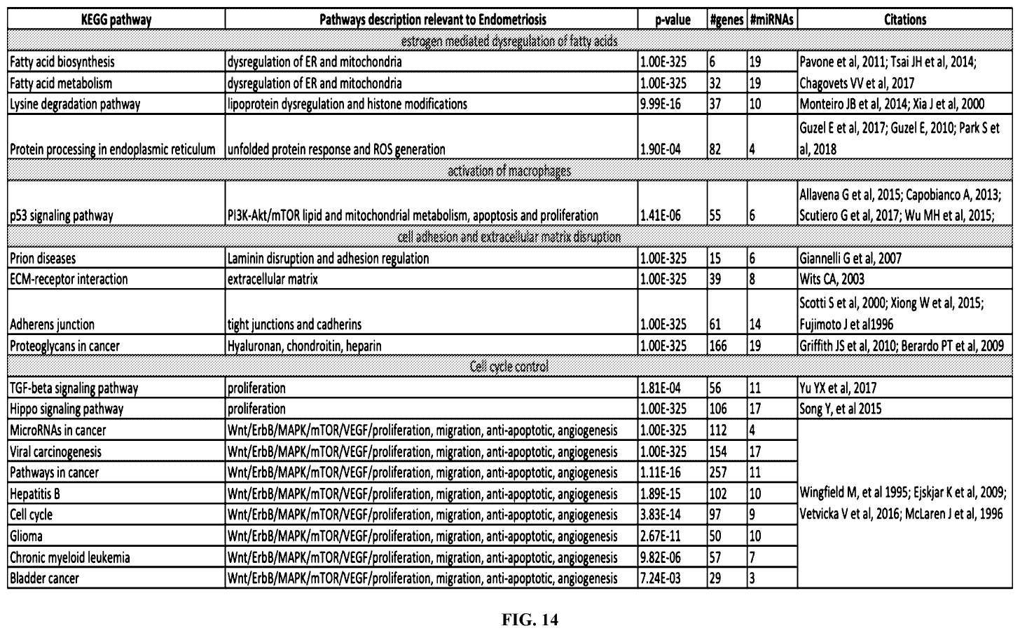

104 samples were collected of patients with healthy or endometriosis conditions, including 53 samples of menstrual blood and 51 samples of whole blood. Samples were isolated and sequenced as described about. When analyzing the dimensionality by PCA plots or tSNE plots, menstrual blood and whole blood were found to cluster together (FIGS. 13A-13B), showing clear differentiation by tissue type.

When the transcription patterns of menstrual blood from all healthy and all endometriosis patients were compared together, there were no significantly differentially regulated miRNAs, as depicted in FIG. 13C. However, when menstrual blood samples were segregated into pre-surgery and post-surgery, there were 49 genes significantly differentially expressed between patients.

These candidate markers were also assessed for their potential to molecularly/genomically classify disease by looking at specific miRNA targets that coordinate epigenetic signaling to specific genes associated with key pathways involved in endometriosis. Using TarBase v.867, the 49 candidate biomarkers were queried for experimentally supported gene interactions and were compared to known cellular markers of endometriosis. Three miRNAs (miR-23b-3p, miR-30a-3p/5p, and miR-34a-5p) were identified that had a high degree of experimental support for many genes involved in endometriosis, including INK1-3 and LATS1 (Hippo signaling). The miRNAs showed evidence of interaction with TGF-α, TGF-β, progesterone receptors A and B, estrogen receptors A and B, as well as both E-cadherin and N-cadherin (Table 3). FIG. 3 shows that 11 Kegg pathways are shared between endometriosis and EMT. FIG. 14 details these 11 pathways, indicates their relevance to endometriosis, the significance (p-value) of these pathways in the 49 miRNAs provided herein, as well as the number of miRNAs from the data provided here that support each pathway. These candidate markers may help to stratify patients by molecular/genomic classification, identify aromatase activity, ratio of progesterone receptors-A vs receptor-B, and ratio of N-cadherin vs E-cadherin expression and estrogen receptor A vs estrogen receptor B.

Further, the miRNA levels differ from those described in previous studies and tissue types, as depicted in Table 3. This data demonstrates the differential expression in menstrual fluid between endometriosis patients and healthy patients, in contrast to the values found in previous comparisons of ectopic endometrial tissue from endometriosis patients versus eutopic endometrial tissue from healthy individuals, ectopic endometrial tissue from endometriosis patients vs eutopic endometrial tissue from endometriosis patients, as well as eutopic endometrial tissue from endometriosis patients vs eutopic endometrial tissue from healthy individuals. Differential expression is shown as log10 foldchange. This demonstrates that menstrual blood displays differential expression than other tissues.

TABLE 3
miRNA levels in menstrual blood versus previous studies in other tissue

| | | | Plasma | | Ectopic | | Ectopic | | Eutopic | |
|---|---|---|---|---|---|---|---|---|---|---|
| | | Menstrual | Endo vs | | Endo vs | | Endo vs | | Endo vs | |
| | | Expn: | Plasma | | Eutopic | | Eutopic | | Eutopic | |
| | | Endo vs | Healthy | | Healthy | | Endo | | Healthy | |
| | Published disease | Healthy | Log10 | | Log10 | | Log10 | | Log10 | |
| miRNA | association | log10 FC | FC | Ref | FC | Ref | FC | Ref | FC | Ref |
| hsa-let-7c-5p | Endometriosis | 0.849 | 0.1 | 6 | −0.01 | 4 | | | | |
| hsa-let-7e-5p | Endometriosis | 0.767 | −0.0 | 6 | −0.13 | 4 | | | | |
| hsa-miR-100-5p | Endometriosis pain and | 0.670 | −.14 | 5 | 0.4 | 2 | 0.39 | 3 | | |
| | inflammation: mTOR, | | | | | | | | | |
| | IG1R | | | | | | | | | |
| hsa-miR-10a-5p | Endometriosis | 0.669 | | | 0.269 | 2 | | | | |
| hsa-miR-10b-5p | Endometriosis | 0.523 | | | 0.37 | 2 | | | | |
| hsa-miR-125b-2-3p | Endometriosis | 0.851 | ? | 8 | | | | | | |
| hsa-miR-125b-5p | Endometriosis | 0.668 | 2 | 7 | | | 0.59 | 9 | | |
| hsa-miR-127-3p | Endometriosis | 0.929 | | | | | 0.91 | 11 | | |
| hsa-miR-1271-5p* | Endometrial cancer; | 0.781 | | | | | | | | |
| | Cardiovascular disease; | | | | | | | | | |
| | anxiety | | | | | | | | | |
| hsa-miR-132-3p | Endometriosis | 0.680 | | | | | −0.32 | 3 | | |
| hsa-miR-141-3p | Endometriosis | 0.911 | | | −0.13 | 2 | −0.7 | 9 | | |
| hsa-miR-142-5p | Endometriosis | −0.553 | | | | | −0.68 | 9 | | |
| hsa-miR-143-3p | Endometriosis | 0.507 | | | | | 0.42 | 3 | | |
| hsa-miR-144-5p | Endometriosis; infertility | −0.418 | | | | | | | −0.33 | 1 |
| hsa-miR-145-5p | Endometriosis | 1.017 | −.1, 1.6 | 5, 6 | 0.22 | 4 | 0.399 | 3 | −0.23 | 1 |
| hsa-miR-149-5p | Endometriosis | 1.041 | | | | | | | | |
| hsa-miR-152-3p | Endometriosis | 0.880 | | | 0.29 | 2 | | | | |
| hsa-miR-16-2-3p* | Endometriosis | −0.777 | | | | | | | | |
| hsa-miR-17-3p | Endometrial disorders | −0.593 | −.32 | 6 | 0.34 | 2 | | | | |
| hsa-miR-193b-3p* | Polycystic Ovarian | 0.984 | | | | | | | | |
| | Syndrome | | | | | | | | | |
| hsa-miR-195-5p | Endometriosis | 1.021 | 18.67 | 12 | | | | | | |
| hsa-miR-196b-5p | Endometriosis | 0.787 | | | | | −1.4 | 3 | | |
| hsa-miR-199a- | Endometriosis | 0.709 | | | 0.16 | 4 | 0.44 | 3 | | |
| 3p/199b-3p | | | | | | | | | | |
| hsa-miR-200a-3p | Endometriosis | 0.698 | | | | | −1.2 | 3 | | |
| hsa-miR-200c-3p | Endometriosis | 0.911 | | | | | −1.2 | 3 | | |
| hsa-miR-203a-3p | Endometriosis | 1.100 | | | | | −2.57 | 11 | | |
| hsa-miR-205-5p | Endometriosis; infertility | 1.077 | | | −1.5 | 13 | | | | |
| hsa-miR-21-3p | Endometriosis | 0.497 | | | 0.43 | 4 | | | | |
| hsa-miR-21-5p | Endometriosis | 0.670 | .07 | 6 | | | | | | |
| hsa-miR-22-3p | Endometriosis | −0.440 | >0.3 | 10 | | | | | | |
| hsa-miR-221-5p | Endometriosis | 0.919 | | | | | 0.3 | 3 | | |
| hsa-miR-222-3p | Endometriosis | 0.603 | | | | | | | −0.22 | 1 |
| hsa-miR-224-5p | Endometriosis | 1.194 | | | | | −1.15 | 11 | | |
| hsa-miR-23b-3p | Endometriosis | 0.699 | <−0.3 | 10 | | | | | | |
| hsa-miR-27b-3p | Endometriosis | 0.954 | | | | | | | 0.29 | 14 |
| hsa-miR-28-3p | Endometriosis | 0.625 | | | | | 0.49 | 3 | | |
| hsa-miR-30a-3p* | Endometrial cancer | 0.731 | | | | | | | | |
| hsa-miR-30a-5p | Endometriosis | 0.728 | 0.105 | 6 | | | −0.72 | 11 | | |
| hsa-miR-34a-5p | Endometriosis | 1.171 | | | | | 0.4 | 3 | | |
| hsa-miR-34c-5p | Endometriosis | 1.031 | | | | | −0.57 | 9 | | |
| hsa-miR-363-3p | Endometriosis | −0.475 | | | | | −0.88 | 11 | | |
| hsa-miR-365a- | Endometriosis | 1.097 | | | | | 0.496 | 9 | | |
| 3p/365b-3p | | | | | | | | | | |
| hsa-miR-375 | Endometriosis | 0.879 | | | | | −1.9 | 3 | | |
| hsa-miR-409-3p | Endometriosis | 0.862 | <−0.3 | 10 | | | | | | |
| hsa-miR-410-3p* | Endometrial cancer | 0.943 | | | | | | | | |
| hsa-miR-4485-3p | Endometriosis | 1.470 | 0.34 | 6 | | | | | | |
| hsa-miR-98-5p | Endometriosis | 0.709 | <−0.3 | 10 | | | | | | |
| hsa-miR-99a-5p | Endometriosis | 0.991 | | | 0.4 | 2 | 0.43 | 3 | | |
| hsa-miR-99b-5p | Endometriosis | 0.967 | 0.12 | 6 | | | 0.73 | 9 | | |

*not previously implicated in endometriosis

### Detection of Methylation Markers

Using the same patients from the above miRNA analysis, DNA methylation patterns of endometriosis was also examined using Illumina's EPIC 850k methylation array. Methylation signatures were mapped to the genome and compared normalized intensity values between pre-surgery collected menstrual fluid from endometriosis patients and menstrual fluid from truly healthy individuals. In the initial sample set, over one thousand CpG methylation sites were identified that were either hypo- or hyper-methylated compared to menstrual fluid from healthy participants. Table 4 shows 370 of the CpG sites with the most significantly different methylation status between individuals with endometriosis and healthy individuals. Interestingly, in patients with endometriosis, a higher percentage of hypo-methylated sites fell within shores—CpG island flanking regions of the genome that are highly dynamic and implicated in many downstream regulatory functions and diseases.

TABLE 4
CpG sites showing hypo or hyper methylation in individuals
with endometriosis compared to healthy individuals

| EPIC array | | | | |
|---|---|---|---|---|
| CpG ID | chromesome | position | strand | pval |
| cg03654487 | chr9 | 12685429 | − | 1.80E−05 |
| ch.5.2763962F | chr5 | 147115232 | + | 9.24E−06 |
| cg20768326 | chr11 | 2086058 | − | 1.13E−05 |
| cg16876583 | chr14 | 59205523 | + | 2.49E−05 |
| cg03029993 | chr8 | 95538707 | − | 2.34E−06 |
| cg08909592 | chr20 | 60861650 | − | 6.98E−06 |
| cg14517144 | chr10 | 70098420 | + | 8.25E−07 |
| cg12946395 | chr1 | 180428472 | − | 1.38E−07 |
| cg27097660 | chr16 | 75241082 | + | 5.37E−06 |
| cg15408476 | chrX | 16733566 | − | 1.09E−05 |
| cg27374435 | chr6 | 79956068 | − | 5.15E−06 |
| cg01245965 | chr15 | 33278159 | − | 1.49E−05 |
| cg09669049 | chr14 | 52455814 | − | 7.52E−06 |
| cg09424138 | chr7 | 143700425 | + | 2.00E−05 |
| cg01372113 | chr6 | 88409436 | − | 2.57E−05 |
| cg02201720 | chrX | 148411481 | + | 2.31E−05 |
| cg16917903 | chr18 | 722818 | − | 6.05E−06 |
| cg14324167 | chr3 | 171851893 | − | 8.78E−06 |
| cg15840660 | chr1 | 153580069 | + | 1.39E−05 |
| cg06473615 | chr8 | 94248457 | + | 2.87E−06 |
| cg23507031 | chr11 | 122776303 | − | 2.84E−06 |
| cg14243426 | chr2 | 181018988 | − | 2.79E−06 |
| cg10296715 | chr5 | 45888975 | − | 5.04E−06 |
| cg12947436 | chr5 | 137010018 | + | 1.12E−05 |
| cg03283169 | chr12 | 78789229 | − | 1.55E−05 |
| cg04256065 | chr17 | 76102509 | + | 1.97E−05 |
| cg17187439 | chr2 | 205199965 | − | 4.58E−06 |
| cg10800837 | chr2 | 159238010 | + | 9.64E−06 |
| cg13380319 | chr7 | 78600265 | + | 1.14E−05 |
| ch.17.48901549F | chr17 | 51546550 | + | 1.03E−05 |
| cg01657331 | chr5 | 103185699 | + | 1.36E−05 |
| cg19430489 | chr12 | 75728104 | + | 1.16E−05 |
| cg23835971 | chr22 | 28629323 | − | 2.50E−05 |
| cg02240686 | chr2 | 37339522 | − | 9.46E−06 |
| cg08438775 | chr2 | 216447409 | + | 2.14E−06 |
| cg01909681 | chr1 | 42550741 | − | 4.97E−06 |
| cg26823782 | chr3 | 190611184 | + | 1.49E−05 |
| cg19981515 | chr21 | 17657567 | + | 2.25E−05 |
| cg05445721 | chr12 | 81016317 | + | 1.84E−05 |
| cg26737855 | chr12 | 18849720 | − | 1.33E−05 |
| cg17236066 | chr5 | 92276617 | + | 1.80E−05 |
| cg06100588 | chr12 | 116597819 | − | 5.20E−06 |
| cg00312746 | chr3 | 88237668 | − | 9.67E−06 |
| cg04976840 | chr4 | 118498212 | + | 7.69E−06 |
| cg21863114 | chr5 | 38530734 | + | 1.51E−05 |
| cg19907769 | chr2 | 20175418 | − | 2.58E−06 |
| cg13151527 | chr1 | 170248794 | − | 9.31E−06 |
| cg14665781 | chr10 | 88232080 | − | 2.53E−05 |
| cg10208282 | chr1 | 82319195 | + | 6.07E−06 |
| cg15501187 | chr8 | 92260185 | + | 1.27E−05 |
| cg00935819 | chr1 | 150672619 | − | 1.75E−06 |
| cg00186724 | chr3 | 193361784 | + | 4.32E−06 |
| cg26423824 | chr17 | 25898825 | − | 4.56E−06 |
| cg15160784 | chr1 | 117612890 | + | 2.12E−05 |
| cg08559317 | chr6 | 168952719 | + | 1.23E−05 |
| cg27135984 | chr8 | 14701972 | − | 1.34E−05 |
| cg08985840 | chr12 | 130733217 | + | 1.45E−05 |
| cg08500128 | chr12 | 10763670 | − | 9.27E−06 |
| cg24066561 | chr8 | 117148984 | − | 9.49E−06 |
| cg12594057 | chr10 | 27993940 | − | 2.46E−05 |
| cg17883444 | chr18 | 51886470 | + | 2.57E−05 |
| cg22719879 | chr6 | 129758529 | − | 2.56E−05 |
| cg03428028 | chr1 | 222990023 | + | 4.71E−06 |
| cg12636447 | chr8 | 56652350 | − | 1.83E−05 |
| cg02573743 | chr15 | 56120298 | − | 2.51E−05 |
| cg01317470 | chr2 | 48059543 | − | 2.12E−06 |
| cg09155944 | chr8 | 50734410 | − | 1.85E−05 |
| cg17392347 | chr2 | 163017274 | + | 1.58E−05 |
| cg15875417 | chr7 | 12249469 | − | 2.45E−05 |
| cg25925926 | chr11 | 111553032 | + | 9.85E−06 |
| cg06335220 | chr4 | 100132683 | + | 2.46E−05 |
| cg02921735 | chr5 | 119866103 | − | 2.55E−05 |
| cg22629015 | chr4 | 138433217 | + | 1.26E−05 |
| cg04247966 | chr3 | 16054139 | + | 5.54E−06 |
| cg13222022 | chr17 | 59017682 | + | 1.30E−05 |
| cg13667488 | chr6 | 48396235 | + | 2.54E−05 |
| cg13711885 | chr3 | 113821393 | − | 1.47E−05 |
| cg23301853 | chr14 | 97027326 | + | 1.80E−05 |
| cg20512247 | chr14 | 21030335 | − | 3.61E−06 |
| cg11330681 | chr5 | 55272791 | − | 5.59E−06 |
| cg07126637 | chr4 | 71248757 | − | 2.27E−05 |
| cg24004533 | chr5 | 122703229 | − | 1.21E−05 |
| cg09976979 | chr18 | 54605833 | + | 1.74E−05 |
| cg02781947 | chr4 | 132950908 | + | 1.21E−06 |
| cg21696677 | chr8 | 42788742 | − | 2.24E−05 |
| cg12287291 | chr11 | 122319811 | − | 2.10E−05 |
| cg27197209 | chr4 | 85499697 | − | 9.96E−06 |
| cg05558399 | chr11 | 104827790 | − | 5.71E−07 |
| cg10592563 | chr2 | 178974175 | + | 9.87E−06 |
| cg20791744 | chr3 | 24020542 | + | 3.61E−06 |
| cg12106037 | chr6 | 130476107 | − | 1.00E−05 |
| cg18940198 | chr3 | 107886300 | − | 5.16E−06 |
| cg12688244 | chr4 | 104067126 | − | 1.07E−06 |
| cg11324251 | chr3 | 170156458 | − | 1.52E−05 |
| cg06062784 | chr10 | 127414307 | + | 2.36E−05 |
| cg06774263 | chr11 | 85309753 | − | 9.70E−06 |
| cg04067446 | chr7 | 78821509 | − | 1.29E−05 |
| cg26695632 | chr18 | 27061916 | − | 1.54E−05 |
| cg24285847 | chr13 | 71187770 | − | 1.83E−05 |
| cg16796091 | chr5 | 140164954 | + | 2.39E−05 |
| cg17318391 | chr18 | 60626303 | + | 2.46E−05 |
| cg21441256 | chr12 | 93895324 | + | 7.79E−06 |
| cg03280604 | chr14 | 48184466 | + | 2.12E−05 |
| cg10931901 | chr14 | 101433314 | + | 9.31E−06 |
| cg11973514 | chr1 | 244004889 | − | 1.86E−05 |
| cg20894329 | chr11 | 106909409 | − | 2.47E−05 |
| cg18451035 | chr14 | 31398430 | − | 1.33E−05 |
| cg01036016 | chr5 | 36995804 | + | 2.34E−05 |
| cg25683803 | chr13 | 109507341 | − | 2.58E−06 |
| cg24482503 | chr7 | 143673129 | − | 2.52E−05 |
| cg16440806 | chr13 | 19601683 | − | 1.80E−05 |
| cg10413992 | chr8 | 39576637 | − | 4.22E−06 |
| cg21779576 | chr14 | 36302197 | + | 1.63E−05 |
| cg21040417 | chr2 | 50545418 | − | 1.21E−05 |
| cg27281482 | chr9 | 19799399 | − | 6.68E−06 |
| cg07833111 | chr12 | 128276059 | + | 8.60E−06 |
| cg22213475 | chr2 | 175245419 | − | 1.21E−05 |
| cg20849062 | chr2 | 131413611 | + | 2.23E−05 |
| cg04377041 | chr14 | 50190977 | − | 2.99E−06 |
| cg06305375 | chr16 | 2354020 | + | 9.28E−06 |
| cg25675096 | chr2 | 188361801 | − | 1.55E−05 |
| cg27618701 | chr2 | 24476586 | − | 2.44E−05 |
| cg20444539 | chr1 | 193604773 | + | 1.39E−05 |
| cg19891562 | chr8 | 131267301 | − | 1.09E−05 |
| cg11109498 | chr1 | 79099400 | + | 1.35E−06 |
| cg25904720 | chr3 | 27465120 | − | 1.13E−05 |
| cg27150644 | chr15 | 62283994 | − | 1.49E−05 |
| cg26746027 | chr4 | 94124976 | − | 1.42E−05 |
| cg07836549 | chr13 | 101823858 | + | 9.20E−06 |
| cg24481468 | chr8 | 118689069 | + | 9.13E−06 |
| cg16544246 | chr21 | 31990818 | − | 5.29E−06 |
| cg10493548 | chr6 | 66190665 | + | 2.58E−05 |
| cg03449978 | chr1 | 238891857 | − | 2.22E−05 |
| cg19538917 | chr5 | 177436178 | + | 9.94E−06 |
| cg21846877 | chr6 | 138323117 | − | 8.80E−06 |
| cg20271081 | chr4 | 158967544 | + | 2.68E−06 |
| cg03020114 | chr10 | 50714020 | − | 1.18E−05 |
| cg17471916 | chr21 | 32006771 | + | 2.33E−06 |
| cg00459613 | chr1 | 176050414 | − | 4.82E−06 |
| cg12136740 | chr20 | 5566935 | − | 1.60E−05 |
| cg26762723 | chr4 | 85725951 | − | 1.72E−05 |
| cg19434309 | chr1 | 236078188 | + | 1.37E−05 |
| cg03957278 | chr14 | 39536563 | − | 2.10E−05 |
| cg05331582 | chr7 | 87872522 | + | 6.95E−06 |
| cg17528325 | chr11 | 124094053 | + | 1.15E−05 |
| cg23675438 | chr21 | 38792235 | + | 1.06E−05 |
| cg25479848 | chr6 | 142490181 | + | 8.08E−06 |
| cg26442740 | chr12 | 55613318 | + | 1.87E−06 |
| cg05160248 | chr6 | 157284510 | + | 6.77E−06 |
| cg22753947 | chr2 | 210195918 | + | 2.03E−05 |
| cg07583848 | chr2 | 108779984 | + | 1.96E−05 |
| cg09750308 | chr8 | 89712356 | − | 1.31E−05 |
| cg01658829 | chr3 | 160129804 | + | 6.53E−06 |
| cg07345911 | chr6 | 145110334 | + | 2.03E−05 |
| cg07264682 | chr10 | 60082823 | − | 1.19E−05 |
| cg21365133 | chr21 | 31990349 | + | 2.37E−05 |
| cg12114129 | chr3 | 119776654 | − | 1.20E−05 |
| cg05130503 | chr5 | 92364521 | − | 8.35E−06 |
| cg11910279 | chr2 | 42865057 | + | 1.70E−05 |
| cg12821988 | chr6 | 49938799 | + | 1.65E−05 |
| cg18182039 | chr12 | 93165808 | + | 1.60E−05 |
| cg15622891 | chr6 | 163862445 | + | 2.57E−05 |
| cg11714341 | chr5 | 158605914 | − | 1.33E−05 |
| cg27097973 | chr13 | 24486258 | + | 1.79E−05 |
| cg00884574 | chr15 | 35811683 | − | 7.99E−06 |
| cg05434397 | chr4 | 166243335 | + | 1.95E−05 |
| cg04840732 | chr8 | 17156032 | − | 1.23E−05 |
| cg00748861 | chr19 | 46620011 | + | 2.13E−05 |
| cg13598642 | chr16 | 64473948 | + | 8.40E−06 |
| cg16131943 | chr10 | 26447082 | + | 9.79E−06 |
| cg19730706 | chr15 | 20877584 | − | 3.49E−06 |
| cg15597917 | chr12 | 81676362 | − | 1.79E−06 |
| cg22127335 | chr15 | 40188132 | − | 2.58E−05 |
| cg19789315 | chr4 | 33890657 | + | 1.82E−05 |
| cg25627364 | chr13 | 97050032 | + | 2.31E−05 |
| cg11195968 | chr5 | 144674171 | − | 1.56E−05 |
| cg25128243 | chr1 | 247767614 | − | 6.48E−06 |
| cg04582186 | chr8 | 9954880 | + | 1.25E−05 |
| cg19139370 | chr5 | 77347424 | − | 2.39E−05 |
| cg26499430 | chr8 | 29882170 | − | 1.32E−05 |
| cg16165258 | chr12 | 76927207 | − | 2.07E−05 |
| ch.20.53118117F | chr20 | 53684710 | + | 2.13E−05 |
| cg27608224 | chr6 | 72922399 | + | 2.50E−06 |
| cg14396780 | chr4 | 186168717 | + | 2.22E−05 |
| cg23722232 | chr5 | 94856457 | − | 4.26E−06 |
| cg11563784 | chr2 | 139132471 | − | 3.22E−06 |
| cg02540423 | chr2 | 236711871 | + | 4.16E−06 |
| cg17894592 | chr4 | 139052047 | + | 1.31E−05 |
| cg22375856 | chr3 | 17301676 | − | 1.56E−05 |
| cg23199018 | chrX | 36549203 | − | 4.79E−06 |
| cg20269160 | chr14 | 101428040 | + | 1.74E−05 |
| cg07381572 | chr3 | 170630736 | + | 5.19E−06 |
| cg16709010 | chr1 | 54983173 | + | 1.65E−05 |
| cg04958800 | chr7 | 126884515 | + | 7.50E−06 |
| cg07029980 | chr7 | 104640734 | − | 1.92E−05 |
| cg06187402 | chr3 | 24844156 | + | 1.14E−05 |
| cg27493500 | chr11 | 5246823 | + | 1.89E−05 |
| cg07454944 | chr7 | 121726304 | − | 1.58E−05 |
| cg03356461 | chr1 | 196003367 | − | 1.13E−06 |
| cg11778270 | chr12 | 48058688 | − | 9.25E−06 |
| cg15353890 | chr12 | 124140125 | − | 3.66E−06 |
| cg02858642 | chr1 | 202312902 | + | 2.06E−06 |
| cg08630891 | chr7 | 32557733 | + | 1.05E−05 |
| cg24076381 | chr14 | 101383762 | − | 2.38E−05 |
| cg21531518 | chr2 | 187424886 | − | 2.54E−06 |
| cg08772528 | chr9 | 77571744 | − | 1.60E−05 |
| cg12497171 | chr19 | 44763334 | − | 1.22E−05 |
| cg26816929 | chr17 | 67352774 | − | 8.53E−06 |
| cg08189964 | chr4 | 48104648 | − | 5.80E−06 |
| cg13433782 | chr4 | 43460502 | − | 4.59E−07 |
| cg07008779 | chr1 | 153072131 | − | 1.57E−05 |
| cg02164185 | chr8 | 63940615 | + | 1.54E−05 |
| cg00375905 | chr18 | 19396187 | + | 5.46E−06 |
| cg18251430 | chr15 | 88017639 | + | 1.86E−05 |
| cg02877451 | chr18 | 27359367 | + | 2.36E−05 |
| cg27156917 | chr12 | 18435578 | + | 4.29E−06 |
| cg20141733 | chr1 | 73425038 | + | 3.62E−06 |
| cg05178153 | chr5 | 41854892 | + | 2.41E−05 |
| cg25151936 | chr14 | 70939596 | + | 1.27E−05 |
| cg25792284 | chr3 | 102474211 | + | 6.56E−06 |
| cg25958487 | chr10 | 88230819 | − | 1.31E−06 |
| cg06304894 | chr5 | 63870294 | − | 9.76E−06 |
| cg15202115 | chr8 | 79305876 | − | 1.95E−05 |
| cg06865309 | chr5 | 161479730 | + | 8.59E−06 |
| cg20345740 | chr11 | 6914091 | − | 7.11E−06 |
| cg27284962 | chr8 | 92032409 | − | 3.02E−06 |
| cg24562864 | chr3 | 78695476 | − | 1.84E−05 |
| cg10874403 | chr3 | 87325835 | − | 1.79E−05 |
| cg20842670 | chr4 | 176685457 | − | 5.17E−06 |
| cg03939903 | chr2 | 180662010 | − | 4.76E−06 |
| cg21154181 | chr3 | 169693090 | + | 2.51E−05 |
| cg06479216 | chr9 | 21218462 | + | 2.24E−05 |
| cg02806251 | chr2 | 148022810 | + | 1.74E−05 |
| cg23952322 | chr12 | 87717355 | + | 3.75E−06 |
| cg14797608 | chr7 | 138609880 | + | 1.95E−05 |
| cg21200229 | chr7 | 136588030 | − | 2.30E−05 |
| cg09787381 | chr7 | 134643566 | + | 1.20E−05 |
| cg06558137 | chr6 | 46425781 | + | 8.84E−06 |
| cg10413297 | chr22 | 32767648 | − | 1.38E−05 |
| cg00961823 | chr5 | 86921431 | + | 2.54E−06 |
| cg24125651 | chr1 | 109456980 | + | 3.42E−06 |
| cg09461630 | chr12 | 41156128 | − | 3.36E−06 |
| cg22334320 | chr12 | 38718698 | + | 4.10E−07 |
| cg19515919 | chr6 | 79693583 | − | 2.08E−05 |
| cg10083765 | chr6 | 108713075 | + | 4.25E−06 |
| cg01651751 | chr2 | 203329535 | + | 2.39E−05 |
| cg27171564 | chr12 | 79641612 | + | 2.33E−05 |
| cg24410305 | chr11 | 28899715 | + | 1.58E−05 |
| cg24916165 | chr2 | 47595507 | − | 1.72E−05 |
| cg05590522 | chr1 | 98675035 | + | 7.52E−06 |
| cg25283172 | chr2 | 29038696 | + | 2.21E−05 |
| cg03267512 | chr8 | 21796065 | − | 1.67E−05 |
| cg08965435 | chr11 | 129034338 | − | 2.39E−05 |
| cg04583116 | chr7 | 119184686 | − | 1.05E−05 |
| cg06532348 | chr1 | 87177981 | + | 6.54E−06 |
| cg05667581 | chr21 | 11105235 | + | 1.82E−05 |
| cg16213835 | chr4 | 144464780 | + | 7.70E−06 |
| cg04584894 | chr2 | 103305353 | + | 7.82E−06 |
| cg01296976 | chr5 | 65454376 | + | 9.88E−07 |
| cg25016974 | chr1 | 227500404 | − | 1.44E−05 |
| cg26905126 | chr16 | 47362879 | − | 2.31E−05 |
| cg25528758 | chr10 | 60994548 | + | 1.66E−05 |
| cg22179430 | chr3 | 140675446 | + | 6.93E−06 |
| cg10390473 | chr4 | 164775263 | − | 1.78E−05 |
| cg27351449 | chr6 | 135606436 | − | 1.15E−05 |
| cg20616246 | chr1 | 180010285 | + | 4.38E−06 |
| cg13259312 | chr4 | 149478932 | + | 2.44E−05 |
| cg06189459 | chr11 | 55737423 | − | 1.45E−05 |
| cg08949726 | chr2 | 178002466 | − | 1.17E−05 |
| cg01780928 | chr20 | 8186480 | + | 1.51E−05 |
| cg20294640 | chr3 | 150332279 | + | 9.11E−06 |
| cg14160443 | chr19 | 12216505 | + | 4.16E−06 |
| cg21920959 | chr12 | 68689164 | − | 2.02E−05 |
| cg20395359 | chr4 | 162421388 | − | 1.25E−05 |
| cg12719408 | chr10 | 22680067 | + | 2.36E−05 |
| cg00518770 | chr2 | 95521431 | − | 8.85E−06 |
| cg23547617 | chr10 | 71036794 | + | 1.23E−05 |
| cg02162815 | chr14 | 89305821 | + | 1.78E−05 |
| cg11374446 | chr13 | 106126915 | + | 6.43E−06 |
| cg03096378 | chr11 | 108377279 | − | 1.16E−05 |
| cg07168102 | chr8 | 97006078 | + | 1.18E−05 |
| cg02662654 | chr17 | 14038796 | + | 1.26E−05 |
| cg04036977 | chr1 | 241731922 | − | 2.09E−05 |
| cg14183389 | chr4 | 132194300 | − | 1.12E−05 |
| cg17051058 | chr8 | 101196190 | + | 1.37E−05 |
| cg01137698 | chr18 | 28926189 | − | 8.92E−06 |
| cg09688422 | chr11 | 105959824 | + | 2.06E−05 |
| cg16646004 | chr6 | 142540447 | + | 9.30E−06 |
| cg04695653 | chr3 | 108029010 | − | 7.46E−06 |
| cg22854591 | chr10 | 65220901 | − | 2.24E−05 |
| cg17437621 | chr6 | 99848161 | − | 9.84E−06 |
| cg17133439 | chr5 | 107700623 | − | 7.94E−06 |
| cg02743284 | chr2 | 72833370 | − | 8.67E−07 |
| cg26477387 | chr6 | 87973166 | + | 2.55E−05 |
| cg11361926 | chr4 | 132269562 | + | 1.41E−05 |
| cg26526113 | chr14 | 35180593 | − | 5.17E−06 |
| cg22664874 | chr4 | 71599063 | + | 1.11E−05 |
| cg20070619 | chr10 | 34626313 | − | 2.08E−05 |
| cg14565394 | chr2 | 69561027 | − | 9.07E−07 |
| cg22872634 | chr1 | 100462710 | + | 1.92E−06 |
| cg23307527 | chr1 | 58996322 | − | 2.37E−05 |
| cg17359407 | chr14 | 38264062 | + | 1.18E−06 |
| cg09736391 | chr13 | 29049271 | − | 1.02E−05 |
| cg07563429 | chr1 | 73184838 | + | 8.45E−06 |
| cg03673191 | chr21 | 28952919 | − | 4.22E−06 |
| cg07780082 | chr18 | 72474107 | − | 2.49E−05 |
| cg18864334 | chr6 | 163358032 | + | 5.83E−06 |
| cg24673772 | chr5 | 13577191 | − | 6.72E−06 |
| cg01654242 | chr8 | 101155972 | − | 2.05E−06 |
| cg25515937 | chr1 | 79845965 | + | 1.11E−05 |
| cg12765107 | chr7 | 90315680 | + | 2.19E−05 |
| cg16664455 | chr4 | 94125068 | + | 2.33E−05 |
| cg24649139 | chr10 | 25617001 | + | 1.15E−05 |
| cg24806210 | chr18 | 70819023 | − | 1.77E−05 |
| cg02979850 | chr11 | 55682603 | + | 2.49E−05 |
| cg08475898 | chr6 | 32320708 | + | 7.02E−06 |
| cg08955884 | chr11 | 7109460 | − | 2.12E−05 |
| cg09030852 | chr4 | 170629768 | + | 8.00E−06 |
| cg13698937 | chr4 | 159589048 | − | 9.73E−06 |
| cg13495204 | chr4 | 74274452 | + | 1.06E−06 |
| cg10827090 | chr21 | 17252050 | + | 1.75E−05 |
| cg02329358 | chr7 | 142894595 | + | 8.60E−06 |
| cg12457604 | chr12 | 122826983 | − | 2.48E−05 |
| cg24279075 | chr13 | 87744249 | + | 1.79E−05 |
| cg19169186 | chrX | 31984302 | − | 1.42E−05 |
| cg13616190 | chr8 | 88041484 | + | 3.08E−06 |
| cg21911195 | chr2 | 203328473 | + | 2.13E−05 |
| cg22783999 | chr6 | 29578144 | − | 1.42E−05 |
| cg10305829 | chr4 | 176686709 | − | 3.07E−06 |
| cg09628898 | chr5 | 88330429 | − | 6.78E−07 |
| cg17538137 | chr10 | 4468714 | + | 2.46E−05 |
| cg03855973 | chr6 | 76315197 | + | 1.96E−05 |
| cg10347226 | chr16 | 48692412 | − | 1.68E−05 |
| cg12442012 | chr18 | 66366609 | − | 4.10E−06 |
| cg15816896 | chr3 | 164403954 | − | 2.00E−05 |
| cg03594546 | chr2 | 170916444 | + | 1.22E−05 |
| cg20838101 | chr9 | 37820310 | + | 3.75E−06 |
| cg04965365 | chr9 | 108487768 | + | 4.95E−06 |
| cg19920029 | chr14 | 47411755 | − | 1.51E−05 |
| cg09451747 | chr4 | 143542302 | − | 1.45E−05 |
| cg15180379 | chr6 | 100891601 | + | 2.03E−05 |
| cg05128619 | chr5 | 27987467 | − | 2.27E−05 |
| cg08580056 | chr13 | 60516055 | − | 4.95E−06 |
| cg14935315 | chr7 | 56123769 | + | 2.98E−06 |
| cg27446385 | chr14 | 38264058 | + | 2.41E−05 |
| cg22654478 | chr14 | 45565167 | + | 1.68E−05 |
| cg08399134 | chr6 | 126344877 | − | 2.52E−05 |
| cg16602117 | chr8 | 83728972 | + | 1.40E−05 |
| cg17758905 | chr1 | 48648592 | + | 1.03E−06 |
| cg26415605 | chr13 | 52704602 | − | 2.13E−05 |
| cg07625992 | chr10 | 61007196 | − | 2.30E−05 |
| cg02315483 | chr19 | 44487129 | + | 1.16E−05 |
| cg05284211 | chr7 | 41400299 | − | 2.27E−05 |
| cg18205149 | chr4 | 103311355 | − | 1.76E−05 |
| cg18228017 | chr17 | 57991443 | + | 2.08E−05 |
| cg14302778 | chr12 | 42078465 | − | 1.01E−05 |
| cg08612570 | chr7 | 27868767 | + | 1.03E−06 |
| cg14198080 | chr7 | 112096328 | + | 1.63E−05 |
| cg01695412 | chr3 | 196243555 | + | 2.21E−06 |
| cg20489932 | chr10 | 93220698 | + | 6.03E−06 |
| cg09333367 | chr17 | 18738729 | + | 2.01E−05 |
| cg25155679 | chr13 | 67479146 | − | 2.39E−05 |
| cg20108671 | chr15 | 77154813 | − | 1.52E−05 |
| cg23318724 | chr15 | 44760879 | + | 2.29E−05 |
| cg12584735 | chr3 | 89350961 | + | 2.55E−05 |
| cg07987196 | chr8 | 101727818 | − | 1.58E−05 |
| cg23543824 | chr4 | 70384684 | + | 1.55E−05 |
| cg02619087 | chr12 | 103838906 | − | 4.81E−06 |
| cg01860944 | chr13 | 61455634 | + | 8.18E−06 |
| cg24914842 | chr5 | 88039920 | − | 1.74E−05 |

With reference to Table 4, the following 13 sites were hypermethylated: cg02858642, ch.20.53118117F, cg20768326, ch.17.48901549F, cg15202115, cg01372113, cg10296715, cg19430489, cg03356461, cg09669049, cg21846877, ch.5.2763962F, and cg07029980. The remaining sites listed in Table 4 were hypomethylated.

### Detection of Bacterial Markers

The human microbiome also presents a potential source of novel biomarkers for detection of endometriosis. The microbiome is the collection of microorganisms in the body that exists in a mutualistic relationship with the host. The microbial metagenome of cervicovaginal and menstrual fluid was analyzed to understand the bacterial diversity present in endometriosis compared to healthy controls (both truly health and suspected unhealthy—Table 1A). Within the analyzed population there were 5 patients with polycystic ovarian syndrome, 19 with endometriosis (both pre- and post-surgery collected tampons), and 5 healthy and 50 “suspected unhealthy” individuals. 16s microbial sequencing was performed, where a region of the ribosomal RNA genomic code was amplified and sequenced, enabling species-level resolution of bacterial composition. This information was used to compare the relative abundance of bacterial species between healthy (broken up into truly healthy and suspected unhealthy—Table 1A), polycystic ovarian syndrome, and endometriosis. The diversity present within each sample (alpha diversity) was then examined as well as the diversity present between samples in the same cohort (beta diversity). The Shannon Diversity Index was used, which takes into account the abundance of each bacterial species, as well as how evenly that species is represented within the sample or population. An increased diversity of bacterial species was found among patients with endometriosis compared to healthy patients (FIGS. 4A and 4B). This study was able to identify specific bacterial species that were associated with endometriosis, most notably, Propionibacterium acnes, which is present at 15-fold higher levels in the endometriosis patients than in healthy individuals. P. acnes produces high levels of prostaglandin-like substances and porphyrin, both of which have been implicated in inflammation and dysmenorrhea.

Further, by comparing the abundance of bacteria in healthy cohort 1 of menstrual samples to cervicovaginal samples, a large number of bacterial genus are observed to have higher abundance in menstrual fluid than cervicovaginal (FIG. 4C). This is unique to this cohort, creating a unique bacterial signature. Abundance was also compared in healthy cohort three, giving a much more exhaustive list of bacteria overrepresented in menstrual fluid. This increase in number of bacterial genuses in healthy cohort three shows a correlation of number of overabundant species to degree of “healthiness” in patient cohorts (FIG. 4D). By looking at just menstrual blood, many bacterial genuses are observed to be present and higher abundance or lower abundance than in healthy cohort 1 (FIG. 4E).

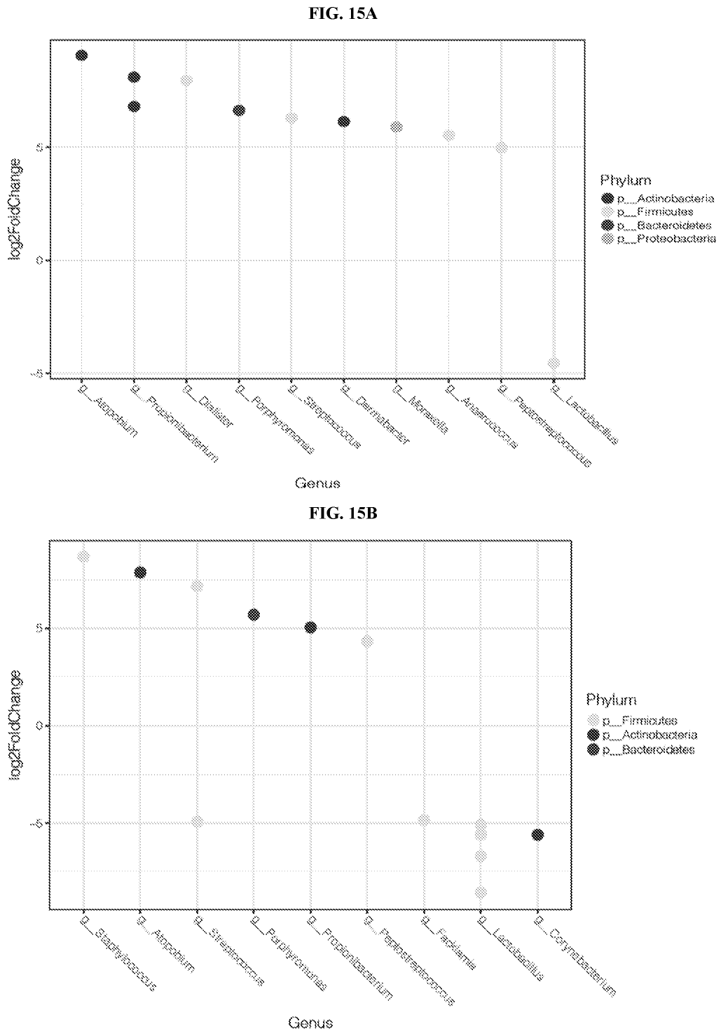

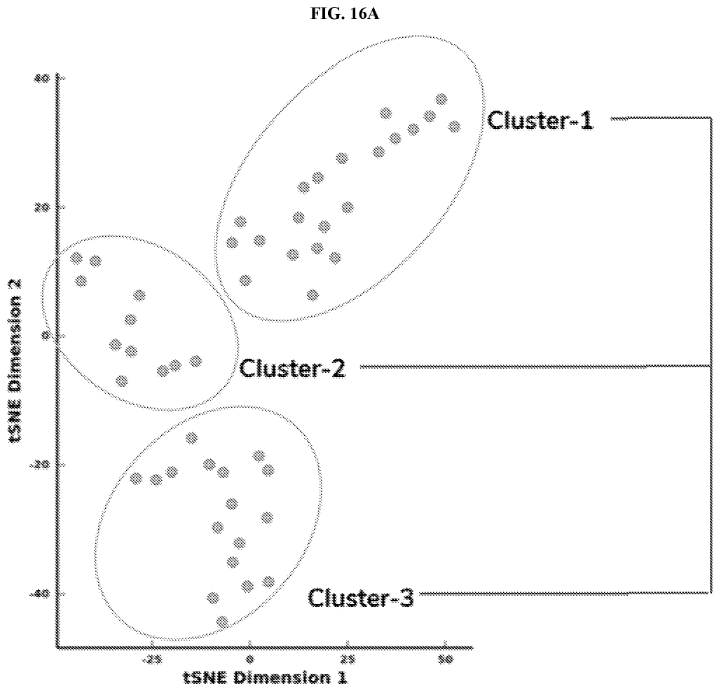

Further, when the menstrual fluid samples are compared between pre-surgery endometriosis patients to health controls, there is another signature of differentially present bacterial genuses unique to pre-surgery patients, as depicted in FIG. 15A. Post-surgery, the bacterial abundance in menstrual fluid between endometriosis patients and healthy patients is depicted in FIG. 15B.

### Example 4

### Differential Methylation Patterns Between Menstrual Fluid and Whole Blood

RAW IDAT files were provided from IlluminaHumanMethylationEPIC array. 311 sample files were available: 50 menstrual blood samples, 26 whole blood samples, and 253 other samples. Data processing was performed using the minfiR package (Bioconductor).

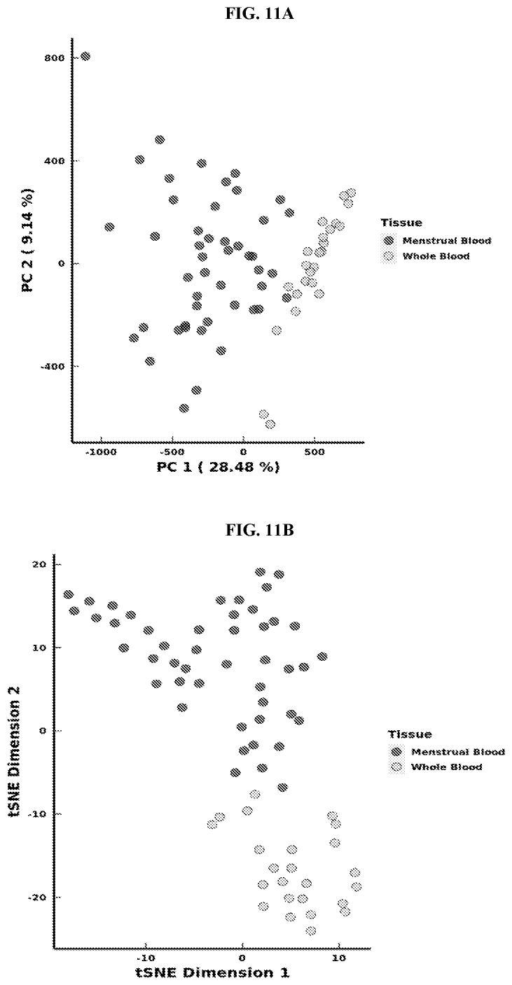

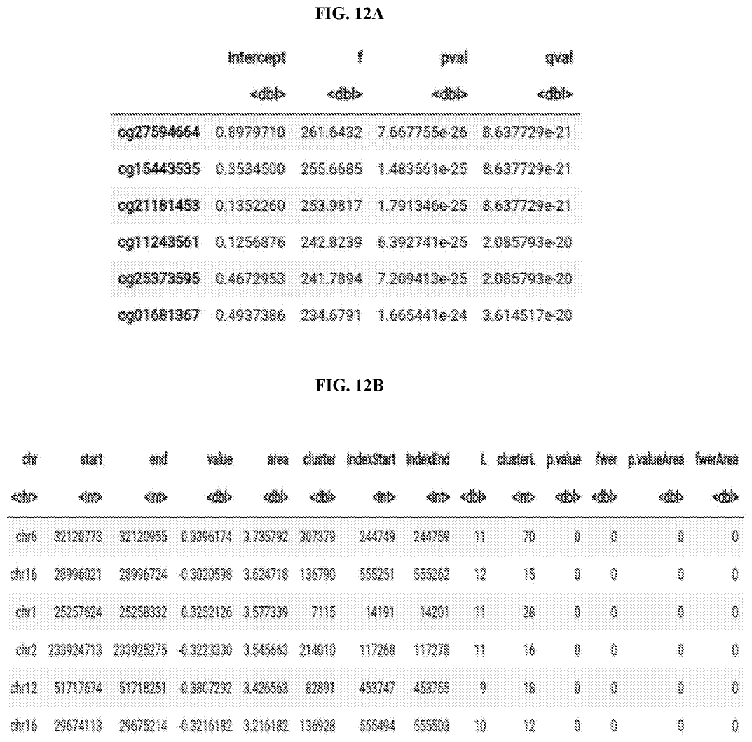

When the methylation patterns of whole blood and menstrual blood were analyzed by principle component analysis and tSNE dimensionality by tissue type, they showed clear differentiation by tissue type, as depicted in FIG. 11A-11B. FIG. 12A displays differentially methylated CpG positions when comparing whole blood and menstrual blood. FIG. 12B displays differentially methylated regions between whole blood and menstrual blood.

### Example 5

### Use of a Menstrualome Fingerprint on Patient Data

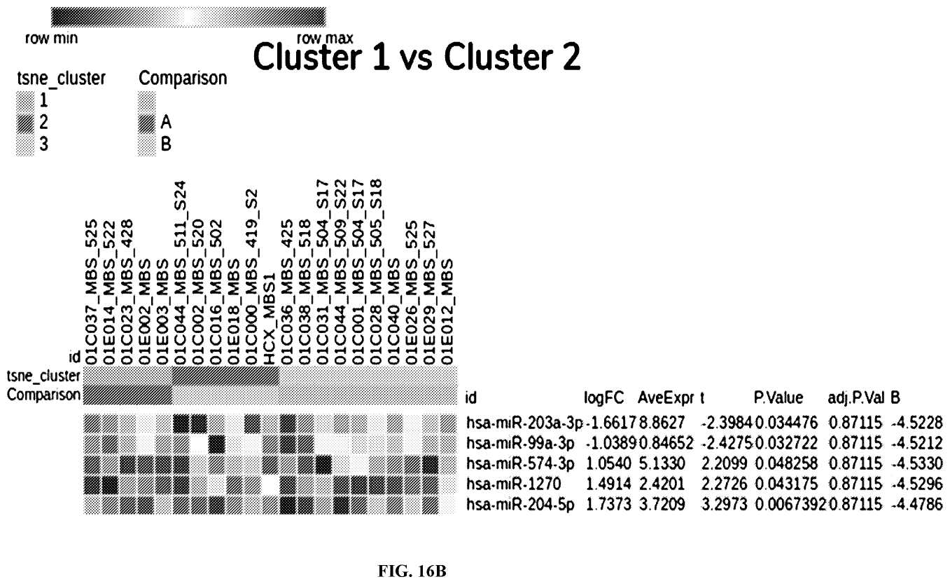

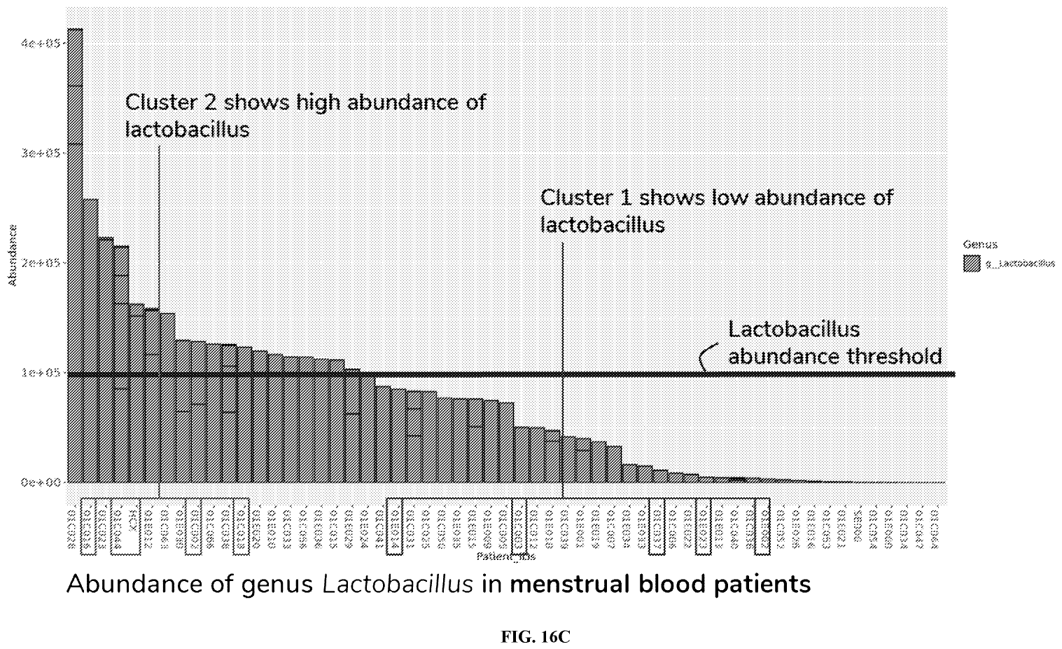

For many patients there may not be phenotypic data to group patients, or there may be an undiagnosed health condition that could hamper analysis if data is only analyzed by phenotypic and clinical parameters. Therefore, it is important to have a data-driven approach without a priori knowledge of patient data. In FIG. 16A-FIG. 16B, we can see that the methylation data for menstrual fluid among our patient cohort has 3 distinct clusters. These clusters can then be used to begin grouping patients into cohorts for additional genomic analysis. In this example, methylation clusters were used to set patient cohorts for differential expression analysis of miRNA expression collected on the same patients. This then produced a list of significantly dysregulated miRNAs for each cluster.

The patients from Clusters 1 and 2 from the methylation data and were compared to the abundance of lactobacillus within the menstrual sample (decreased lactobacillus is generally indicative of some unhealthy state). As seen in FIG. 16C, cluster 1 shows low abundance for lactobacillus, while cluster 2 shows high abundance. From this a general healthy vs unhealthy state can be imputed for each of these clusters. Cluster 1 would denote unhealthy, while cluster 2 would denote healthy.

Biological relevance can be determined by taking the miRNA targets and looking at the gene expression (RNA-seq) data to see if an expression change occurs in these genes that shows that the correlation is real. Average expression values are presented for cluster 1 and cluster two for each of the genes that are targeted in the 5 miRNAs dysregulated between clusters 1 and clusters 2 in Table 5. The log 2 fold change between clusters 1 and 2 are presented to demonstrate the expression changes between clusters.

TABLE 5
Expression change by cluster

| | Average | |
|---|---|---|
| | Expression | Log2 fold change of |
| cluster | value | cluster 1 vs. cluster 2 |
| CYP26A1 cluster 1 | 0.22606087 | 1.286420439 |
| CYP26A1 cluster 2 | 0.09267749 | |
| BPIFB1 cluster 1 | 0.30686597 | 0.343109732 |
| BPIFB1 cluster 2 | 0.24191478 | |
| SCGB2A2 cluster 1 | 1.22321014 | −0.711181248 |
| SCGB2A2 cluster 2 | 2.00257117 | |
| CDC42BPA cluster 1 | 1.03676009 | 0.293218741 |
| CDC42BPA cluster 2 | 0.84607845 | |

Cluster 1 is enriched for confirmed endometriosis patients. The miRNAs differentially regulated based on methylation clusters (miR-1270, miR-204-5p, miR-574-3p, miR-203a-3p, and miR-99a-3p) show overexpression of 5 key microRNAs involved in regulation of CYP26A1, BPIFB1, SCGB2A2, CDC42BPA. Of these genes, CYP26A1 is a retinoic acid regulator that is progesterone dependent and highly dysregulated in endometriosis; BPIFB1 is a molecular signature of eutopic endometrium in patients with endometriosis and is significantly downregulated in patients; SCGB2A2 is significantly downregulated in patients with endometriosis; and CDC42BPA, a key cell cycle regulator in the menstrual cycle, and is significantly dysregulated in patients with endometriosis. These miRNAs show biological relevance when compared to RNA-seq data. Likewise, endometriosis has been associated with lower abundance of lactobacillus, similar to cluster 1 patients.

### REFERENCES

1 Laudanski, P., Charkiewicz, R., Kuzmicki, M. et al. MicroRNAs expression profiling of eutopic proliferative endometrium in women with ovarian endometriosis. Reprod Biol Endocrinol 11, 78 (2013). https://doi.org/10.1186/1477-7827-11-78
2 Wright K R, Mitchell B, Santanam N. Redox regulation of microRNAs in endometriosis-associated pain. Redox Biol. 2017; 12:956-966. doi:10.1016/j.redox.2017.04.037
3 Filigheddu N, Gregnanin I, Porporato P E, et al. Differential expression of microRNAs between eutopic and ectopic endometrium in ovarian endometriosis. J Biomed Biotechnol. 2010; 2010:369549. doi:10.1155/2010/369549
4 Hawkins S M, Creighton C J, Han D Y, et al. Functional microRNA involved in endometriosis. Mol Endocrinol. 2011; 25(5):821-832. doi:10.1210/me.2010-0371
5 Nisenblat V, Circulating miRNAs in endometriosis, The Robinson Institute, 2013; https://digital.library.adelaide.edu.au/dspace/bitstream/2440/91226/5/01front.pdf
6 Vanhie A, O D, Peterse D, et al. Plasma miRNAs as biomarkers for endometriosis. Hum Reprod. 2019;34(9):1650-1660. doi:10.1093/humrep/dez116
7 Cosar E, Mamillapalli R, Ersoy G S, Cho S Y, Seifer B, Taylor H S, Serum microRNAs as diagnostic markers of endometriosis: a comprehensive array-based analysis, FertSterility. 2016.DOI:https://doi.org/10.1016/j.fertnstert.2016.04.013
8 WO2015148919A3; Circulating micrornas as biomarkers for endometriosis
9 Ohlsson Teague E M, Van der Hoek K H, Van der Hoek M B, et al. MicroRNA-regulated pathways associated with endometriosis. Mol Endocrinol. 2009; 23(2):265-275. doi:10.1210/me.2008-0387
10 Zhuo, Z.; Wang, C.; Li, G.; Yu, H. Plasma MicroRNAs Can be a Potential Diagnostic Biomarker for Endometriosis. Preprints 2019, 2019070108 (doi:10.20944/preprints201907.0108.v1).
11 Zhao L, Gu C, Ye M, et al. Integration analysis of microRNA and mRNA paired expression profiling identifies deregulated microRNA-transcription factor-gene regulatory networks in ovarian endometriosis. Reprod Biol Endocrinol. 2018;16(1):4. Published 2018 January 22. doi:10.1186/s12958-017-0319-5
12 Suryawanshi, S and Vlad, A M. Lin, H M et al. Plasma MicroRNAs as Novel Biomarkers for Endometriosis and Endometriosis-Associated Ovarian Cancer, American Association for Cancer Research, 2013. https://doi.org/10.1158/1078-0432.CCR-12-2726
13 Zhou C F, Liu M J, Wang W, et al. miR-205-5p inhibits human endometriosis progression by targeting ANGPT2 in endometrial stromal cells [published correction appears in Stem Cell Res Ther. 2020 May 20; 11(1):188]. Stem Cell Res Ther. 2019; 10(1):287. Published 2019 Sep. 23. doi:10.1186/s13287-019-1388-5
14 Kim, M. K., Lee, S. K., Park, J. et al. Ginsenoside Rg3 Decreases Fibrotic and Invasive Nature of Endometriosis by Modulating miRNA-27b: In Vitro and In Vivo Studies. Sci Rep 7, 17670 (2017). https://doi.org/10.1038/s41598-017-17956-0

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for preparation of a menstrualome fingerprint, comprising:
(a) obtaining a first sample and a second sample from a subject, wherein the first sample and the second sample comprise cervicovaginal or menstrual fluid collected onto a first and second absorbent sample collector;
(b) eluting the first sample and the second sample separately from the first and second sample collector into an aqueous buffer;
(c) separating a biological material from each of the first sample and the second sample; and
(d) constructing a sample menstrualome fingerprint, wherein the sample menstrualome fingerprint comprises the differential of the level and/or presence of a plurality of menstrualome biomarkers in the biological material from the first sample and the second sample.

2. The method of claim 1, wherein the biological material comprises one or more biological materials selected from the group consisting of a RNA, a DNA, a methylated nucleic acid, a miRNA, a protein, a protein-nucleic acid complex, a microorganism, and a mammalian cell type.

3. The method of claim 1, wherein constructing the sample menstrualome fingerprint in (d) comprises assaying the extracted biological material from the first sample and the second sample to identify a plurality of biomarkers.

4. The method of claim 1, wherein the plurality of menstrualome biomarkers comprise biomarkers that display differential presence or level in cervicovaginal or menstrual fluid between two or more health states.

5. The method of claim 1, wherein the plurality of menstrualome biomarkers comprise biomarkers that display differential presence or level in cervicovaginal or menstrual fluid as compared to peripheral blood, cervicovaginal tissue, or a longitudinal menstrual sample.

6. The method of claim 1, further comprising (e) comparing the sample menstrualome fingerprint to a reference menstrualome fingerprint.

7. The method of claim 6, wherein the reference menstrualome fingerprint comprises a threshold level or presence of the plurality of menstrualome biomarkers that are associated with a health state.

8. The method of claim 1, wherein the first sample and second sample comprise biological material collected at a different time points from the subject.

9. The method of claim 8, wherein the time points are separated by a time period between about 15 minutes and about 30 days, about 60 days, or about 90 days.

10. The method of claim 8, wherein the time points comprise different days within a menstrual cycle of the subject.

11. The method of claim 8, wherein the time points are within a single menstrual cycle.

12. The method of claim 8, wherein the time points comprise days in separate menstrual cycles.

13. The method of claim 8, wherein the time points are during one or more days of menstruation of the subject.

14. The method of claim 8, wherein one time point is during menstruation of the subject and one time point is not during menstruation of the subject.

15. The method of claim 1, wherein the sample collector is an intravaginal sample collector.

16. The method of claim 1, wherein the sample collector preserves a biological material in an intact state.

17. The method of claim 1, wherein the sample collector is capable of absorbing at least 3 ml of fluid.

18. The method of claim 1, wherein the sample collector is placed into a buffer subsequent to collecting the sample.

19. The method of claim 1, wherein the biological material is DNA and plurality of menstrualome biomarkers comprises methylation status of a plurality of loci.

20. The method of claim 1, wherein the biological material is RNA and plurality of menstrualome biomarkers comprises expression level of a plurality of genes.

21. The method of claim 1, wherein the biological material is RNA and a plurality of menstrualome biomarkers comprises the presence and/or level of a plurality of miRNAs.

22. The method of claim 1, wherein the biological material is cells and plurality of menstrualome biomarkers measures the presence and/or amount of one or more cell types.

23. The method of claim 1, wherein the biological material is DNA and plurality of menstrualome biomarkers measures the presence and/or level of one or more microorganisms.

24. The method of claim 1, wherein the biological material is DNA and plurality of menstrualome biomarkers measures the diversity of microorganisms.

25. The method of claim 4, wherein the two or more health states comprise before and after a medical treatment.

26. The method of claim 7, wherein the health state comprises a health state before surgery.

27. The method of claim 7, wherein the reference state comprises a health state after surgery.

28. The method of claim 7, wherein the health state comprises a menstrual disorder.

29. The method of claim 28, wherein the health state comprises endometriosis.

30. The method of claim 7, wherein the health state comprises a healthy patient.

31. The method of claim 7, wherein the health reference menstrualome fingerprint comprises a principle component analysis, a t-Distributed Stochastic Neighbor Embedding, a heat map, a diversity index, or a combination thereof.

32. A method for preparation of a menstrualome fingerprint, comprising:
(a) obtaining a first sample and a second sample from a subject, wherein the first sample and the second sample comprise cervicovaginal or menstrual fluid collected onto a first and second absorbent sample collector;
(b) eluting the first sample and the second sample separately from the first and second sample collector into an aqueous buffer;
(c) separating a biological material from each of the first sample and the second sample; and
(d) constructing a sample menstrualome fingerprint, wherein the sample menstrualome fingerprint comprises the differential of the level and/or presence of a plurality of menstrualome biomarkers in the biological material from the first sample and/or the second sample as compared to a reference menstrualome fingerprint.

33. The method of claim 32, wherein the biological material comprises one or more biological materials selected from the group consisting of a RNA, a DNA, a methylated nucleic acid, a miRNA, a protein, a protein-nucleic acid complex, a microorganism, and a mammalian cell type.

34. The method of claim 32, wherein constructing the sample menstrualome fingerprint in (d) comprises assaying the extracted biological material from the first sample and the second sample to identify a plurality of biomarkers.

35. The method of claim 32, wherein the plurality of menstrualome biomarkers comprise biomarkers that display differential presence or level in cervicovaginal or menstrual fluid between two or more health states.

36. The method of claim 32, wherein the plurality of menstrualome biomarkers comprise biomarkers that display differential presence or level in cervicovaginal or menstrual fluid as compared to peripheral blood, cervicovaginal tissue, or a longitudinal menstrual sample.

37. The method of claim 32, wherein the reference menstrualome fingerprint comprises a threshold level or presence of the plurality of menstrualome biomarkers that are associated with a health state.

38. The method of claim 32, wherein the first sample and second sample comprise biological material collected at a different time points from the subject.

39. The method of claim 38, wherein the time points are separated by a time period between about 15 minutes and about 30 days, about 60 days, or about 90 days.

40. The method of claim 38, wherein the time points comprise different days within a menstrual cycle of the subject.

41. The method of claim 38, wherein the time points are within a single menstrual cycle.

42. The method of claim 38, wherein the time points comprise days in separate menstrual cycles.

43. The method of claim 38, wherein the time points are during one or more days of menstruation of the subject.

44. The method of claim 38, wherein one time point is during menstruation of the subject and one time point is not during menstruation of the subject.

45. The method of claim 32, wherein the sample collector is an intravaginal sample collector.

46. The method of claim 32, wherein the sample collector preserves a biological material in an intact state.

47. The method of claim 32, wherein the sample collector is capable of absorbing at least 3 ml of fluid.

48. The method of claim 32, wherein the sample collector is placed into a buffer subsequent to collecting the sample.

49. The method of claim 32, wherein the biological material is DNA and plurality of menstrualome biomarkers comprises methylation status of a plurality of loci.

50. The method of claim 32, wherein the biological material is RNA and plurality of menstrualome biomarkers comprises expression level of a plurality of genes.

51. The method of claim 32, wherein the biological material is RNA and plurality of menstrualome biomarkers comprises the presence and/or level of a plurality of miRNAs.

52. The method of claim 32, wherein the biological material is cells and plurality of menstrualome biomarkers measures the presence and/or amount of one or more cell types.

53. The method of claim 32, wherein the biological material is DNA and plurality of menstrualome biomarkers measures the presence and/or level of one or more microorganisms.

54. The method of claim 32, wherein the biological material is DNA and plurality of menstrualome biomarkers measures the diversity of microorganisms.

55. The method of claim 37, wherein the two or more health states comprise before and after a medical treatment.

56. The method of claim 37, wherein the health state comprises a health state before surgery.

57. The method of claim 32, wherein the reference state comprises a health state after surgery.

58. The method of claim 37, wherein the health state comprises a menstrual disorder.

59. The method of claim 58, wherein the health state comprises endometriosis.

60. The method of claim 37, wherein the health state comprises a healthy patient.

61. The method of claim 37, wherein the health reference menstrualome fingerprint comprises a principle component analysis, a t-Distributed Stochastic Neighbor Embedding, a heat map, a diversity index, or a combination thereof.

62. A method for preparation of a menstrualome fingerprint, comprising:
(a) obtaining a first sample from a subject, wherein the first sample comprise cervicovaginal or menstrual fluid collected onto an absorbent sample collector;
(b) eluting the first sample from the sample collector into an aqueous buffer;
(c) separating a biological material from the first sample;
(d) constructing a sample menstrualome fingerprint, wherein the sample menstrualome fingerprint comprises the level and/or presence of a plurality of menstrualome biomarkers in the biological material from the first sample; and
(e) comparing the sample menstrualome fingerprint to a reference fingerprint.

63. The method of claim 62, wherein the reference fingerprint comprises the level and/or presence of a plurality of menstrualome biomarkers in a reference group of subjects.

64. The method of claim 62, wherein the reference fingerprint comprises the level and/or presence of a plurality of menstrualome biomarkers in the subject at a prior time point.

65. The method of claim 62, wherein reference menstrualome fingerprint comprises a threshold level or presence of the plurality of menstrualome biomarkers that are associated with a health state.

66. The method of claim 62, wherein the reference menstrualome fingerprint comprises a threshold level or presence of the plurality of menstrualome biomarkers that are associated with a health state.

67. The method of claim 66, wherein the plurality of menstrualome biomarkers comprise biomarkers that display differential presence or level in cervicovaginal or menstrual fluid between two or more health states.

68. The method of claim 67, wherein the two or more health states comprise before and after a medical treatment.

69. The method of claim 68, wherein the health state comprises a health state before surgery.

70. The method of claim 66, wherein the reference state comprises a health state after surgery.

71. The method of claim 67, wherein the health state comprises a menstrual disorder.

72. The method of claim 71, wherein the health state comprises endometriosis.

73. The method of claim 66, wherein the health state comprises a healthy patient.

74. The method of claim 62, wherein the biological material comprises one or more biological materials selected from the group consisting of a RNA, a DNA, a methylated nucleic acid, a miRNA, a protein, a protein-nucleic acid complex, a microorganism, and a mammalian cell type.

75. The method of claim 62, wherein constructing the sample menstrualome fingerprint in (d) comprises assaying the extracted biological material from the first sample and the second sample to identify a plurality of biomarkers.

76. The method of claim 62, wherein the plurality of menstrualome biomarkers comprise biomarkers that display differential presence or level in cervicovaginal or menstrual fluid as compared to peripheral blood, cervicovaginal tissue, or a longitudinal menstrual sample.

77. The method of claim 62, wherein the first sample and reference sample comprise biological material collected at a different time points from the subject.

78. The method of claim 62, wherein the sample collector is an intravaginal sample collector.

79. The method of claim 62, wherein the sample collector preserves a biological material in an intact state.

80. The method of claim 62, wherein the sample collector is capable of absorbing at least 3 ml of fluid.

81. The method of claim 62, wherein the sample collector is placed into a buffer subsequent to collecting the sample.

82. The method of claim 62, wherein the biological material is DNA and plurality of menstrualome biomarkers comprises methylation status of a plurality of loci.

83. The method of claim 62, wherein the biological material is RNA and plurality of menstrualome biomarkers comprises expression level of a plurality of genes.

84. The method of claim 62, wherein the biological material is RNA and plurality of menstrualome biomarkers comprises the presence and/or level of a plurality of miRNA.

85. The method of claim 62, wherein the biological material is cells and plurality of menstrualome biomarkers measures the presence and/or amount of one or more cell types.

86. The method of claim 62, wherein the biological material is DNA and plurality of menstrualome biomarkers measures the presence and/or level of one or more microorganisms.

87. The method of claim 62, wherein the biological material is DNA and plurality of menstrualome biomarkers measures the diversity of microorganisms.

88. The method of claim 62, wherein the health reference menstrualome fingerprint comprises a principle component analysis, a t-Distributed Stochastic Neighbor Embedding, a heat map, a diversity index, or a combination thereof.

89. A method for preparation of a menstrualome fingerprint, comprising:
(a) obtaining a first sample and a second sample from a subject having or suspected to have endometriosis, wherein the first sample and the second sample comprise cervicovaginal or menstrual fluid collected onto an absorbent sample collector;
(b) eluting the first sample and the second ample separately from the first and second sample collector into an aqueous buffer;
(c) separating a biological material from each of the first sample and the second sample; and
(d) constructing a sample menstrualome fingerprint, wherein the sample menstrualome fingerprint comprises the differential of the level and/or presence of a plurality of menstrualome biomarkers in the biological material from the first sample and the second sample.

90. The method of claim 89, wherein the biological material comprises one or more biological materials selected from the group consisting of a RNA, a DNA, a methylated nucleic acid, a miRNA, a protein, a protein-nucleic acid complex, a microorganism, and a mammalian cell type.

91. The method of claim 90, further comprising isolating the extracted biological material from the first sample and the second sample from other components of the first sample and the second sample.

92. The method of claim 90, wherein constructing the sample menstrualome fingerprint in (d) comprises assaying the extracted biological material from the first sample and the second sample to identify a plurality of biomarkers.

93. The method of claim 90, wherein the biological material is a miRNA and the plurality of biomarkers comprises a miRNA selected from the group consisting of let-7c-5p, miR-100-5p, miR-149-5p, miR-193b-3p, miR-221-5p, miR-363-3p, miR-99a-5p, let-7e-5p, miR-10a-5p, miR-10b-5p, miR-125b-5p, miR-127-3p, miR-132-3p, miR-141-3p, miR-142-5p, miR-143-3p, miR-144-5p, miR-145-5p, miR-152-3p, miR-16-2-3p, miR-17-3p, miR-195-5p, miR-196b-5p, miR-199a-3p/199b-3p, miR-200a-3p, miR-200c-3p, miR-203a-3p, miR-205-5p, miR-21-3p, miR-21-5p, miR-22-3p, miR-222-3p, miR-224-5p, miR-23b-3p, miR-27b-3p, miR-28-3p, miR-30a-3p, miR-30a-5p, miR-34a-5p, miR-34c-5p, miR-365a-3p/365b-3p, miR-375, miR-409, and miR-98-5p.

94. The method of claim 93, where the miRNA is selected from the group consisting of miR-1271-5p, miR-4485-3p, miR-125b-2-3p, and miR-410-3p.

95. The method of claim 90, wherein the plurality of biomarkers comprises a methylation profile of one or more CpG sites selected from the CpG sites in Table 4.

96. The method of claim 90, wherein the microorganism is a bacterium in a genus selected from the group consisting of Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, and Klebsiella.

97. The method of claim 90, wherein the mammalian cell type is selected from the group consisting of an endothelial cell, an epithelial cell, a leukocyte, a mesenchymal cell, and a combination thereof.

98. The method of claim 89, further comprising (e) comparing the sample menstrualome fingerprint to a reference menstrualome fingerprint.

99. The method of claim 98, wherein the reference menstrualome fingerprint comprises a threshold level or presence of the plurality of menstrualome biomarkers that are associated with a health state.

100. The method of claim 99, wherein the health state comprises a health state before surgery.

101. The method of claim 99, wherein the reference state comprises a health state after surgery.

102. The method of claim 89, wherein the first sample and second sample comprise biological material collected at a different time points from the subject.

103. The method of claim 89, wherein the time points are separated by a time period between about 15 minutes and about 30 days.

104. The method of claim 89, wherein the time points comprise different days within a menstrual cycle of the subject.

105. The method of claim 89, wherein the time points are within a single menstrual cycle.

106. The method of claim 89, wherein the time points comprise days in separate menstrual cycles.

107. The method of claim 89, wherein the time points are during one or more days of menstruation of the subject.

108. The method of claim 89, wherein one time point is during menstruation of the subject and one time point is not during menstruation of the subject.

109. The method of claim 89, wherein the sample collector is an intravaginal sample collector.

110. The method of claim 89, wherein the sample collector preserves a biological material in an intact state.

111. The method of claim 89, wherein the sample collector is capable of absorbing at least 3 ml of fluid.

112. The method of claim 89, wherein the sample collector is placed into a buffer subsequent to collecting the sample.
